(19) United States
(12) Patent Application Publication
(10) Pub. No.: US 2008/0194522 A1
(43) Pub. Date: Aug. 14, 2008

(54) Development of Fluorogenic Substrates For Monoamine Oxidases (Mao-A and Mao-B)

(76) Inventors: Gong Chen, New York, NY (US); Dominic J. Yee, La Jolla, CA (US); Niko Gubernator, New York, NY (US)

Correspondence Address: COOPER & DUNHAM, LLP, 1185 AVENUE OF THE AMERICAS, NEW YORK, NY, 10036, US

(21) Appl. No.: 11/661,152

(22) PCT Filed: Aug. 25, 2005
(86) PCT No.: PCT/US05/30276

§ 371 Date: Nov. 19, 2007

Related U.S. Application Data
(60) Provisional application No. 60/604,538, filed on Aug. 25, 2004.

(51) Int. Cl. A61K 31/404; C07D 491/06; C07D 407/10; C07F 5/02; A61K 31/436; A61P 25/00; A61K 31/69; C12Q 1/26; A61K 31/352

(52) U.S. Cl. 514/64; 549/403; 548/454; 548/405; 546/66; 435/25; 514/457; 514/414; 514/288

(57) ABSTRACT

The present invention relates to compounds useful for detecting the activity of monoamine oxidases, compounds useful for competitively inhibiting monoamine oxidases, for determining inhibitors of monoamine oxidases and compounds useful for treating monoamine oxidase-related nervous system pathologies, as well as pharmaceutical compositions and methods of manufacture thereof.

This application claims benefit of U.S. Provisional Application Ser. No. 60/604,538, filed Aug. 25, 2004, the contents of which are hereby incorporated by reference.

Throughout this application, various publications are referenced by complete citation in parentheses. The disclosures of these publications in their entireties are hereby incorporated by reference into this application in order to more fully describe the state of the art as known to those skilled therein as of the date of the invention described and claimed herein.

### BACKGROUND OF THE INVENTION

Molecular imaging of metabolic and signaling events in living systems represents an important frontier in life sciences and medicine. The ability to observe functioning cells, tissues, and organs with high levels of molecular and dynamic resolution will propel a wide spectrum of human activities, including scientific, philosophical, and medicinal fields (Weissleder, R. and Ntziachristos, V. Nature Med. 2003, 9, 123-128). One promising approach for non-invasive metabolic measurements stands on the use of small molecule reporters, such as fluorogenic probes which provide a measurable optical signal for a particular enzyme facilitated molecular process ((a) Moreira R., Havranek M., Sames D. J. Am. Chem. Soc. 2001, 123, 3927-3931. (b) Chen, C.-A.; Yeh, R.-H.; Lawrence, D. S. J. Am. Chem. Soc. 2002, 124, 3840-3841).

Noninvasive fluorescence imaging provides cellular study with high sensitivity and great versatility while minimally perturbing the cell under investigation (Weissleder, R.; Mahmood, U. Radiology 2001, 219:316-333). Imaging of metabolic and signaling events in live cells represents an important frontier in this area. By taking advantage of enzyme's promiscuity (or substrate flexibility), a synthetic molecule could function as a competitive substrate to its physiological substrate (Yarnell, A. In Chemical and Engineering News 2003, 81:33-35; and Handbook of Fluorescent Probes and Research Chemicals, Molecular Probes, 9th ed.; Haugland, R. P., Ed.; 2002, Molecular Probes: Eugene, Oreg.). Equipping the molecule with a “smart” built-in fluorescence switch mechanism could enable it report specific enzymatic activity with fluorescence signal (Yee, D. J.; Balsanek, V.; Sames, D. J. Am. Chem. Soc. 2004, 126:2282-283. (b) Chen, C. A.; Yeh, R. H.; Lawrence, D. S. J. Am. Chem. Soc. 2002, 124:3840-3841. (c) Badalassi, F.; Wahler, D.; Klein, G.; Crotti, P.; Reymond, J. L. Angew. Chem. Int. Ed. 2000, 39:4067-4070).

Monoamine oxidase (MAO) is an FAD-dependent enzyme and plays an essential role in the regulation of monoamine neurotransmitters such as dopamine and serotonin (Castagnoli, N.; Dalvie, D.; Kalgutkar, A.; Taylor, T. Chem. Res. Toxicol. 2001, 14:1139-1162). It catalyzes the anaerobic conversion of amine substrates to the corresponding imines, which are released from the enzyme and hydrolyzed to the corresponding aldehydes (FIG. 1A) (Silverman, R. B. Acc. Chem. Res. 1995, 28:335-342). MAO is found to be a relatively promiscuous enzyme and can catalyze the oxidation of a variety of exogenous amines with a special preference for primary amines. Because of its important physiological functions, which have been widely implicated in apoptosis, immunosuppression, cytotoxicity, cell growth, and proliferation, MAO has been a crucial target of some pharmaceutical research particularly on neurological diseases. Effective imaging of its in vivo activity will provide a fundamentally new method in biological and medicinal application (Zhou, J. J. P.; Zhong, B.; Silverman, R. B. Anal. Biochem. 1996, 234:9; Nicotra, A.; Parvez, S. H. Biogenic Amines 1999, 15:307-320).

Here, in the context of monoamine oxidases, design, chemical synthesis, enzymatic screening, identification of leads, and development of new fluorogenic probes, non-physiological substrates and competitive inhibitors for MAOs are disclosed.

### SUMMARY OF THE INVENTION

One embodiment of this invention provides a compound of having the structure:

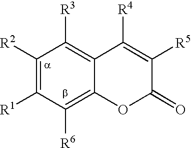

wherein R¹ is —H, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical,
or R¹ and R² form an unsubstituted pyrrole,
or R¹ and R⁶ form an unsubstituted pyrrole,
or R¹ forms an octahydro-quinolizine with R² and R⁶,
wherein each of R², R³, R⁴, R⁵, or R⁶ is independently —H, —OH, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, halide, indole radical, or R² and R³ form a pyrrole,
wherein when R¹ and R² form a pyrrole, a N of the pyrrole is covalently bound to carbon α,
wherein when R¹ and R⁶ form a pyrrole, then R⁴ is —H,
wherein when R¹ is —N(alkyl)₂ or —NH₂, then R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, CX₃ where X is a halide, or indole radical,
wherein when R¹ is H, R³ is —NH₂ and R² is H; or R² and R³ form a pyrrole; or R² is —NH₂ then R⁴ is alkyl,
wherein when R¹ is CH₃, R² is —H, —OH, alkyl, alkenyl, alkynyl, substituted or unsubstituted aryl, cycloalkyl, substituted or unsubstituted heteroaryl, —NH-alkyl, —N(alkyl)₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical, and R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,
or a salt or stereoisomer thereof.

### BRIEF DESCRIPTION OF THE FIGURES

FIG. 1A-1B. 1A: Mechanism of oxidative deamination of the substrate by MAO. 1B: Design of fluorescence switch probe based on a cascade reporting pathway.

FIG. 2A-2D. 2A and 2B: Design and fluorescence spectra of generation I probes based on PET quenching A is broken line, B is solid line); 2C, 2D: Design and fluorescence spectra of generation II probes based on TICT quenching (e.g. 5 μM, pH 7, buffer) (C is broken line, D is solid).

FIG. 3. Different aminocoumarins (5,6,7,8-amino) and their corresponding pyrrolocoumarins.

FIG. 4A-4B. 4A: fluorescence spectra of enzymatic assays; 4B: Kinetic parameters for probe 9 and physiological substrate for MAO-B.

FIG. 5. Fluorescence Spectra of Probes 3 (dashed) and 4 (solid).

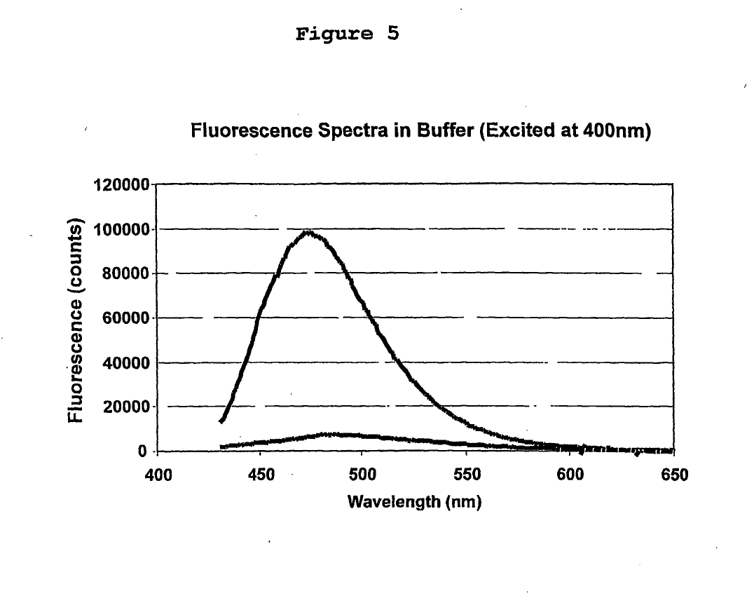

FIG. 6. Fluorescence Spectra of Probes 16 (dashed) and 17 (solid).

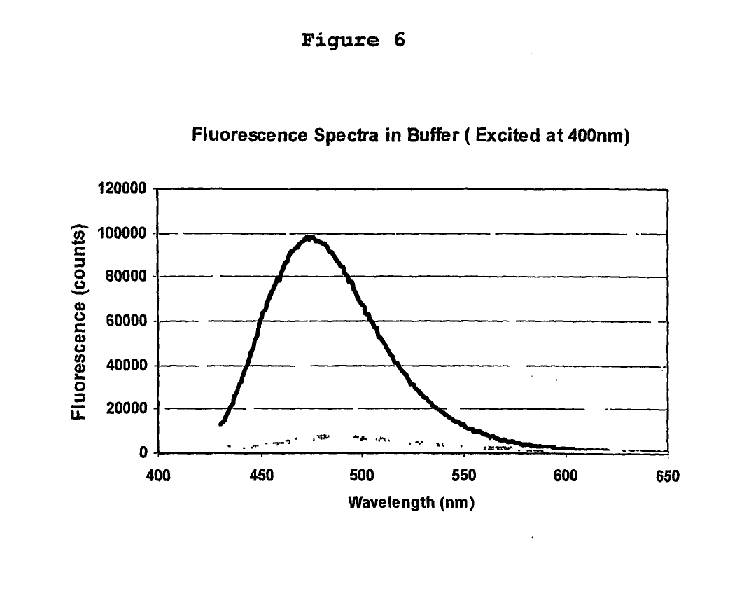

FIG. 7. Fluorescence Spectra of Probes 23 (dashed) and 24 (solid).

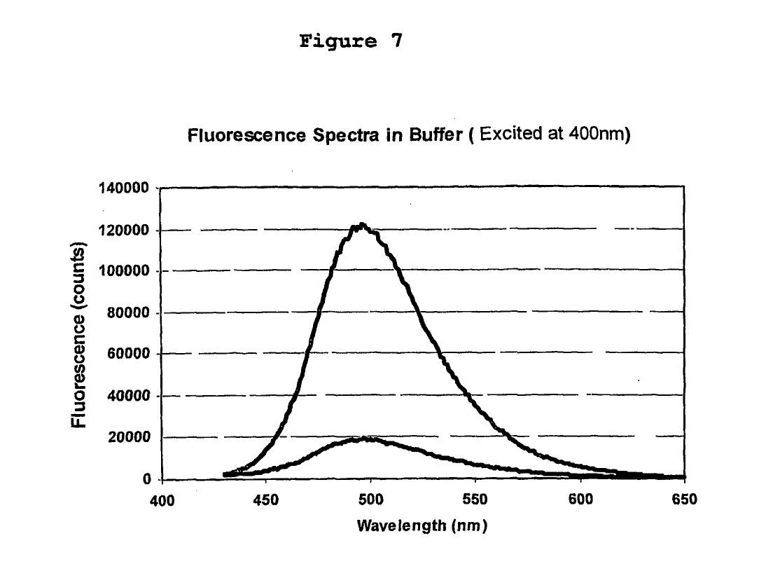

FIG. 8. Fluorescence spectra for indole probes, compounds 114, 126 and 128.

FIG. 9. Fluorescence Spectra of Probes 17 (dashed) and 28 (solid).

FIG. 10. Fluorescence emission spectra of probe 36 (dashed) and product (solid).

FIG. 11. Fluorescence Spectra of Probes 52 and 57.

FIG. 12. Fluorescence Spectra of Probes 58 and 59.

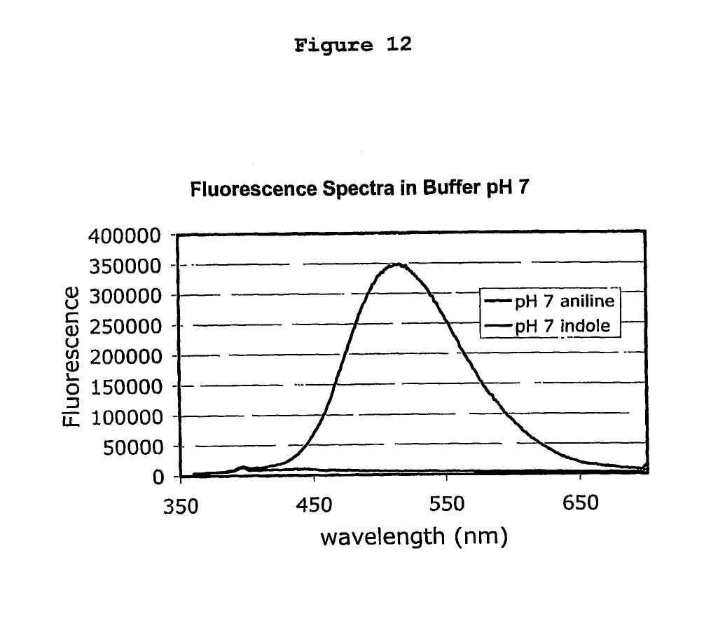

FIG. 13. The spectrum of indole (XX) dissolved in different organic solvents.

FIG. 14. pH dependence spectrum of indole (XX).

FIG. 15. Diamine III conversion to indole XX spectras.

FIG. 16. Fluorescence growth curve of product III.

### DETAILED DESCRIPTION

Some abbreviations used herein:

CH₂Cl₂: dichloromethane

ClCH₂CH₂Cl: 1,2-dichloroethane

DBS—NH₂: dibenzosuberylamine

DMF: dimethylformamide

EtOAc: ethyl acetate

NaBH(OAc)₃: sodium triacetoxyborobydride

PtO₂: platinum (IV) oxide

SnCl₂.2H₂O: tin (II) chloride dihydrate

TES: triethylsilane

TFA: trifluroacetic acid

As used herein, “MAO” means monoamine oxidase.

As used herein, “reference standard” means a normalized value obtained form a normal sample, and in the case of fluorescence means the normalized fluorescence measured form a non-cancerous or other standardized sample as measured by a parallel assay with the same steps and conditions to which the tested or cancerous sample is being subjected.

As used herein, a “competitive inhibitor” in relation to an enzyme is a substance capable of binding to the enzyme's active site so preventing the enzyme from binding its substrate.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. For example, an amount effective to inhibit or reverse depressive disorder or anxiety disorder symptoms, or for example to inhibit, attenuate or reverse neurodegenerative disorder symptoms. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, “treatment” of a depressive, anxiety or neurodegenerative disorder encompasses inducing inhibition, regression, or stasis/prevention of the disorder. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “salt” is salt of the instant compounds which has been modified by making acid or base salts of the compounds. In the case of compounds used for treatment of cancer, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutical carrier.

As used herein “medium” shall include any physiological medium or artificial medium of that supports monoamine oxidase activity, whether the MAO is cellular or is contained within a lysate or in a purified form. Preferably, the fluorescence of the medium should be negligible or constant.

As used herein, a “reduction” when pertaining to fluorescence can mean either a reduction in the relative or absolute amount of fluorescence, or a reduction in the rate of change of fluorescence, whether the rate of change be positive or negative.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds may comprise a single compound or mixtures thereof with other anti-cancer compounds, other cancer or tumor growth inhibiting compounds. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection or other methods, into the cancer, all using dosage forms well known to those of ordinary skill in the pharmaceutical arts.

The compounds can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Specific examples of pharmaceutical acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues.

Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The active ingredient can be administered orally in solid dosage forms, such as capsules, tablets, and powders, or in liquid dosage forms, such as elixirs, syrups, and suspensions. It can also be administered parentally, in sterile liquid dosage forms.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The instant compounds may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The present invention also includes pharmaceutical kits useful, for example, for the treatment of depressive, anxiety or neurodegenerative disorders, which comprise one or more containers containing a pharmaceutical composition comprising an effective amount of one or more of the compounds. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 . . . , n-1 or n carbons in a linear or branched arrangement. For example, C₁-C₆, as in “C₁-C₆alkyl” is defined to include groups having 1, 2, 3, 4, 5, or 6 carbons in a linear or branched arrangement, and specifically includes methyl, ethyl, propyl, butyl, pentyl, hexyl, and so on. With regard to “alkyl”, R¹ through R⁶ as used here are C₁-C₆. “Alkoxy” represents an alkyl group of indicated number of carbon atoms attached through an oxygen bridge.

The term “alkyl” as used in the terms “-alkyl-OH”, “—NH-alkyl”, “-alkyl-(NH₂)”, “-alkyl-C(O)(OH”, and “—O-alkyl” are C₁-C₆ alkyl as defined above, i.e. they include groups having 1, 2, 3, 4, 5, or 6 carbons in a linear or branched arrangement. For example methyl, ethyl, propyl, butyl, pentyl, or hexyl in a linear or branched arrangement.

The term “alkyl” as used in the term “—N(alkyl)₂” means C₁-C₆ alkyl as defined above, i.e. they include groups having 1, 2, 3, 4, 5, or 6 carbons in a linear or branched arrangement. However, the two alkyl groups of “—N(alkyl)₂” need not necessarily be the same type of alkyl group. For example one alkyl may be chosen from the group methyl, ethyl, propyl, butyl, pentyl, or hexyl in a linear or branched arrangement and the other alkyl may be independently chosen from the group methyl, ethyl, propyl, butyl, pentyl, or hexyl.

The term “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

If no number of carbon atoms is specified, the term “alkenyl” refers to a non-aromatic hydrocarbon radical, straight or branched, containing at least 1 carbon to carbon double bond, and up to the maximum possible number of non-aromatic carbon-carbon double bonds may be present. For example, “C₂-C₆ alkenyl” includes an alkenyl radical having 2, 3, 4, 5, or 6 carbon atoms, and 1, 2, 3, 4, or 5 carbon-carbon double bonds as appropriate. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. With regard to “alkenyl”, R¹ through R⁶ as used here are C₂-C₆

The term “cycloalkenyl” shall mean cyclic rings of 3 to 10 carbon atoms and at least 1 carbon to carbon double bond (i.e., cycloprenpyl, cyclobutenyl, cyclopenentyl, cyclohexenyl, cycloheptenyl or cycloocentyl).

The term “alkynyl” refers to a hydrocarbon radical straight or branched, containing at least 1 carbon to carbon triple bond, and up to the maximum possible number of non-aromatic carbon-carbon triple bonds may be present. Thus, “C₂-C₆ alkynyl” includes an alkynyl radical radical having 2 to 3 carbon atoms and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. With regard to “alkynyl”, R² through R⁶ as used here are C₂-C₆.

As used herein, “aryl” is intended to mean any stable. monocyclic, bicyclic or tricyclic carbon ring of up to 10 atoms in each ring, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring. The substituted aryls included in this invention include substitution at any suitable position with amines, substituted amines, alkylamines, hydroxys and alkylhydroxys, wherein the “alkyl” portion of the alkylamines and alkylhydroxys is a C₂-C₆ alkyl as defined hereinabove. The substituted amines may be substituted with alkyl, alkenyl, alkynl, or aryl groups as hereinabove defined.

The term “heteroaryl”, as used herein, represents a stable monocyclic or bicyclic ring of up to 10 atoms in each ring, wherein at least one ring is aromatic and contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

As appreciated by those of skill in the art, “halo”, “halide”, or “halogen” as used herein is intended to include chloro, fluoro, bromo and iodo.

The term “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

The alkyl, alkenyl, alkynyl, cycloalkyl, aryl, heteroaryl and heterocyclyl substituents may be unsubstituted or unsubstituted, unless specifically defined otherwise. For example, a (C₁-C₆) alkyl may be substituted with one or more substituents selected from OH, oxo, halogen, alkoxy, dialkylamino, or heterocyclyl, such as morpholinyl, piperidinyl, and so on.

In the compounds of the present invention, alkyl, alkenyl, alkynyl, cycloalkyl, cycloalkenyl, heterocyclyl and heteroaryl groups can be further substituted by replacing one or more hydrogen atoms be alternative non-hydrogen groups. These include, but are not limited to, halo, hydroxy, mercapto, amino, carboxy, cyano and carbamoyl.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing compounds of the present invention, one of ordinary skill in the art will recognize that the various substituents, i.e. R¹ through R⁶ are to be chosen in conformity with well-known principles of chemical structure connectivity.

The compounds of the present invention are available in racemic form or as individual enantiomers. For convenience, some structures are graphically represented as a single enantiomer but, unless otherwise indicated, is meant to include both racemic and enantiomerically pure forms. Where cis and trans sterochemistry is indicated for a compound of the present invention, it should be noted that the stereochemistry should be construed as relative, unless indicated otherwise. For example, a (+) or (−) designation should be construed to represent the indicated compound with the absolute stereochemistry as shown.

Racemic mixtures can be separated into their individual enantiomers by any of a number of conventional methods. These include, but are not limited to, chiral chromatography, derivatization with a chiral auxillary followed by separation by chromatography or crystallization, and fractional crystallization of diastereomeric salts. Deracemization procedures may also be employed, such as enantiomeric protonation of a pro-chiral intermediate anion, and the like.

The methods of the present invention when pertaining to cells, and samples derived or purified therefrom, including enzyme containing fractions, may be performed in vitro. The methods of treatment may, in different embodiments, be performed in vivo, in situ, or in vitro. The methods of diagnosis may, in different embodiments, be performed in vivo, in situ, or in vitro.

This invention provides a compound having the structure:

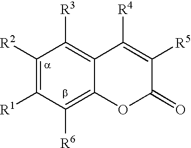

wherein R¹ is —H, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical,

or R¹ and R² form an unsubstituted pyrrole,

or R¹ and R⁶ form an unsubstituted pyrrole

or R¹ forms an octahydro-quinolizine with R² and R⁶,

wherein each of R², R³, R⁴, R⁵, or R⁶ is independently —H, —OH, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, halide, indole radical, or R² and R³ form a pyrrole,

wherein when R¹ and R² form a pyrrole, a N of the pyrrole is covalently bound to carbon α,

wherein when R¹ and R⁶ form a pyrrole, then R⁴ is —H,

wherein when R¹ is —N(alkyl)₂ or —NH₂, then R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,

wherein when R¹ is H, R³ is —NH₂ and R² is H; or R² and R³ form a pyrrole; or R² is —NH₂ then R⁴ is alkyl,

wherein when R¹ is CH₃, R² is —H, —OH, alkyl, alkenyl, alkynyl, substituted or unsubstituted aryl, cycloalkyl, substituted or unsubstituted heteroaryl, —NH-alkyl, —N(alkyl)₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical, and R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,

or a salt or stereoisomer thereof.

In one embodiment of the invention, the alkyl is a C₁ to C₈ alkyl. In a further embodiment it is a C₁-C₆ alkyl.

In one embodiment of the invention when R² and R³ form a pyrrole, R¹ is alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical or —OH, alkyl, alkenyl, alkynyl, substituted or R⁶ is unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, halide, indole radical.

This invention further provides the instant compound, wherein the indole radical has the structure:

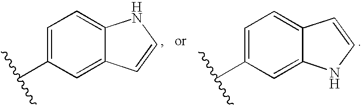

This invention further provides the instant compound, wherein the substituted aryl has the structure:

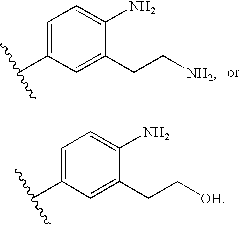

This invention further provides the instant compound, having the structure:

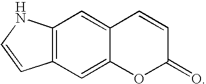

This invention further provides the instant compound, having the structure:

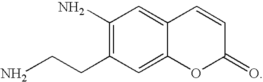

This invention further provides the instant compound, having the structure:

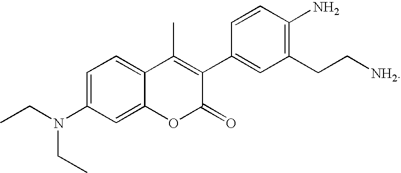

This invention further provides the instant compound, having the structure:

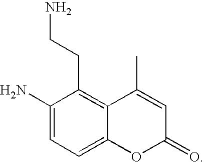

This invention further provides the instant compound, having the structure:

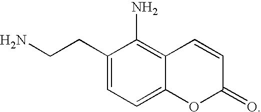

This invention further provides the instant compound, having the structure:

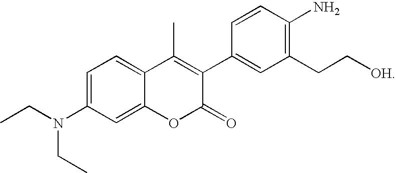

This invention further provides the instant compound, having the structure:

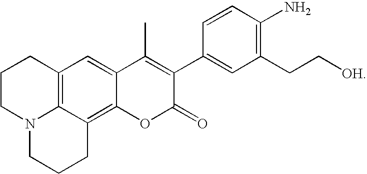

This invention further provides the instant compound, having the structure:

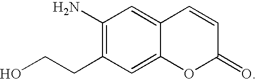

This invention further provides the instant compound, having the structure:

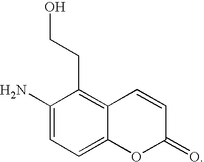

This invention further provides the instant compound, having the structure:

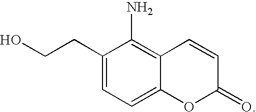

This invention further provides the instant compound, having the structure:

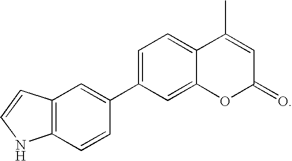

This invention further provides the instant compound, having the structure:

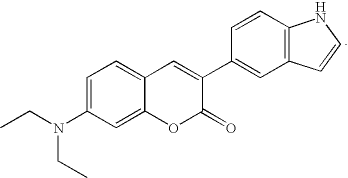

This invention further provides the instant compound, having the structure:

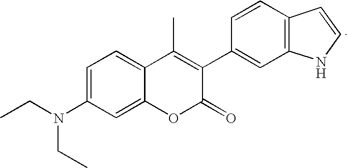

This invention further provides the instant compound, having the structure:

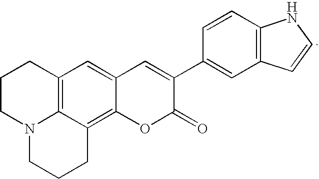

This invention further provides the instant compound, having the structure:

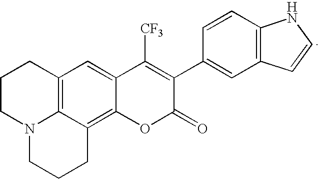

This invention further provides the instant compound, having the structure:

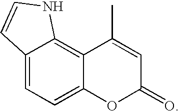

This invention further provides the instant compound, having the structure:

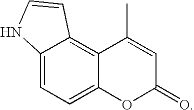

This invention further provides the instant compound, having the structure:

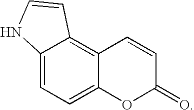

In the embodiments of this invention a first suitable solvent, second suitable solvent, third suitable solvent and so forth may be different from one another, or such solvents may the same, or any combination thereof.

This invention also provides a process of obtaining a compound which is an inhibitor of a monoamine oxidase comprising:
a) providing the monoamine oxidase in a medium;
b) contacting the monoamine oxidase with a known compound that undergoes a detectable increase in fluorescence when oxidized in the presence of the monoamine oxidase under conditions permitting oxidative deamination of the known compound in the presence of the monoamine oxidase;
c) detecting an increase in the fluorescence emitted by the known compound;
d) contacting the monoamine oxidase with a compound to be tested for activity as an inhibitor of monoamine oxidase;
e) detecting a change in the fluorescence of the known compound; and
f) recovering the test compound,
wherein a decrease in the fluorescence emitted by the known compound detected in step e) compared to step c) indicates that the test compound is an inhibitor of monoamine oxidase.

This invention further provides the instant process, wherein the monoamine oxidase is MAO-B.

This invention further provides the instant process, wherein the monoamine oxidase is MAO-A.

This invention further provides the instant process, wherein the known compound has the structure:

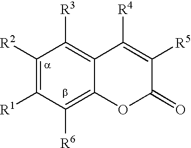

wherein R¹ is —H, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical,

or R¹ and R² form an unsubstituted pyrrole,

or R¹ and R⁶ form an unsubstituted pyrrole

or R¹ forms an octahydro-quinolizine with R² and R⁶,

wherein each of R², R³, R⁴, R⁵, or R⁶ is independently —H, —OH, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, halide, indole radical, or R² and R³form a pyrrole,

wherein when R¹ and R² form a pyrrole, a N of the pyrrole is covalently bound to carbon α,

wherein when R¹ and R⁶ form a pyrrole, then R⁴ is —H,

wherein when R¹ is —N(alkyl)₂ or —NH₂, then R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,

wherein when R¹ is H, R³ is —NH₂ and R² is H; or R² and R³ form a pyrrole; or R² is —NH₂ then R⁴ is alkyl,

wherein when R¹ is CH₃, R² is —H, —OH, alkyl, alkenyl, alkynyl, substituted or unsubstituted aryl, cycloalkyl, substituted or unsubstituted heteroaryl, —NH-alkyl, —N(alkyl)₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical, and R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,

or a salt or stereoisomer thereof.

This invention further provides the instant process, wherein the known compound has the structure set forth below:

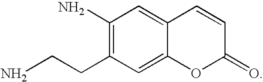

This invention further provides the instant process, wherein the known compound has one of the structures set forth below:

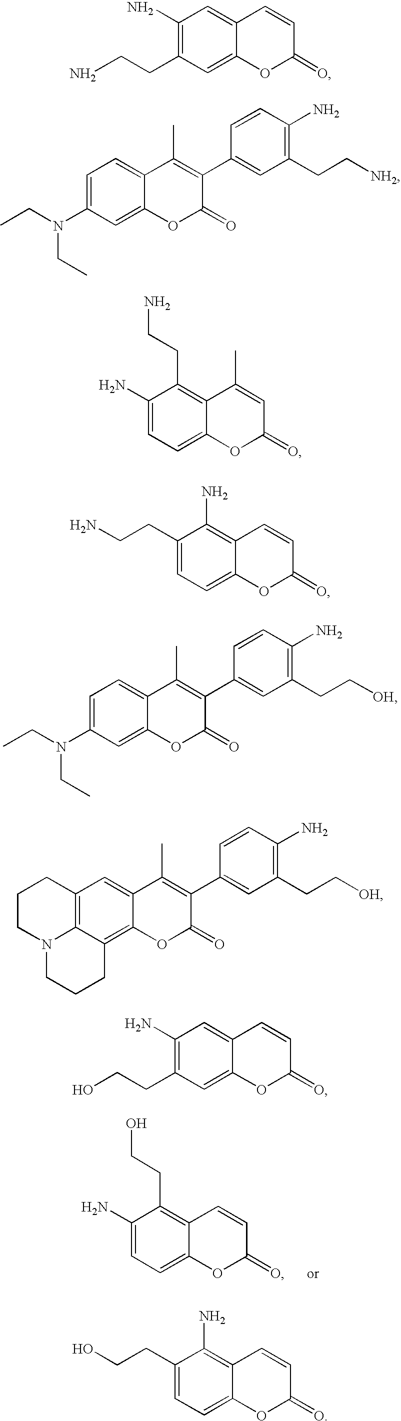

MAO activity can result in production of hydrogen peroxide. H₂O₂ production can be measured chemically or fluorescently. An example of the latter method is employing the commercially available AMPLEX Red Monoamine Oxidase detection kit from Molecular Probes, Eugene, Oreg. It is notable that for a drug candidate that is an amine, it is desirable to establish whether the drug candidate is (1) an inhibitor of MAO or (2) substrate of MAO. The probes disclosed here allow for a continuous MAO assay to determine this, which can be easily done in a high throughput format (e.g. plate reader). This method can be performed in conjunction with measurement of H₂O₂ production if desired.

This invention also provides a method of treating a depressive disorder, an anxiety disorder, or a neurodegenerative disease in a subject comprising administering to the subject an effective amount of a compound having the structure:

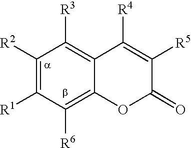

wherein R¹ is —H, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical,

or R¹ and R² form an unsubstituted pyrrole,

or R¹ and R⁶ form an unsubstituted pyrrole

or R¹ forms an octahydro-quinolizine with R² and R⁶,

wherein each of R², R³, R⁴, R⁵, or R⁶ is independently —H, —OH, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, halide, indole radical, or R² and R³ form a pyrrole,

wherein when R¹ and R² form a pyrrole, a N of the pyrrole is covalently bound to carbon α,

wherein when R¹ and R⁶ form a pyrrole, then R⁴ is —H,

wherein when R¹ is —N(alkyl)₂ or —NH₂, then R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,

wherein when R¹ is H, R³ is —NH₂ and R² is H; or R² and R³ form a pyrrole; or R² is —NH₂ then R⁴ is alkyl,

wherein when R¹ is CH₃, R² is —H, —OH, alkyl, alkenyl, alkynyl, substituted or unsubstituted aryl, cycloalkyl, substituted or unsubstituted heteroaryl, —NH-alkyl, —N(alkyl)₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical, and R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,

or a salt or stereoisomer thereof.

This invention further provides the instant method, wherein the compound has the structure:

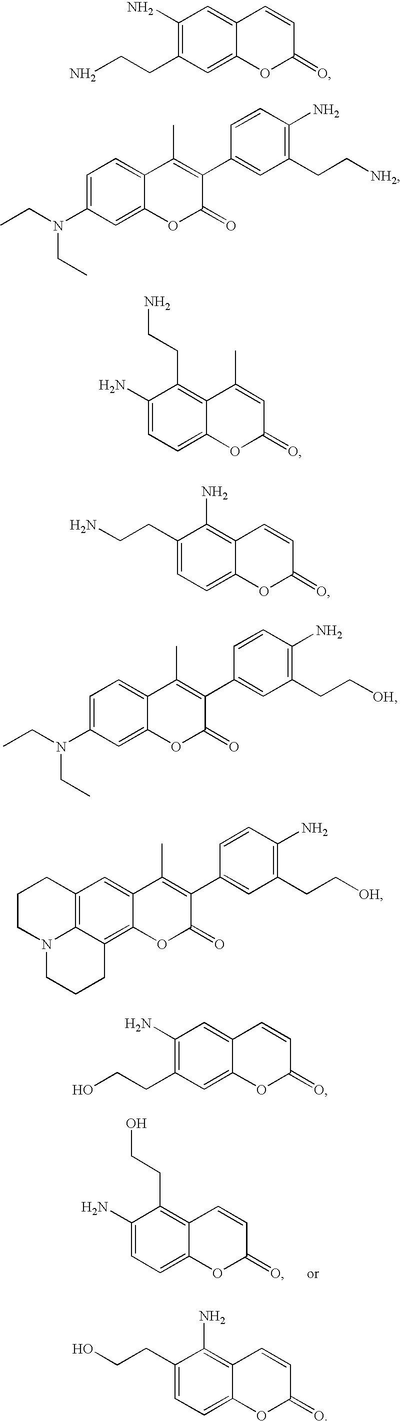

This invention also provides a method for detecting an active monoamine oxidase in a nervous system tissue comprising:
a) providing a sample of the nervous system tissue;
b) contacting the sample with a known compound which undergoes a detectable increase in fluorescence when oxidized in the presence of a monoamine oxidase under conditions permitting oxidative deamination of the compound in the presence of the monoamine oxidase;
c) detecting an increase in the fluorescence of the known compound;

wherein an increase in the fluorescence of the sample detected in step c) indicates the presence of an active monoamine oxidase in the nervous system tissue.

This invention further provides the instant method, wherein the known compound has the structure:

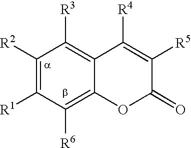

wherein R¹ is —H, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical,

or R¹ and R² form an unsubstituted pyrrole,

or R¹ and R⁶ form an unsubstituted pyrrole,

or R¹ forms an octahydro-quinolizine with R² and R⁶,

wherein each of R², R³, R⁴, R⁵, or R⁶ is independently —H, —OH, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, halide, indole radical, or R² and R³ form a pyrrole,

wherein when R¹ and R² form a pyrrole, a N of the pyrrole is covalently bound to carbon α,

wherein when R¹ and R⁶ form a pyrrole, then R⁴ is —H,

wherein when R¹ is —N(alkyl)₂ or —NH₂, then R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,

wherein when R¹ is H, R³ is —NH₂ and R² is H; or R² and R³ form a pyrrole; or R²is —NH₂ then R⁴ is alkyl,

wherein when R¹ is CH₃, R² is —H, —OH, alkyl, alkenyl, alkynyl, substituted or unsubstituted aryl, cycloalkyl, substituted or unsubstituted heteroaryl, —NH-alkyl, —N(alkyl)₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical, and R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,

or a salt or stereoisomer thereof.

This invention further provides the instant method, wherein the compound has the structure:

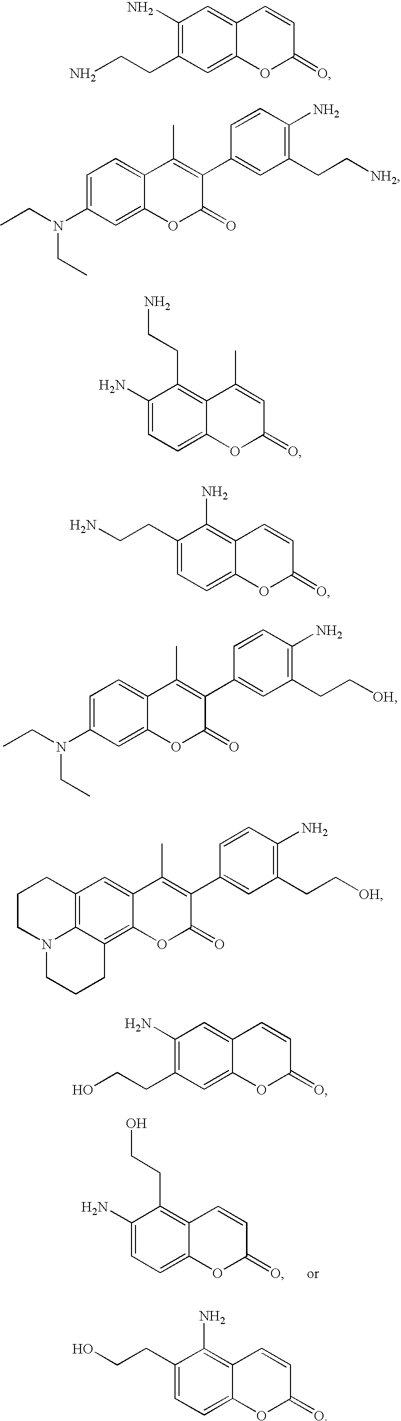

This invention also provides a composition comprising a pharmaceutically acceptable carrier and a compound having the structure:

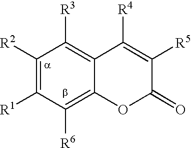

wherein R¹ is —H, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical,

or R¹ and R² form an unsubstituted pyrrole,

or R¹ and R⁶ form an unsubstituted pyrrole

or R¹ forms an octahydro-quinolizine with R² and R⁶,

wherein each of R², R³, R⁴, R⁵, or R⁶is independently —H, —OH, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, halide, indole radical, or R² and R³ form a pyrrole,

wherein when R¹ and R² form a pyrrole, a N of the pyrrole is covalently bound to carbon α,

wherein when R¹ and R⁶ form a pyrrole, then R⁴ is —H,

wherein when R¹ is —N(alkyl)₂ or —NH₂, then R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,

wherein when R¹ is H, R³ is —NH₂ and R² is H; or R² and R³ form a pyrrole; or R² is —NH₂ then R⁴ is alkyl,

wherein when R¹ is CH₃, R² is —H, —OH, alkyl, alkenyl, alkynyl, substituted or unsubstituted aryl, cycloalkyl, substituted or unsubstituted heteroaryl, —NH-alkyl, —N(alkyl)₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical, and R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,

or a salt or stereoisomer thereof.

This invention further provides the instant composition, wherein the compound has the structure:

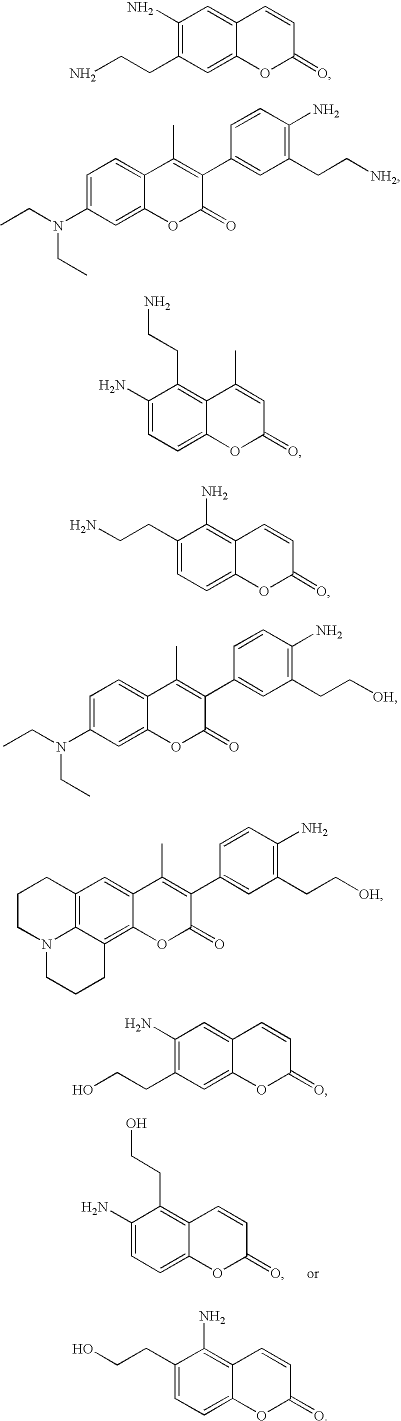

This invention also provides a process for making a composition comprising admixing a carrier and an amount of a compound having the structure:

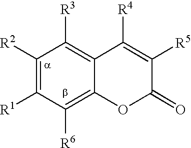

wherein R¹ is —H, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical,

or R¹ and R² form an unsubstituted pyrrole,

or R¹ and R⁶ form an unsubstituted pyrrole

or R¹ forms an octahydro-quinolizine with R² and R⁶,

wherein each of R², R³, R⁴, R⁵, or R¹ is independently —H, —OH, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, halide, indole radical, or R² and R³ form a pyrrole,

wherein when R¹ and R² form a pyrrole, a N of the pyrrole is covalently bound to carbon α,

wherein when R¹ and R⁶ form a pyrrole, then R⁴ is —H,

wherein when R¹ is —N(alkyl)₂ or —NH₂, then R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,

wherein when R¹ is H, R³ is —NH₂ and R² is H; or R² and R³ form a pyrrole; or R² is —NH₂ then R⁴ is alkyl,

wherein when R¹ is CH₃, R² is —H, —OH, alkyl, alkenyl, alkynyl, substituted or unsubstituted aryl, cycloalkyl, substituted or unsubstituted heteroaryl, —NH-alkyl, —N(alkyl)₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical, and R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,

or a salt or stereoisomer thereof.

This invention further provides the instant process, wherein the compound has the structure:

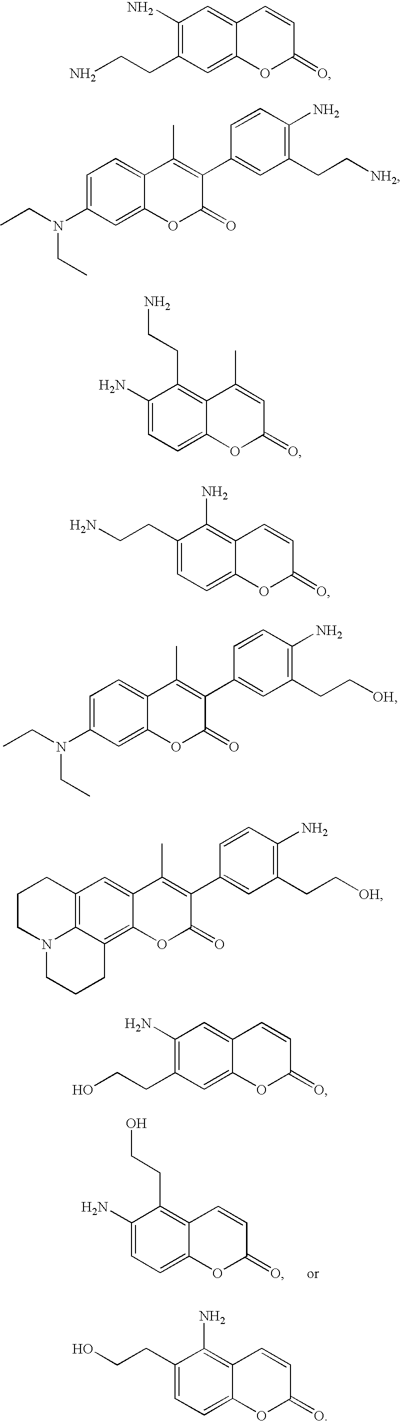

This invention further provides the instant process, wherein the compound is present in the composition in an amount effective to treat a depressive disorder, an anxiety disorder, or a neurodegenerative disorder.

This invention further provides a compound having any of the structures disclosed herein, as well as a process of producing such a compound as disclosed herein. All combinations of the various elements are within the scope of the invention.

This invention provides process for producing the instant compounds comprising:
contacting a compound having the structure:

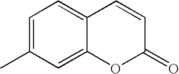

with a suitable nitrating agent in a suitable acid so as to produce a compound having the structure:

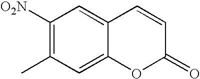

b) condensing the product of step a) in a first suitable solvent in the presence of Me₂NCH(OMe)₂ so as to produce a compound having the structure:

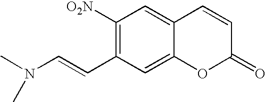

c) hydrolyzing the product of step b) in a refluxing second suitable solvent and aqueous HCL so as to produce a compound having the structure:

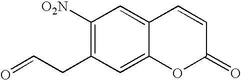

d) hydrogenating the product of step c) with a suitable catalyst in a third suitable solvent so as to produce the instant compound.

This invention provides process for producing the instant compounds comprising:
a) contacting a compound having the structure:

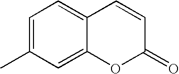

with a suitable nitrating agent in a suitable acid so as to produce a compound having the structure:

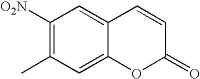

b) condensing the product of step a) in a first suitable solvent in the presence of Me₂NCH(OMe)₂ SO as to produce a compound having the structure:

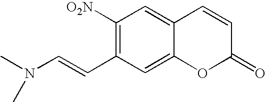

c) hydrolyzing the product of step b) in a refluxing second suitable solvent and aqueous HCL so as to produce a compound having the structure:

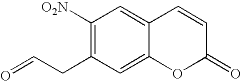

d) reducing the aldehyde of the product of step c) using a suitable reducing agent in a fourth suitable solvent so as to produce a compound having the structure:

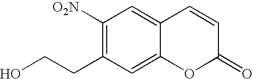

e) hydrogenating the product of step d) with a suitable catalyst in a third suitable solvent so as to produce the instant compound.

This invention provides the instant processes wherein the suitable catalyst is PtO₂, the suitable acid is H₂SO₄, the first suitable solvent is DMF, the second suitable solvent is ether, the third suitable solvent is EtOH or EtOAc or EtOH/EtOAc, the fourth suitable solvent is EtOH/CH₂Cl₂, and/or the suitable reducing agent is NaBH₄.

This invention provides process for producing the instant compounds comprising:
reductive amination of a compound having the structure:

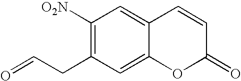

with a suitable reducing agent and a suitable animating agent in a first suitable solvent so as to produce a compound having the structure:

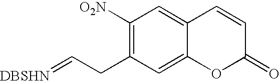

reducing the amino group of the product of step a) by exposing the product of step a) to a second suitable reducing agent in a refluxing second suitable solvent so as to produce a compound having the structure:

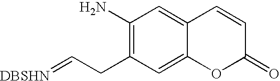

removal of the amino-protecting DBS group of the product of step b) by exposing the product of step b) to refluxing TFA and quenching with triethylsilane so as to produce the instant compound.

This invention provides the instant processes wherein the first suitable solvent is ClCH₂CH₂Cl₂, the suitable reducing agent is NaBH(OAc)₃, the suitable animating agent is DBS—NH₂, wherein the second suitable reducing agent is SnCl₂.2H₂O, and/or the second suitable solvent is EtOH.

This invention provides process for producing the instant compounds comprising:
a) exposing a product having the structure:

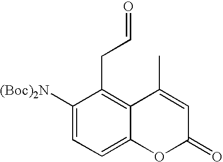

to a suitable reducing agent and a suitable animating agent in a first suitable solvent so as to produce a compound having the structure:

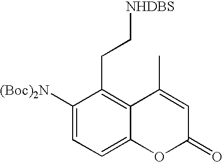

removing of the amino-protecting DBS group of the product of step a) by exposing the product of step b) to refluxing TFA and quenching with triethylsilane so as to produce the instant compound.

This invention provides the instant process, wherein the first suitable solvent is ClCH₂CH₂Cl₂, the suitable reducing agent is NaBH(OAc)₃, and/or the suitable animating agent is DBS—NH₂.

This invention provides process for producing the instant compounds comprising:
a) exposing a compound having the structure:

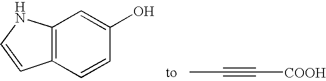

in the presence of DCC and DMAP in a suitable solvent so as to produce a compound having the structure:

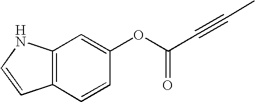

b) exposing the product of step a) to a suitable catalyst and a suitable solvent so as to produce the instant compound.

This invention provides process for producing the instant compounds comprising:
a) exposing a compound having the structure:

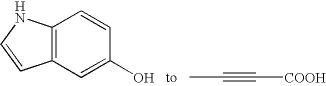

in the presence of DCC and DMAP in a first suitable solvent,
or to ethyl propynoate in the presence of Pd(OAc)₂ in TFA/CH₂Cl₂ or in the presence of Pd₂(dba)₃ in HCOOH,
or to a compound having the structure

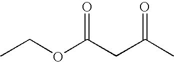

in the presence of concentrated H₂SO₄, so as to produce a compound having the structure:

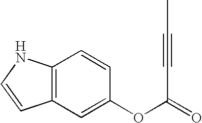

b) exposing the product of step a) to a suitable catalyst and a second suitable solvent so as to produce the instant compound.

This invention provides the instant process wherein the suitable catalyst is PtCl₄, wherein the first suitable solvent is 1,2-dichloroethane/DMF, and/or wherein the second suitable solvent is dioxane/1,2-dichloroethane.

This invention provides a process for producing the instant compound comprising:
a) exposing a compound having the structure:

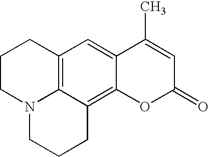

to a brominating agent in a first suitable solvent so as to produce a compound having the structure:

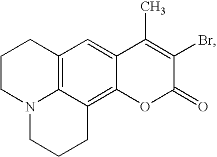

b) coupling the product the product of step a) to a compound having the structure:

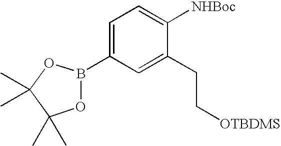

in the presence of a suitable catalyst and a suitable base in dioxane so as to produce a compound having the structure:

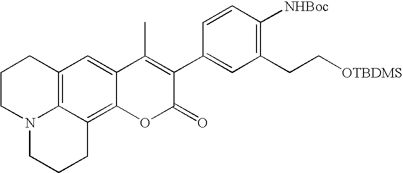

c) deprotecting the product of step b) with TBAF in THF and then TFA in CH₂Cl₂ so as to produce the instant compound

This invention provides the instant process wherein the suitable base is K₃PO₄, the suitable catalyst is PdCl₂(dppf), the first suitable solvent is CH₃CN, and/or the brominating agent is NBS.

This invention provides a process for producing the instant compound comprising:
a) exposing a compound having the structure:

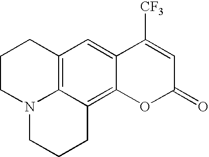

to a brominating agent in a first suitable solvent so as to produce a compound having the structure:

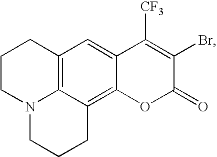

b) coupling the product the product of step a) to a compound having the structure:

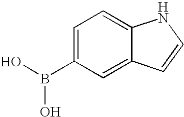

in the presence of a suitable catalyst and a suitable base in a second suitable solvent so as to produce the instant compound.

This invention provides the instant process wherein, wherein the suitable base is K₂CO₃, the suitable catalyst is PdCl₂(dppf), the first suitable solvent is CH₃CN, the second suitable solvent is DMF/H₂O, and/or the brominating agent is NBS.

This invention provides a process for producing the instant compound comprising:
a) brominating a compound having the structure:

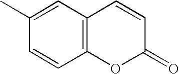

with a suitable brominating agent in the presence of benzoylperoxide in a first suitable solvent so as to produce a compound having the structure:

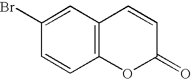

exposing the product of step a) to KCN in a second suitable solvent so as to produce a compound having the structure:

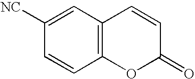

exposing the product of step b) to AcOH and H₂SO₄ in water so as to produce a compound having the structure:

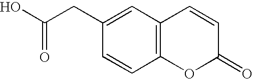

reducing the product of step c) with a suitable reducing agent in a third suitable solvent so as to produce a compound having the structure:

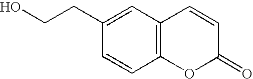

nitrating the product of step c) with a suitable nitrating agent and a suitable acid in a fourth suitable solvent so as to produce a compound having the structure:

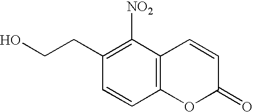

catalytically hydrogenating the nitro group of the product of step e) with H₂ and a suitable catalyst in a fifth suitable solvent so as to produce a compound having the structure:

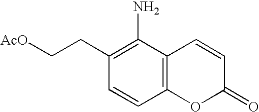

saponifying the product of step f) with a suitable base in a sixth suitable solvent so as to produce the instant compound.

This invention provides the instant process wherein the brominating agent is NBS, the first suitable solvent is CCl₄, the second suitable solvent is DMSO, the second suitable solvent is THF, the reducing agent is BH₃, the fourth suitable solvent is Ac₂O, the suitable acid is H₂SO₄, the suitable acid nitrating agent is fuming HNO₃, the suitable catalyst is PtO₂, the fifth suitable solvent is EtOH/EtOAc, the sixth suitable solvent is MeOH/H₂O, the suitable base is K₂CO₃.

This invention provides a process for producing the instant compound comprising:
a) exposing a compound having the structure:

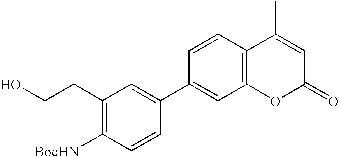

to Dess-Martin periodinane oxidation in the presence of CH₂Cl₂ so as to produce a compound having the structure:

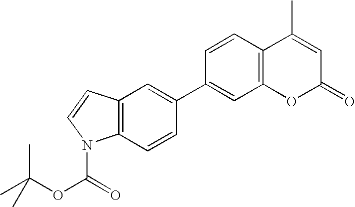

and a compound having the structure:

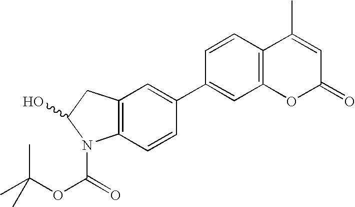

b) exposing either of both of the products of step a) to TFA in CH₂Cl₂ so as to produce the instant compound.

This invention provides a process for producing the instant compound comprising:
a) exposing a compound having the structure:

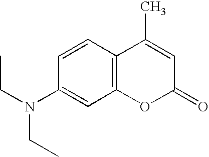

to a suitable brominating agent in the presence of a first suitable solvent so as to produce a compound having the structure:

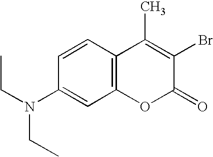

b) coupling the product of step a) with a compound having the structure:

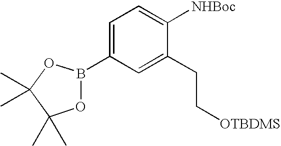

in the presence of a suitable catalyst and a suitable base in dioxane so as to produce a compound having the structure:

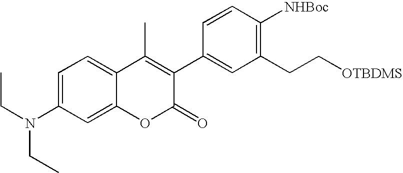

c) deprotecting the product of step b) with TBAF in THF and then TFA in CH₂Cl₂ so as to produce the instant compound.

This invention provides the instant process for producing the instant compound wherein the first suitable solvent is CH₃CN, wherein the suitable brominating agent is NBS, the suitable base is K₃PO₄, and/or the suitable catalyst is PdCl₂ (dppf).

This invention provides a process for producing the instant compound comprising:
a) exposing a compound having the structure:

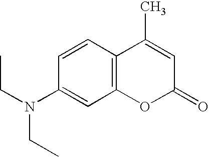

to a suitable brominating agent in the presence of a first suitable solvent so as to produce a compound having the structure:

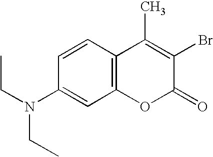

b) coupling the product of step a) with a compound having the structure:

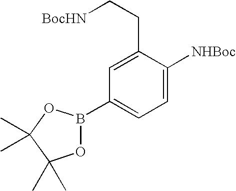

in the presence of a suitable catalyst and a suitable base in dioxane so as to produce a compound having the structure:

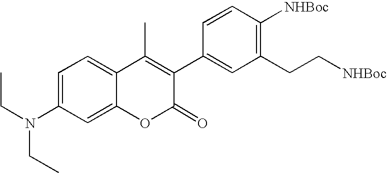

c) deprotecting the product of step b) with TFA in CH₂Cl₂ so as to produce the instant compound.

This invention provides the instant process wherein the first suitable solvent is CH₃CN, the suitable brominating agent is NBS, the suitable base is K₃PO₄, and/or the suitable catalyst is PdCl₂(dppf).

This invention provides a process for producing the instant compound comprising:
a) exposing a compound having the structure:

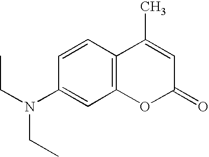

to a suitable brominating agent in the presence of a first suitable solvent so as to produce a compound having the structure:

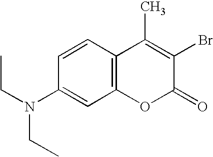

b) coupling the product of step a) with a compound having the structure:

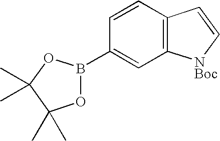

in the presence of a suitable catalyst and a suitable base in dioxane so as to produce a compound having the structure:

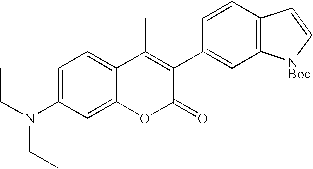

c) deprotecting the product of step b) by heating at a suitable temperature so as to produce the instant compound.

This invention provides the instant process wherein the first suitable solvent is CH₃CN, the suitable brominating agent is NBS, the suitable base is K₃PO₄, the suitable temperature is about 180° C., and/or the suitable catalyst is PdCl₂(dppf).

This invention provides a method of identifying an active MAO in a sample comprising:
providing a sample derived from a mammal;
contacting the sample with a compound which undergoes a detectable change in fluorescence when oxidatively deaminated in the presence of a MAO under conditions permitting oxidative deamination of the compound in the presence of the MAO;
detecting a change in fluorescence of the compound;
wherein an increase in the fluorescence in step c) indicates an active mammalian MAO in the sample.

This invention provides the instant method wherein the detectable change in fluorescence is a change in fluorescence emission maxima, and further provides wherein the detectable change in fluorescence emission maxima is a shift to a longer wavelength.

This invention provides a method of identifying method identifying a test compound as an inhibitor of MAO in a solution comprising:
providing a solution comprising the MAO;
contacting the solution with a compound which undergoes a detectable change in fluorescence when oxidatively deaminated in the presence of a MAO under conditions permitting oxidative deamination of the compound in the presence of the MAO;
quantitiating the fluorescence of the compound in the solution and quantitiating any H₂O₂ production in the solution;
contacting the sample with the test compound;
quantitating the fluorescence of the compound in the solution in the presence of the test compound and quantitiating the H₂O₂ production in the solution in the presence of the test compound;
wherein a decrease in the fluorescence quantitiated in step e) as compared to the fluorescence quantitiated in step c) and a decrease in H₂O₂ production quantitiated in step e) as compared to step c) indicates that the test compound is an inhibitor of the MAO.

This invention provides the instant method, wherein the production of H₂O₂ is quantitiated chemically or by fluorescence.

This invention provides a method identifying a test compound as a substrate of MAO in a solution comprising:
providing a solution comprising the MAO;
contacting the solution with a compound which undergoes a detectable change in fluorescence when oxidatively deaminated in the presence of a MAO under conditions permitting oxidative deamination of the compound in the presence of the MAO;
quantitiating the fluorescence of the compound in the solution and quantitiating any H₂O₂ production in the solution;
contacting the sample with the test compound;
quantitating the fluorescence of the compound in the solution in the presence of the test compound and quantitiating the H₂O₂ production in the solution in the presence of the test compound;
wherein a decrease in the fluorescence quantitiated in step e) as compared to the fluorescence quantitiated in step c) and no change or an increase in the H₂O₂ production quantitated in step e) as compared to step c) indicates that the test compound is a substrate of the MAO.

This invention provides the instant methods wherein the production of H₂O₂ is quantitiated chemically or by fluorescence, wherein the monoamine oxidase is MAO-B, wherein the monoamine oxidase is MAO-A, wherein the detectable change in fluorescence is a change in fluorescence emission maxima, and wherein the detectable change in fluorescence emission maxima is a shift to a longer wavelength.

This invention provides the instant methods wherein the fluorescence emission maxima is measured at about 460 nm to about 560 nm under conditions comprising excitation of the compound at about 275 nm to about 390 nm, or wherein the fluorescence emission maxima is measured at about 425 nm to about 650 nm under conditions comprising excitation at about 300 nm to about 420 nm of the compound.

This invention provides the instant method, wherein the known compound has the structure:

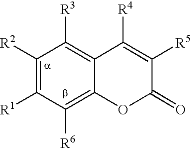

wherein R¹ is —H, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical,
or R¹ and R² form an unsubstituted pyrrole,
or R¹ and R⁶ form an unsubstituted pyrrole,
or R¹ forms an octahydro-quinolizine with R² and R⁶,

wherein each of R², R³, R⁴, R⁵, or R⁶ is independently —H, —OH, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, halide, indole radical, or R² and R³ form a pyrrole,

wherein when R¹ and R² form a pyrrole, a N of the pyrrole is covalently bound to carbon α,

wherein when R¹ and R⁶form a pyrrole, then R⁴ is —H,

wherein when R¹ is —N(alkyl)₂ or —NH₂, then R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,
wherein when R¹ is H, R³ is —NH₂ and R² is H; or R² and R³ form a pyrrole; or R² is —NH₂ then R⁴ is alkyl,
wherein when R¹ is CH₃, R² is —H, —OH, alkyl, alkenyl, alkynyl, substituted or unsubstituted aryl, cycloalkyl, substituted or unsubstituted heteroaryl, —NH-alkyl, —N(alkyl)₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical, and R¹ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,
or a salt or stereoisomer thereof.

This invention provides the instant method, wherein the known compound has one of the structures set forth below:

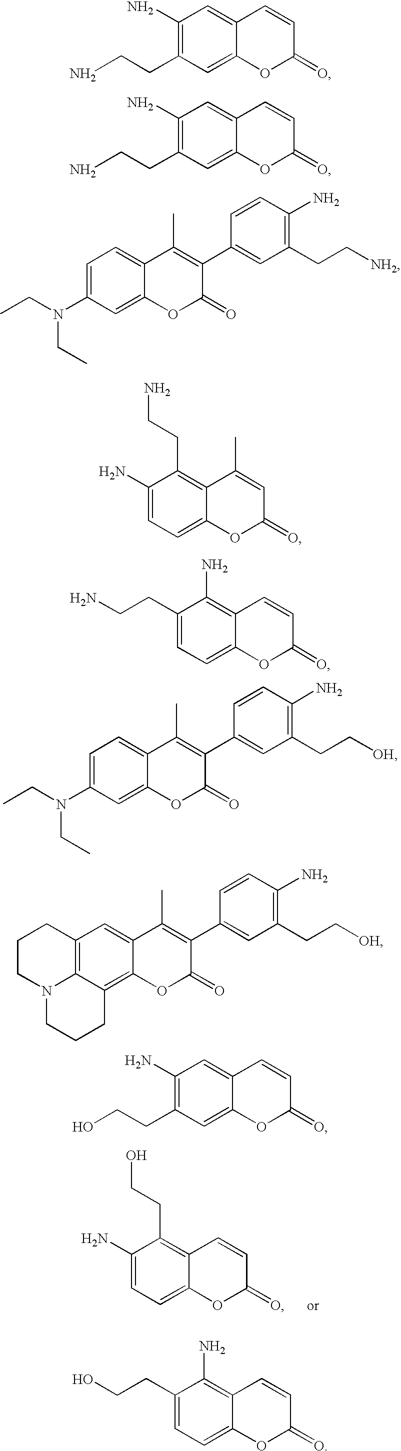

This invention will be better understood by reference to the Experimental Details which follow, but those skilled in the art will readily appreciate that the specific experiments detailed are only illustrative of the invention as described more fully in the claims which follow thereafter.

Experimental Details

First Series of Experiments

Note that probe numbers for the first series of experiments to not correspond with probe numbers for the second series of experiments.

Syntheses of Type I Probes

The intervening π-system between the host fluorophore and the donor part is a very important aspect of probe design, since it is this linkage that will define the strength of the photo-physical perturbation of the fluorophore by the reported chemical transformation. A critical lesson learned from preliminary results is that the relative arrangement of the donor group and the fluorophore plays an important role in enabling the probe to act as an enzyme substrate. In practice, three issues need to be addressed: to which position of the coumarin core the donor part should be attached; to which position of the donor part should coumarin core be used to be attached, and what linker should be used between them.

In theory, positions 3, 4, 5, 6, 7 and 8 of coumarin scaffold are all potential sites for substitution (Scheme 1). Substituents introduced on positions 3 and 7 have relatively stronger electronic effects on the optical properties of the resultant molecules than do substituents on others. It was envisaged that position para to the amino group would have the most pronounced sensitivity to the electronic change associated with the aniline-indole conversion. The donor part can be attached to the coumarin core via a linker of variable length and shape. For the sake of simplicity, the two components were attached directly through a carbon-carbon bond in our first model.

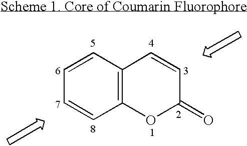

Although various synthetic routes to coumarins, especially to simple derivatives, are known, much effort is still being devoted to exploring newer methods because the existing methods lack generality and/or efficiency. Pd-catalyzed cross coupling reactions seem to be able to provide a fast and convenient method towards this goal. However, those types of reactions involving coumarins have not been amply reported. The efficiency of Suzuki, Sonogashira, Stille, and Heck-type coupling reactions were thus been explored.

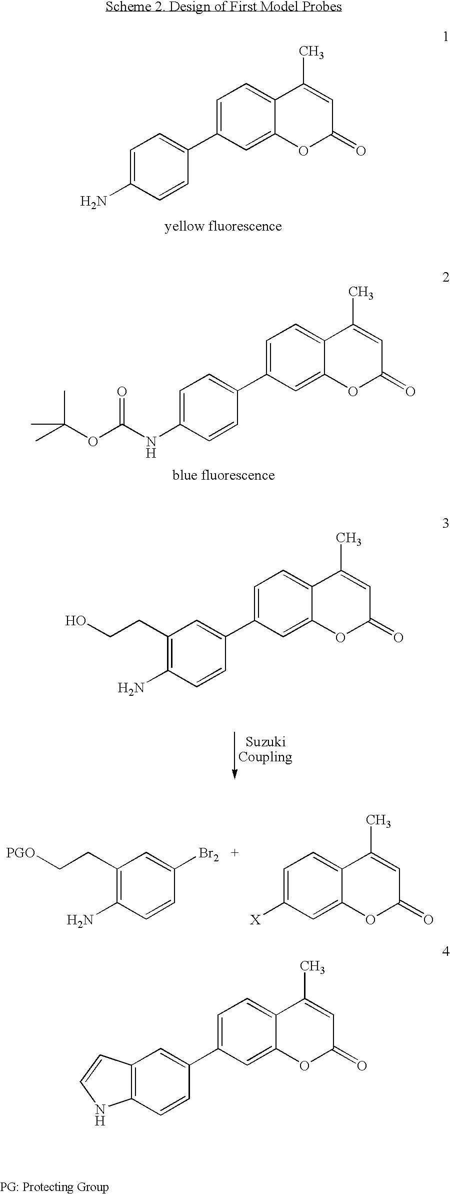

In a separate effort to develop fluorescent probes for assaying NAD(P)H, coumarin derivatives 1, 2 were synthesized (Scheme 2). Under UV irradiation, compound 1 with a free amino group shows yellow fluorescence and compound 2 with the amino group protected by carbamate shows blue fluorescence. It was believed that altered electron donating ability of the amino group has effected the fluorescence emission shifting. It was then anticipated that our first model probe based on this structural motif could be designed. Synthesis of the probes is outlined in Scheme 9; Pd-catalyzed Suzuki coupling furnished a convergent synthesis with an aryl-aryl bond formation.

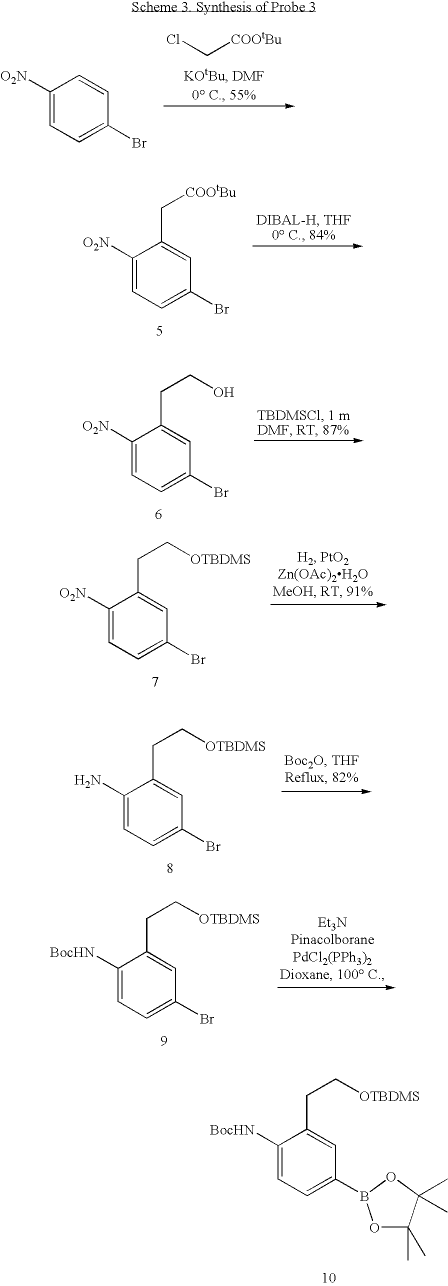

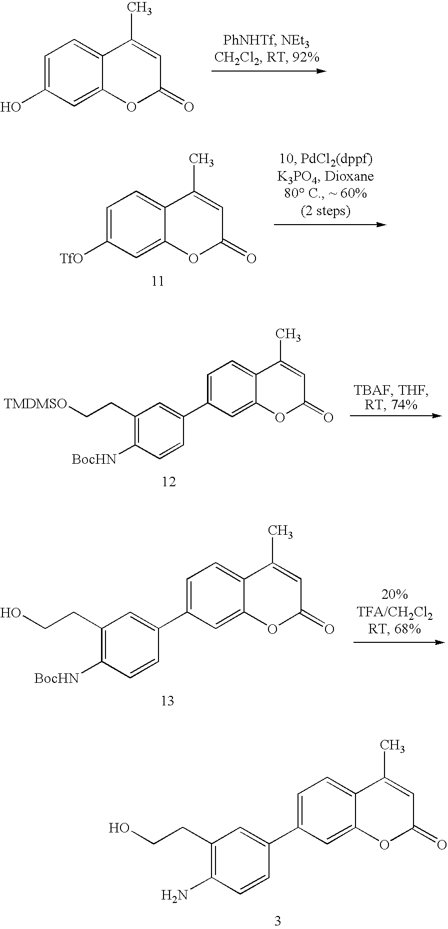

Compound 5 was prepared through a so-called vicarious nucleophilic substitution of hydrogen of para-bromo-nitrobenzene with tert-Butyl α-chloroacetate using KO^{t}Bu as base (Makosza, M. Chimia 1994, 48:499-500; Katayama, S., Ae, N., Kodo, T., Masumoto, S., Hourai, S., Tamamura, C., Tanaka, H., Nagata, R. J. Med. Chem. 2003, 46:691-701). Reduction of the ester with DIBAL-H gave alcohol 6, which was then protected by TBDMS. Catalytic hydrogenation of compound 7 with PtO₂/Zn(OAc)₂, followed by Boc protection of the amino group, furnished compound 9. Pd-catalyzed coupling of pinacolborane with compound 9 yielded the arylboronic ester 10 in good yield. The coumarin coupling partner 11 was prepared by the triflation of 7-hydroxy-4-methyl-coumarin. With the two advanced intermediates 10 and 11 in hand, the stage was then set for the key Suzuki coupling reaction. Standard conditions with PdCl₂(dppf) as catalyst and K₃PO₄ as base delivered compound 12 in good yield. Following deprotection of TBDMS and Boc, the final probe 3 was obtained.

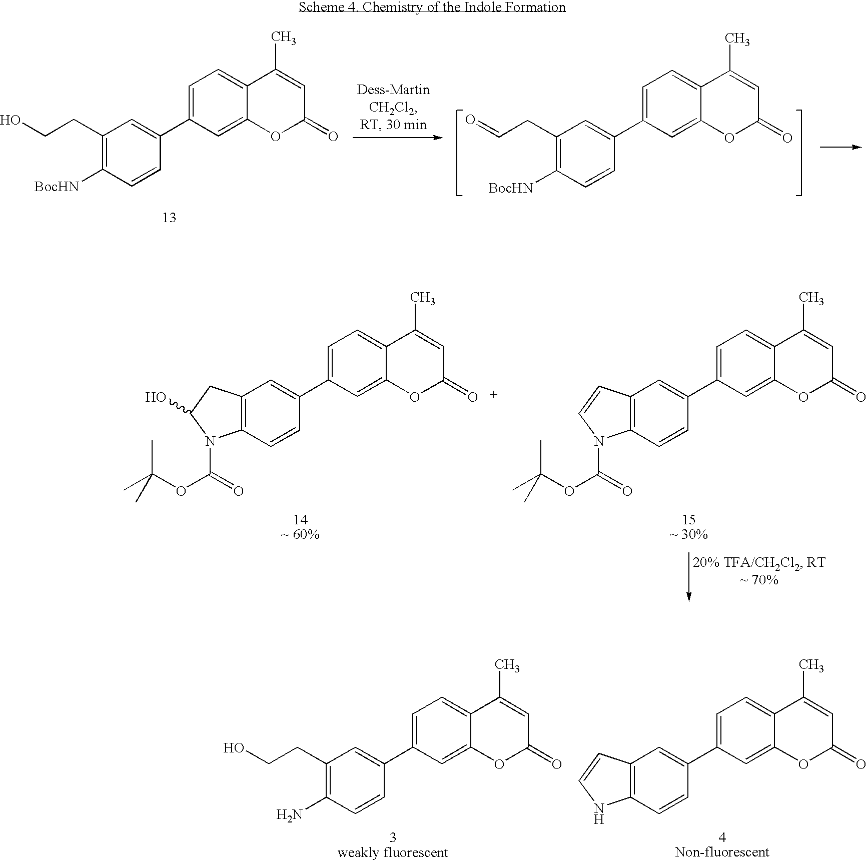

The next question was whether oxidation of the alcohol to the aldehyde would lead to the desired rapid indole ring formation and concomitant fluorescence emission shift. This can be addressed by testing selective oxidation of the probe 3. The compound 13, whose amino group was protected with Boc, was used for the purpose since free aniline might be oxidized under the oxidation conditions. Thus compound 13 was submitted to Dess-Martin periodinane oxidation, which is a known mild alcohol-to-aldehyde protocol. The reaction proceeded smoothly to completion in 30 minutes at room temperature in CH₂Cl₂ solution. Two major products 14 and 15 were isolated at the end of the reaction in ˜60% and ˜30% yield, respectively. No aldehyde intermediate was detected by TLC during the reaction. This showed that the aldehyde, once formed, was rather reactive and underwent rapid intramolecular condensation even with the carbamate protected amino group. The rapid condensation is crucial to the success of our probe design since it is based on a cascade reaction pathway where the monitored oxidation needs to the rate-determining step. Both compound 14 and 15 can be converted into the final product 4 upon exposure to 20% TFA in CH₂Cl₂ at room temperature.

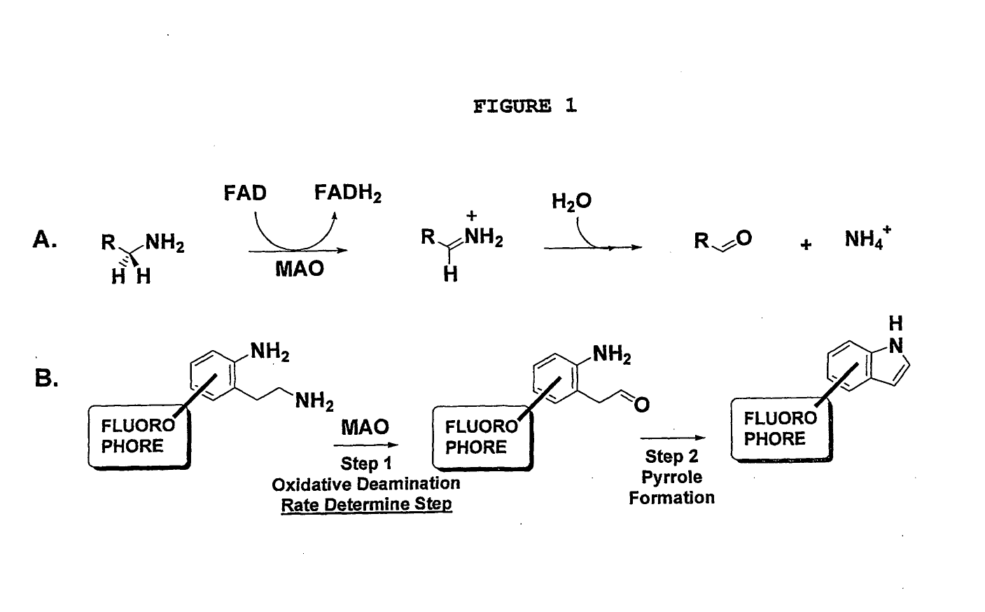

Both probe 3 and 4 were chemically stable and considerably soluble in aqueous solution. Their fluorescence spectra are presented in FIG. 1. Probe 3 shows weak fluorescence in phosphate buffer. However probe 4 is almost non-fluorescent in the same buffer solution.

Although the first probes failed to fulfill the fluorescence switch criteria, the expected cascade transformations of oxidation and indole formation are favorable. Secondly, this cascade does lead to distinct fluorescence readout, although, in the first case, the fluorescence switched in an undesirable direction.

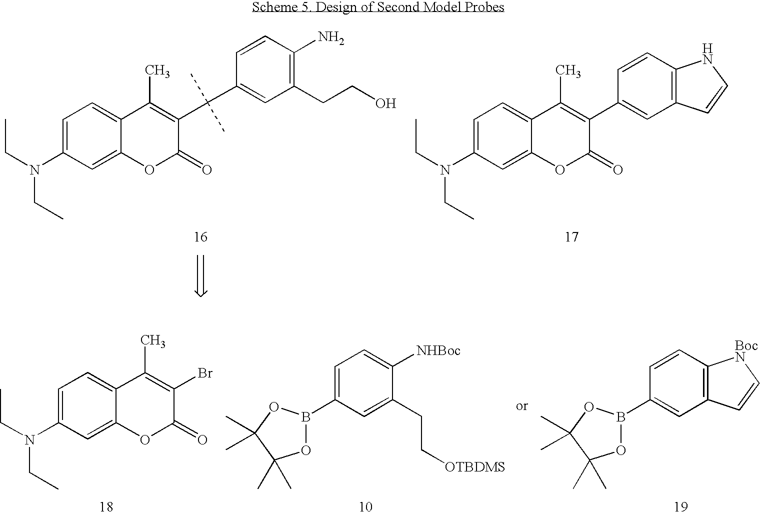

It was realized that an electron-donating group at position 7 of coumarin skeleton is important to the fluorescent quantum yield. It is known that N,N-diethylamino-4-methylcoumarin is an excellent laser dye. By keeping the diethylamino group at position 7 to maintain its fluorescence and attaching the reporting part at position 3, probes 16 and 17 were designed as our second models, as shown in Scheme 5.

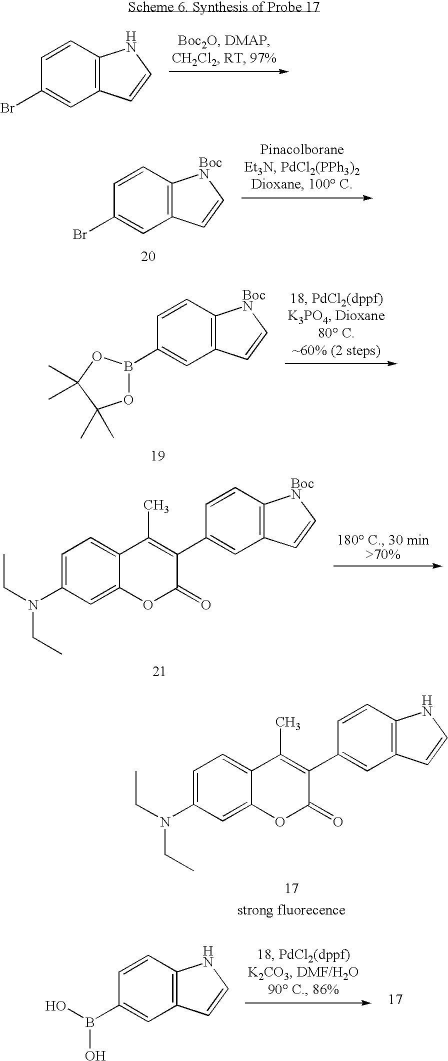

Similar retro-synthesis as that of probe 3 was applied. One lesson learned from the first probe design was that it would be wise to make the indole probe 17 and check its fluorescence first before pursuing probe 16. The synthesis is outlined in Scheme 6. 6-bromo-indole was protected with Boc and then converted to the corresponding boronic ester 19 using the same conditions as used for probe 10. Suzuki coupling of 19 and 10 afforded 21 in good yield. Treatment of 21 with 20% TFA in CH₂Cl₂ gave probe 17 in poor yield presumably because of decomposition of product under strongly acidic conditions. It was found, however, that thermal deprotection of 21 at 180° C. over 30 minutes yielded 17 in excellent yield. Alternatively, it was found that 17 could be straightforwardly prepared by direct coupling of indolyl boronic acid and 18. Probe 17 was found to be highly fluorescent.

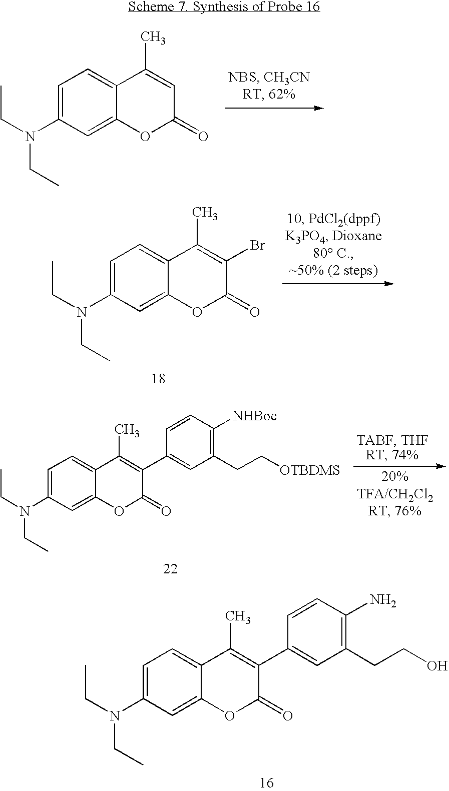

Probe 16 was then prepared in a similar manner as probe 3 (Scheme 7). Commercially available N,N-diethyl 7-amino-coumarin was selectively brominated with NBS in CH₃CN to give compound 18. Suzuki coupling, deprotections of TBDMS and Boc furnished probe 16 in good overall yield.

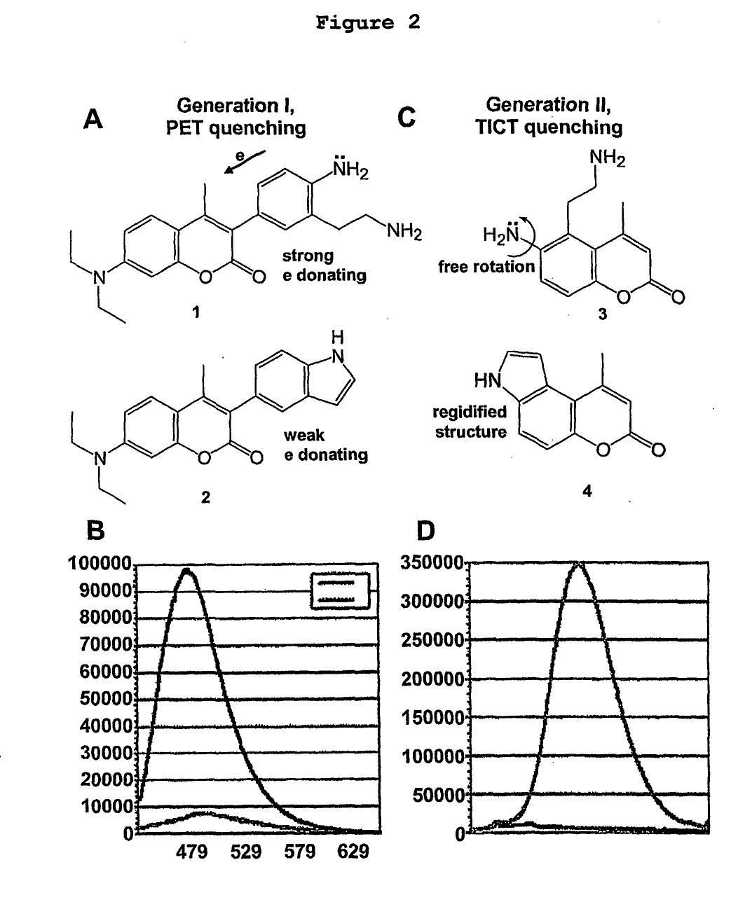

Probes 16 and 17 were chemically and photo-physically stable, and reasonably soluble in aqueous solution. Most importantly, probe 16 was much less fluorescent than Probe 17 (FIG. 2). The indole 17 possesses 12-fold fluorescence emission intensity of the aniline 16. The maximum emission wavelength is over 480 nm, which should be suitable for future in vivo assays and imaging.

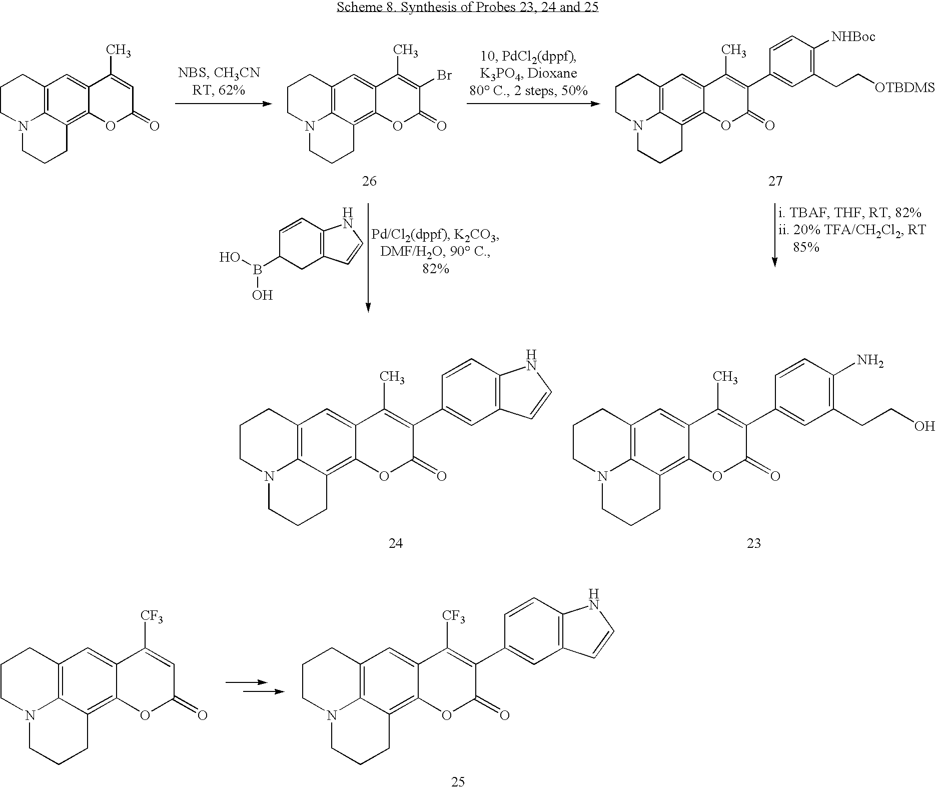

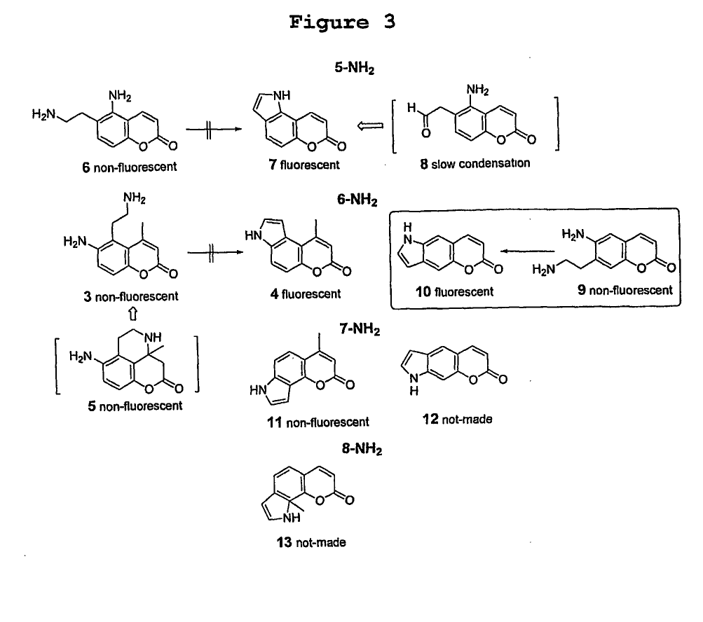

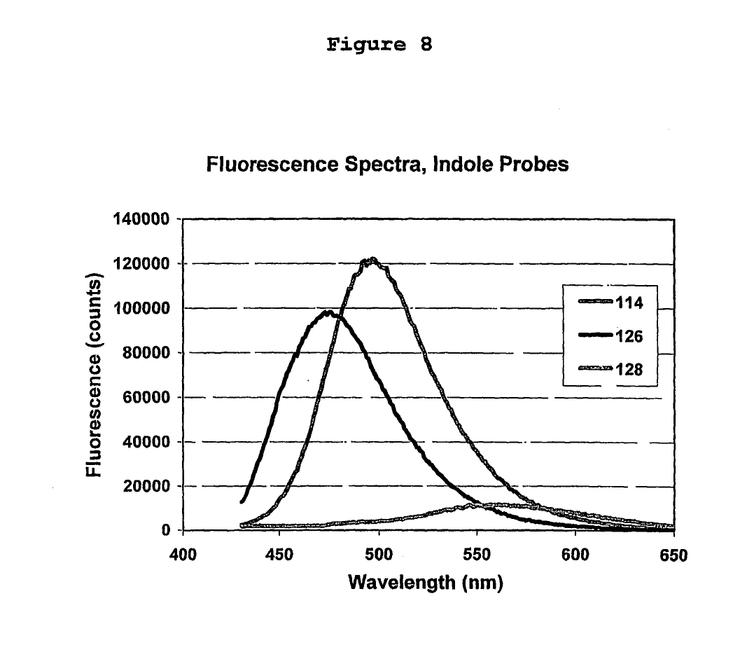

In order to compare probes with different structural and fluorescent properties, analogs of 16: 23, 24 and 25, were prepared using similar chemistry (Scheme 8). Fluorescence spectra of probes 23 and 24 are shown in FIG. 3. Probe 24 emits at 495 nm more intensely than probe 17 does at 475 nm. But probe 23 has higher background fluorescence and has only 6-fold fluorescence emission increase when oxidized to 24. As shown in FIG. 8, Probe 25, prepared by simply replacing 4-methyl with 4-trifluomethyl, has a distinct red-shift emission. Unfortunately, it has a rather low quantum yield, which seriously diminishes its sensitivity as a reporter substrate. Among probes 17, 24, and 25, 17 has the most distinct fluorescence increase and lowest background signal. Compared to 24, it also has a smaller size, which may make it more susceptible to enzymatic oxidation.

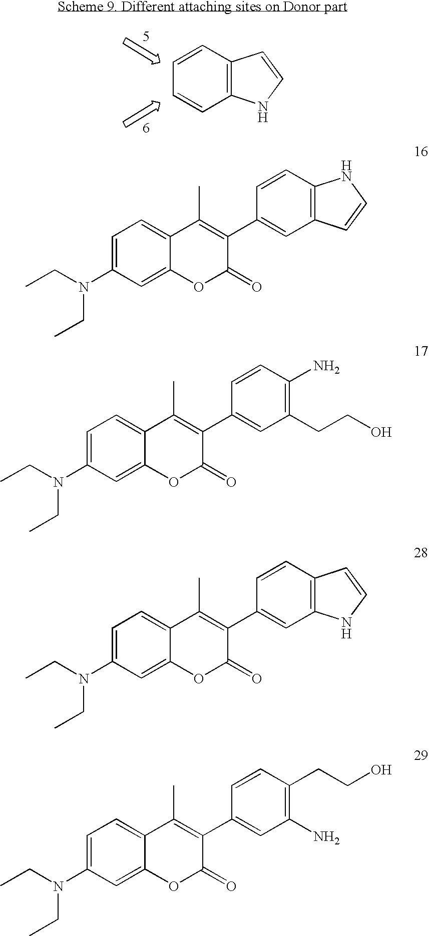

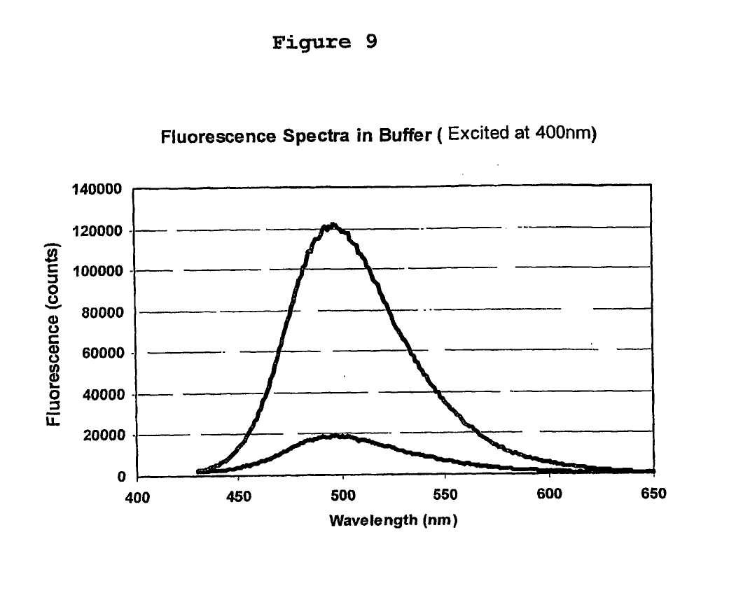

So far, we have studied probes with an aniline moiety attached at positions 3 and 7 of the coumarin core. Attachment through position 7 gave us the desired fluorescence switch. The other question that needs to be answered is whether different attachment sites on the donor aniline moiety could also affect the fluorescence switch (Scheme 9). Positions 5 and 6 of the indole moiety appear to be the most sensitive positions for electronic modifications. Therefore, probe 28 was designed and synthesized (Scheme 10). The fluorescence spectra of probes 17 and 28 were shown in FIG. 9.

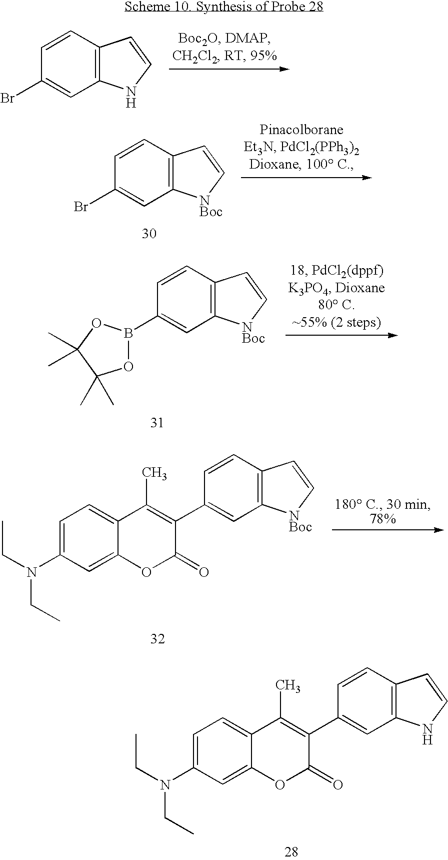

Probes 17 and 28 were found to have similar fluorescence quantum yields. We hypothesized that an amino group para to position 3 of coumarin probe 16 might have a stronger electron donating effect and therefore better quenching ability. It was also envisioned that probe 16 would have a weaker background emission than probe 29. Although probe 29 was not synthesized, we expected that probes 16 and 17 would have better fluorescence switching ability than probes 28 and 29.

So far, our fluorescence switch design was tentatively built on a simplified electronic argument: PET quenching. Two experimental facts support the argument that the quenching is indeed due to electron transfer from the PET donor to the excited fluorophore. First, only small alterations in absorbance were observed between the aniline and the indole forms. Second, the dihedral angle between the aniline moiety and the coumarin plane is about 96°, as found by MM3 calculation, which suggests that there is little ground-state interaction between these two parts. In theory, we could achieve a better fluorescence switch by fine-tuning the two components through extensive chemical modifications.

Synthesis of Type II Probes

To further improve signal to noise ratio and to sterically reduced the structures of the probes, new designs of different structural features and fluorescent properties were investigated. To this end, another type of probe designs based on different structural motifs and even different quenching mechanisms was developed.

It is known that coumarin dyes that possess an electron donor substituent at position 7 show rather high fluorescence quantum yields. The fact that a donor substituent at position 6 strongly reduces the fluorescence quantum yield of coumarins has not captured too much attention. It was reported that the fluorescence of 6-aminocoumarin is very weak, and its photo-physical data is consistent with the emission from a twisted intramolecular charge transfer (TICT) state. In that state, the amino lone-pair axis is twisted into the plane of the coumarin skeleton, perpendicular to the coumarin π-orbitals. The increased dipole moment from the twisted geometry provides the driving force for the twist towards the TICT (90° C.) conformation with a maximum charge separation via the polar solvent induced energy stabilization.

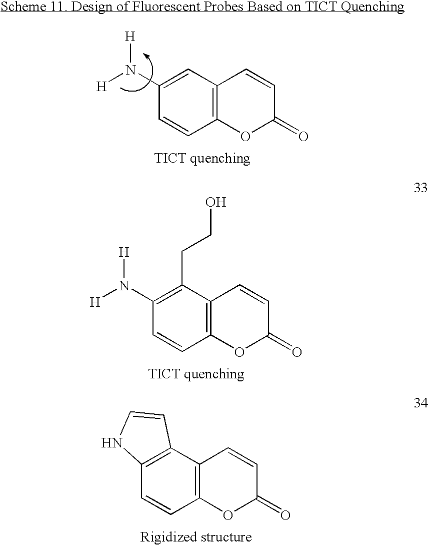

New probes were designed by installing a side chain at the ortho position of the amino group as shown in Scheme 11. The sequential alcohol oxidation, intramolecular condensation, and isomerization would lead to the formation of 34. Obviously, two important issues needed to be explored: one, whether probes 33 and 34 have the desirable fluorescence switch; and two, whether the cascade reactions would proceed rapidly. Our working hypothesis was that probe 33 would be weakly fluorescent due to the TICT quenching and probe 34 may be fluorescent due to the rigidized structure's preventing the C—N rotation. This design is particularly attractive since probe 33 might have a low background fluorescent signal, and since probes of this medium size look more like naturally occurring small-molecule metabolites.

Efforts were directed to the development of fluorescent probes wherein a measurable change in emission properties (wavelength, intensity) of the probe occurs during the chemical transformation. However, a fluorogenic probe such as probe 33, which simply changes its fluorescence intensity with chemical transformation, tends to be affected by various factors, such as the probe concentration and environmental conditions (temperature, pH, etc). Probes undergoing shifts in emission spectra are preferable to be those that only undergo only changes in fluorescence intensities: indeed, after calibration, the ratio of the fluorescence intensities at two appropriate emission wavelengths provides a measurement independent of the probe concentration and insensitive to the intensity of incident light, scattering, and photo-bleaching.

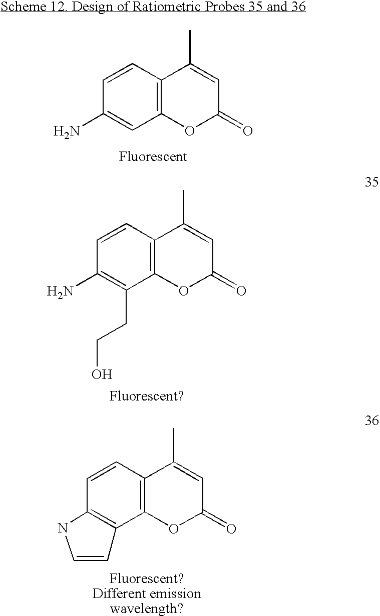

It is known that 7-aminocoumarins are highly fluorescent. It was then hypothesized that 35 with a side chain on the position 8 would form pyrrolocoumarin 36 after oxidation (Scheme 18). If the formation of fused pyrrole ring could shift the fluorescence emission wavelength of 36, 35 would work as a good ratiometric probe. The photo-physical properties of pyrrolocoumarins have not been systematically studied, in fact, few have been synthesized. To this end, the amino coumarins and their corresponding pyrrolocoumarins shown in Scheme 19 were synthesized and their fluorescence properties were systematically studied. While the structures of those probes look simple, their syntheses turned out to be not straightforward at all. In general, one of two synthetic approaches was taken: introduction of a side chain to a known coumarin precursor, or alternatively, by preparing the coumarin from a properly substituted benzene precursor. Although many methodologies have been developed for the construction of the coumarin core, chemo-selective synthesis of multi-substituted coumarins still poses a challenge. Hence, modifications of known coumarin precursors through installation of a side chain seemed to be the more efficient route.

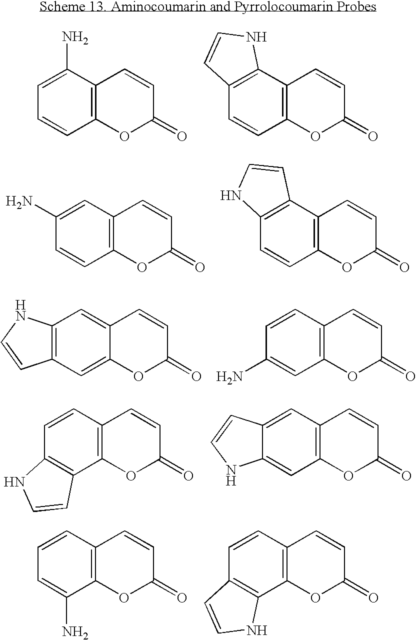

Our explorations started with the syntheses of probes 35 and 36. Two key reactions were planned to furnish 35: a Claisen rearrangement to install the allyl group at position 8 and later a Buchwald coupling for the amination at position 7. Commercially available 7-hydroxycoumarin was allylated to give 7-allyoxycoumarin 37, which underwent Claisen rearrangement to regioselectively produce 38 in respectable yield by refluxing it in N,N-diethylaniline. The free phenolic hydroxy was then triflated to give 39. The allylic double bond was cleaved with osmium tetraoxide dihydroxylation followed with NaIO₄ oxidation to yield 40, which was reduced by NaBH₄ to afford 41. The intermediate was then subjected to the Buchwald coupling with benzophenone imine to attempt to yield the diphenyl ketimine. Surprisingly only undesired product 42 was obtained. Then the hydroxy group was protected with TBDMS to give 43 and subjected to various coupling conditions with combinations of different palladium sources, ligands, bases, and solvents. They included Pd(OAc)₂, Pd₂(dba)₃, DPPF, BINAP, NaO^{t}Bu, Cs₂CO₃, THF and toluene. Despite these efforts, no noticeable amount of product 44 was formed. The attempted synthesis through a late-stage conversion of 7-hydroxy into amino failed to furnish final probe 35 also. The synthetic plan was then revised with the prerequisite that the amino group should be installed in the early stage of the synthesis.

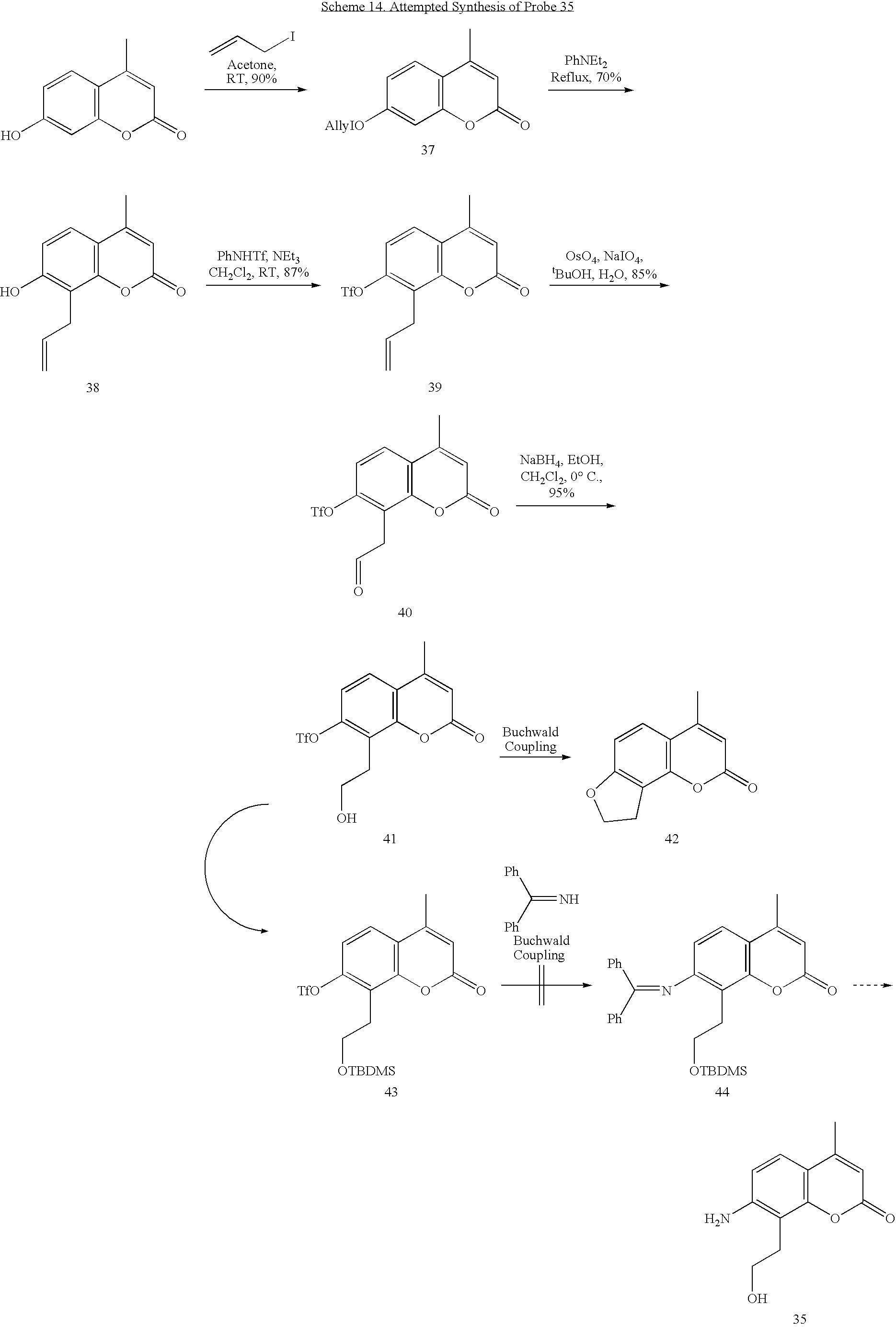

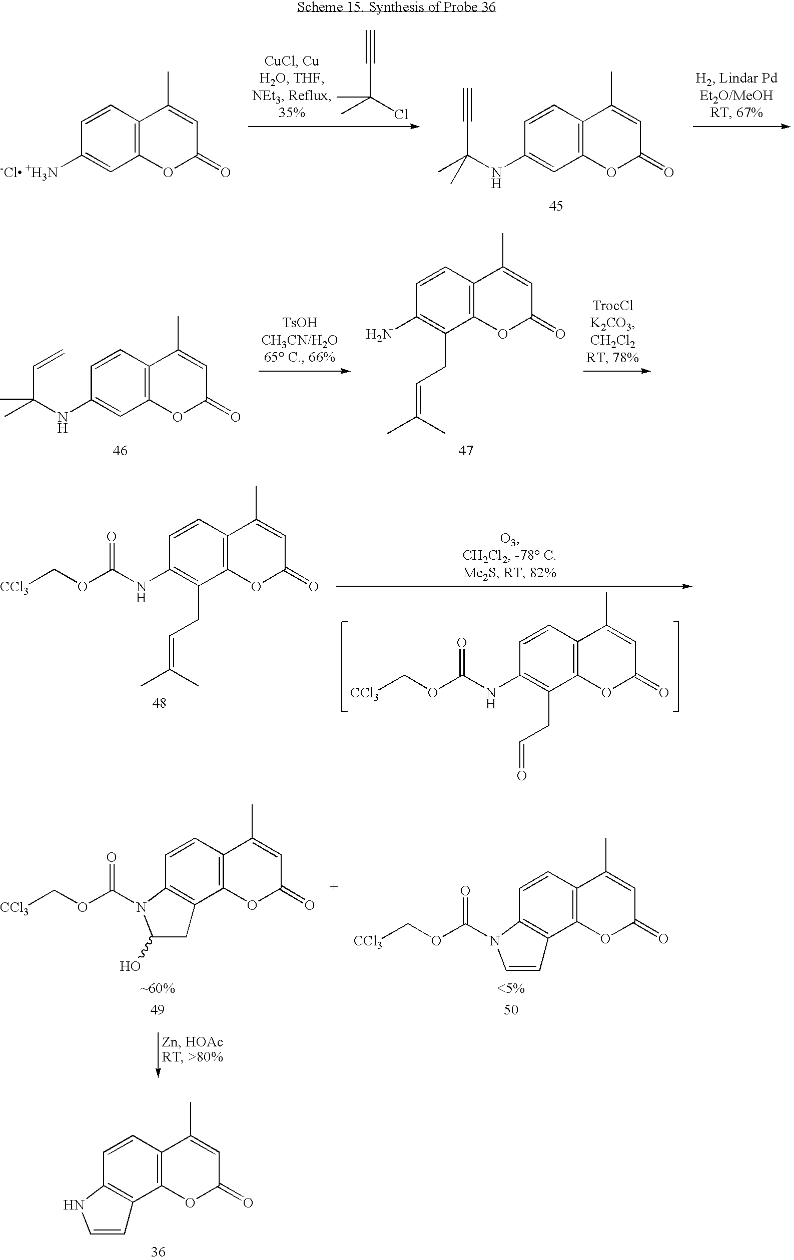

The new synthetic route is shown in Scheme 15. The key step was an amino-Claisen rearrangement. Although the Oxy-Claisen rearrangements on coumarins have been studied in detail, their amino-variants have not. It is known that the activation energy required for an amino-Claisen rearrangement is higher than that of a normal one. Ward et. al has established an efficient method for introduction of an allyl group ortho to the aniline amino group through a catalytic amino-Claisen rearrangement. They reported that aromatic amino-Claisen rearrangements of N,N-(1,1-disubstituted-allyl)anilines are facile in aqueous acetonitrile at 65° C. using 10 mol % para-toluenesulfonic acid monohydrate as the acid catalyst.

The requisite 1,1-dimethylallyl amine 46 can be readily prepared from the commercially available 7-amino-4-methylcoumarin. Cu-catalyzed coupling of aminocoumarin with 3-chloro-3-methyl-1-butyne gave propargylamine 45, which was converted to allylamine 46 by partial hydrogenation using the Lindlar catalyst. The rearrangement was conducted according to the reported condition; excellent yield and stereo-selectivity were achieved. Compound 47 was the predominant product with only trace amounts of other isomers. Coumarin derivatives are generally stable to acid but not to base. Therefore, most of our chemical conversions were conducted in acidic conditions. The free amino released from the rearrangement need to be protected. Troc was used here because this protecting group can be easily put onto the aniline amine by reacting with TrocCl and can be conveniently removed with zinc in acetic acid. Ozonolysis of compound 48 afforded two products 49 and 50 with no aldehyde intermediate isolated. We were pleased to observe that even the carbamate-protected amine had a sufficient nucleophicility to react rapidly with the aldehyde intermediate. Treatment of compound 49 with acetic acid led to the formation of probe 36.

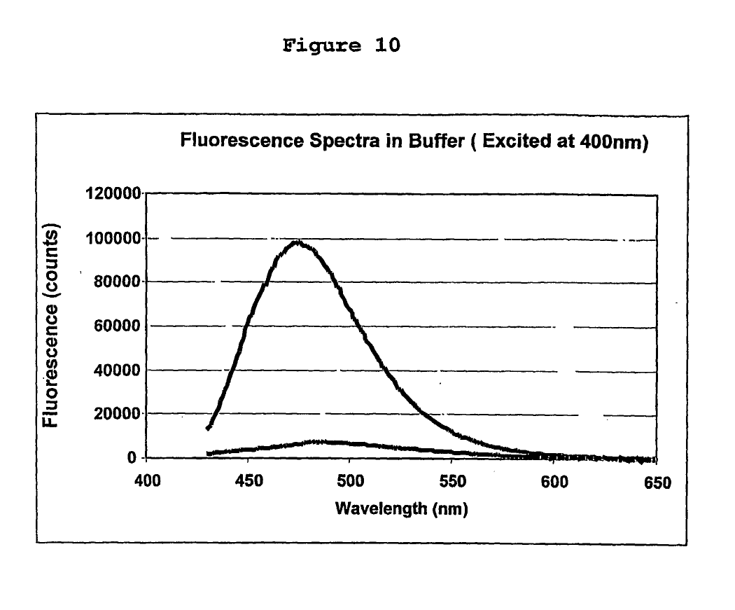

The fluorescence property of probe 36 was then evaluated (FIG. 10). Unfortunately, the very weak fluorescence emission intensity of 36 precluded it from being a sensitive fluorescent probe, let alone a ratiometric one. Attaching substituents at different positions of the host fluorophore can result in considerably different fluorescence properties. At the current stage, it is still rather difficult to predict them rationally. In order to improve efficiency in our exploration of the structure-fluorescence relationship between aminocoumarins and pyrrolocoumarins, we need to develop fast, reliable syntheses of pyrrolocoumarins and test their fluorescence properties before synthesizing the aminocoumarins.

In view of this, we planned to prepare pyrrolocoumarins without any substituent on the pyrrole ring. The easiest way to make the pyrrolocoumarins seemed to be the direct formation from hydroxycoumarin and ethyl propynoate. Classical Pechman conditions using concentrated H₂SO₄ as solvent at elevated temperature, Trost's conditions using Pd₂(dba)₃ as catalyst and formic acid as solvent, or Fujiwara's condition using Pd(OAc)₂ as catalyst and TFA and CH₂Cl₂ as solvents were all tried, as outlined in Scheme 16. No desired product formed under those conditions. It was assumed that these strong acid reaction conditions might be too harsh for the pyrrolocoumarin.

The Sames lab has developed a mild and neutral method for hydroarylation of arene-yne substrates using PtCl₄ as the catalyst. PtCl₄ demonstrated consistent performances with arene-yne substrates of diverse structural features, including propargyl ethers, propargylamines, and alkynoate esters, providing good to excellent yields of the cyclized products. The method was then adapted to make pyrrolocoumarins in two steps as outlined in Scheme 16.

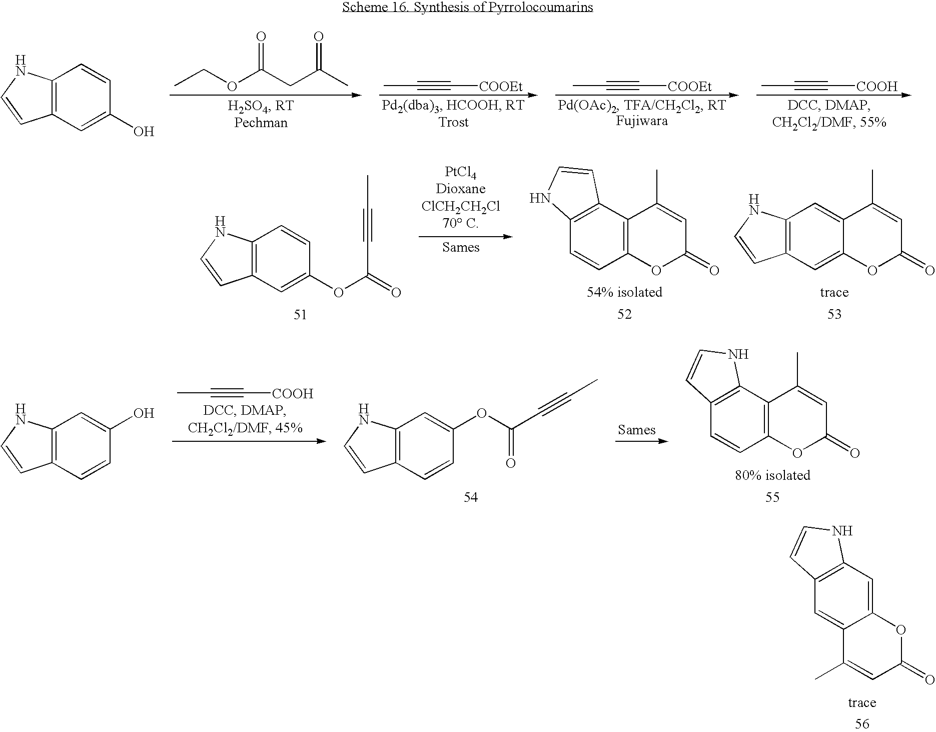

Coupling of commercially available hydroxyindole and ethyl propynoate using DCC afforded ester 51 in good yield. The ester then underwent the cyclization using PtCl₄ as catalyst and dioxane/1,2-dichloroethane (1:1) as solvent. The reaction proceeds smoothly at 65-70° C. and is complete in 3 hours. This robust reaction is found not to be sensitive to moisture or oxygen. Also, excellent regio-selectivities were achieved with both 51 and 54. The selectivities may have arisen from conservation of the aromaticity of the coumarin lactone ring.

Both 52 and 55 were quite fluorescent. It is noted that fusing porrole through positions 5 and 6 of the coumarin core does not quench its fluorescence, as opposed to those fused at positions 7 and 8.

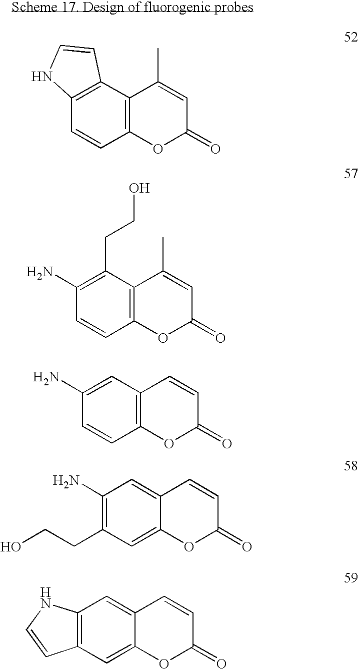

Assuming that analogs 57 and 58 of 6-aminocoumarin would have weak fluorescent emission, fluorogenic switch probes 52/57 and 58/59 shown in Scheme 23 were thus proposed. Once again the amino Claisen rearrangement strategy was utilized in the synthesis of probe 57. The correct regio-selectivity of the rearrangement reaction was expected according to the conservation of coumarin aromaticity. The route is detailed in Scheme 24. Wittig reaction of 2′-hydroxy-aetophenone with carbethoxymethylene-phosphorane in refluxing N,N-diethylaniline afforded 4-methylcoumarin 60 in excellent yield. Selective nitration of 60 by KNO₃ in concentrated sulfuric acid selectively nitrated position 6. SnCl₂.2H₂O in refluxing EtOH can reduce the nitro group, but the workup and purification was tedious. Catalytic hydrogenation should be a better choice, but the activated double bond of the lactone ring in coumarins may be hydrogenated under mild conditions, including H₂/Pd—C. Selective hydrogenation of 61 to 62 with retention of the double bond was achieved using PtO₂ as the catalyst in EtOH/EtOAc. The transformation of 62 to 65 was effected as described for the synthesis of 36.

Both aminehydrogens needed to be protected from reacting with the aldehyde intermediate. Amino group of 65 was thus protected as phthalimide. Ozonloysis of the allyl double bond and the following reduction of the aldehyde yielded 68.

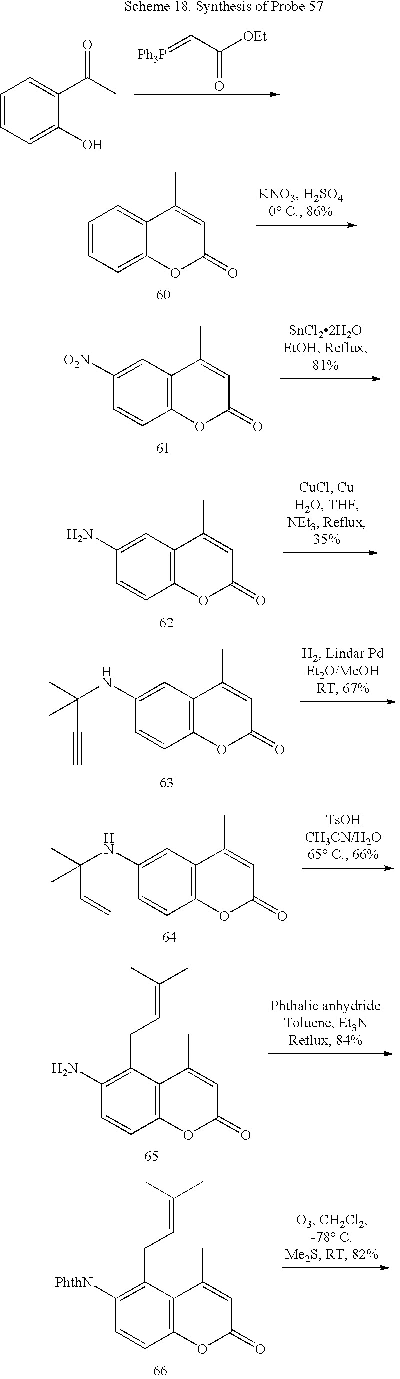

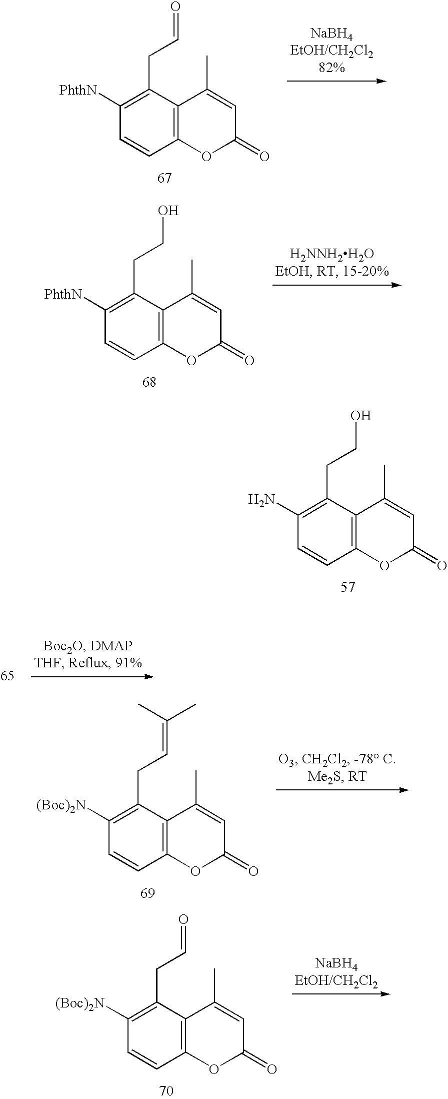

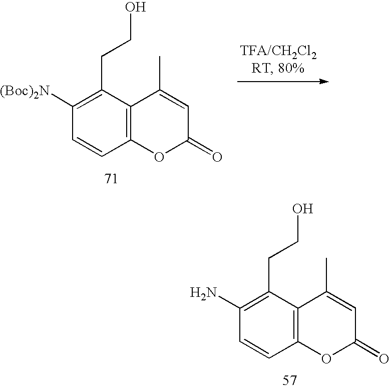

However, deprotection of phthalimide using hydrazine gave the final probe 57 in only 10-20% yield. Adjusting the reactivity of hydrazine with acetic acid showed practically no improvement in yields. Other acid labile protecting groups were tested, and we found that the amino group could be doubly Boc-protected as in 69. The deprotection of the bis-Boc 69 was carried out using 20% TFA in CH₂Cl₂, affording probe 57 in excellent yield.

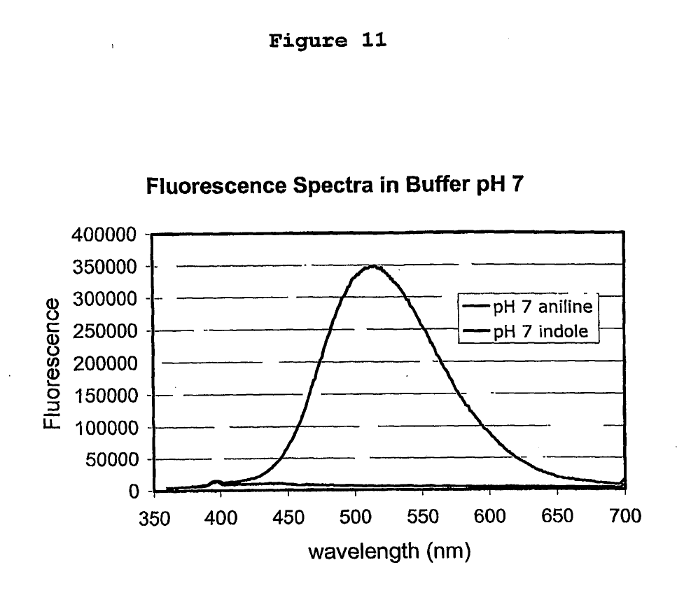

Aminocoumarin 57 possesses all the required physical and photo-physical properties of a fluorescent probe. It is chemically and photo-physically stable, and also very soluble in aqueous solution. Most importantly, it was essentially non-fluorescent while the corresponding pyrrolocoumarin was considerably fluorescent (FIG. 11).

Because of the remarkable fluorescence switch of probes 52 and 57, another pair of probes 58 and 59, were prepared. As observed in the synthesis of 57, 58 could not be prepared through the amino-Claisen rearrangement since it gave the undesired regioisomer. Two synthetic approaches are outlined in Scheme 25.

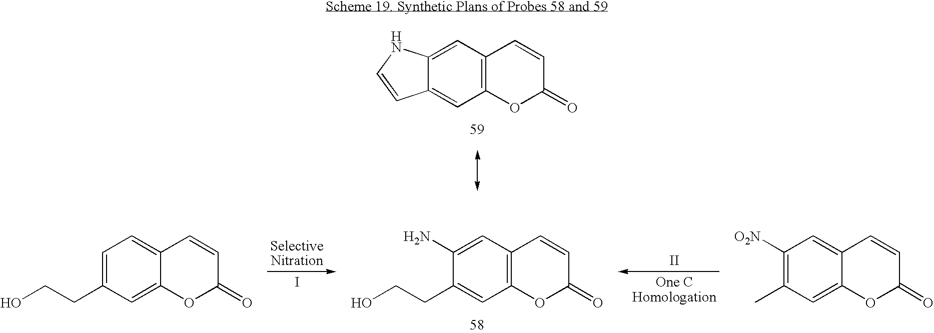

The first approach starts with the installation of the side chain, followed by the nitration/reduction sequence. The second one involves one carbon homologation of 6-nitro-7-methylcoumarin. Detailed syntheses are outlined in Scheme 19. Pd-catalyzed Stille coupling reaction of 7-triflyl-coumarin and tributylallyltin installed the allyl group at position 7 of the coumarin core almost quantitatively. Nitration with KNO₃ in cold concentrated H₂SO₄ resulted in an undesired product 73. Ozonolysis of the allyl double bond led to aldehyde 74 in surprisingly low yield. Reduction of the aldehyde with NaBH₄ afforded β-hydroxyethyl coumarin 75. Nitration of 75 with KNO₃ in concentrated H₂SO₄ resulted in its dehydration to vinylcoumarin, which is susceptible to polymerization. After screening several nitration conditions were screened, fuming nitric acid in acetic anhydride was found to successfully give the product 76 with the hydroxy protected as acetate. The nitration proceeded with great regio-selectivity and reasonable yield (˜40%). Deprotection of acetate and reduction of nitro group afforded the final probe 58. However, the low overall synthesis yield drove us to develop a more efficient alternative.

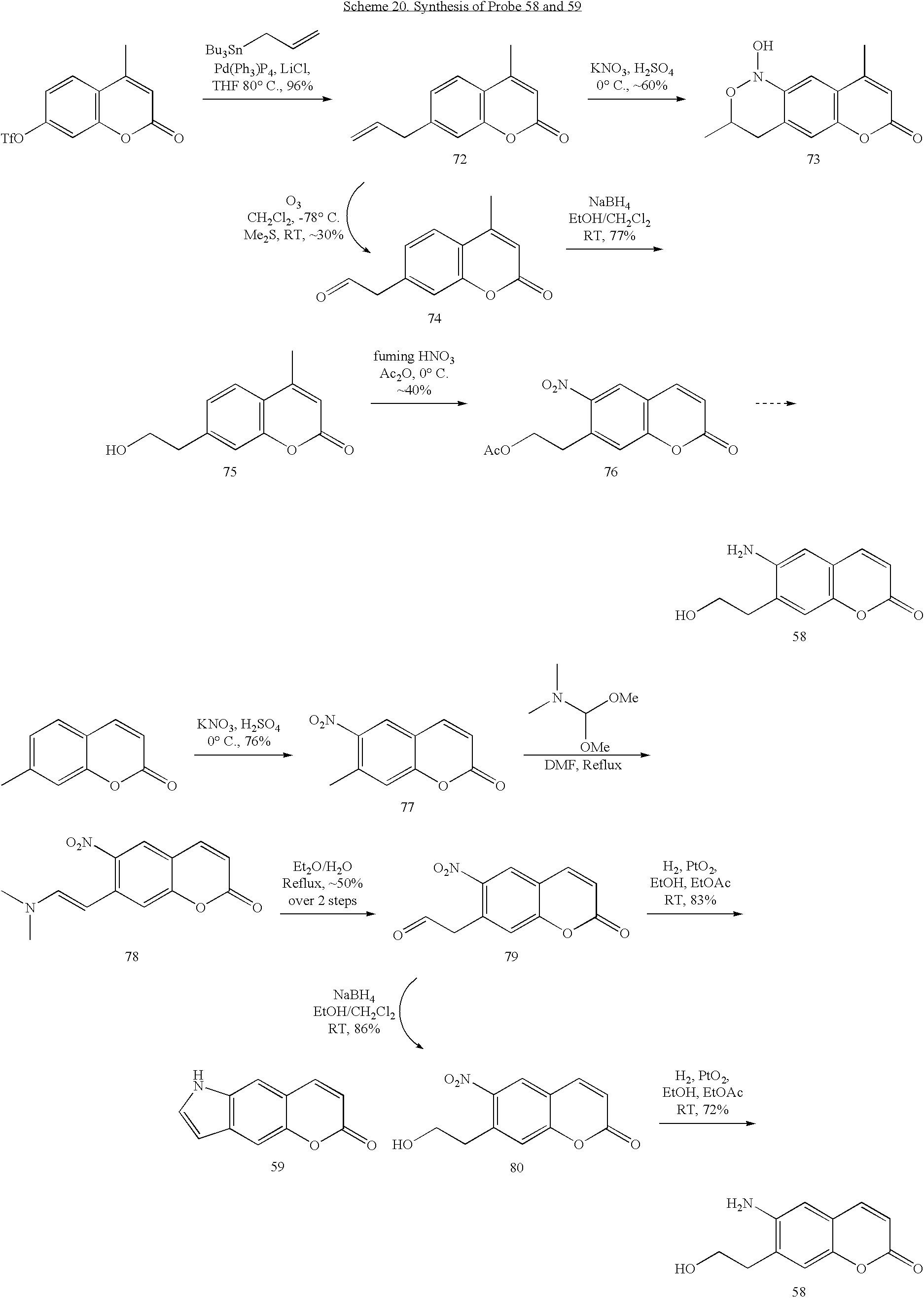

Nitration of commercially available 7-methylcoumarin afforded the desired 6-nitro-7-methylcoumarin 77 in excellent selectivity and yield. Condensation with Me₂NCH(OMe)₂ in DMF resulted in the enamine 78, which was hydrolyzed in refluxing ether and aq. HCl to afford 79 readily. Catalytic hydrogenation using PtO₂ as catalyst gave the pyrrolocoumarin 59. The aldehyde could also be reduced to yield 80, which was then converted to final probe 58 through catalytic hydrogenation. In this way, the efficiency of the synthesis was significantly improved in terms of number of steps and yields.

The fluorescence spectra of probes 58 and 59 are shown in FIG. 8. We were delighted to find that aminocoumarin 58 is basically non-fluorescent and, remarkably, pyrrolocoumarin 59 shows around 2000-fold increases in emission intensity.

Encouraged by such a spectacular fluorescence switch, we were eager to probe whether 5-aminocoumarin 81 would be similarly effective. Based on the lessons we learned from previous coumarin syntheses, four synthetic routes were proposed as outlined in Scheme 21. The Amino-Claisen rearrangement approach was thought to be reliable, but access to the intermediate 83 was found to be rather difficult. Installing the side chain by Stille coupling from intermediate 84 seemed to be a promising way, but it did not work out either. The one-carbon homologation proved efficient in the synthesis of probe 58. The same logic was thus adopted here, however, the intermediate 85 did not undergo the desired condensation presumably because of the lower reactivity of the 6-methyl group. Eventually, the synthesis was achieved by nitration of the intermediate 86. Synthetic details are shown in the following schemes.

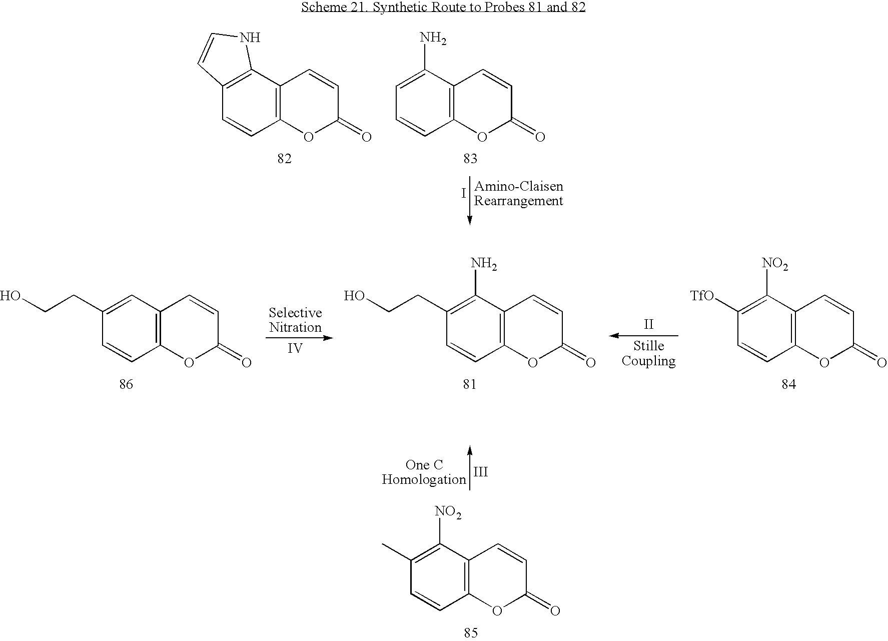

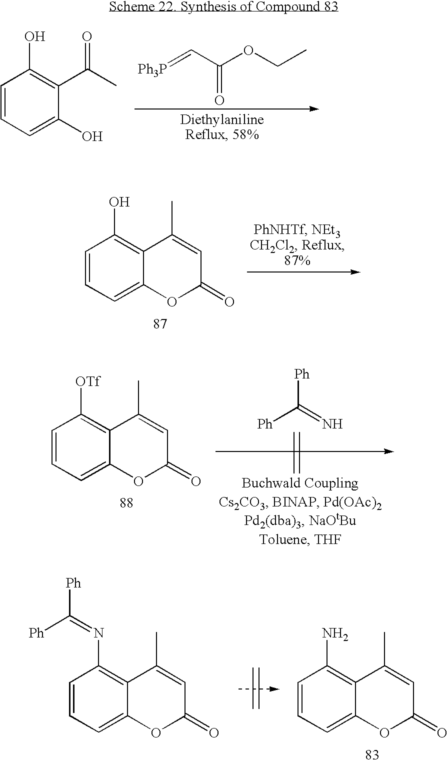

Coumarin 87 was easily prepared from 2′,6′-dihydroxyacetophenone. We assumed that the Buchwald amination of its triflate 88 could give the 5-amino-4-methylcoumarin 83. Disappointingly, this did not work despite various attempts (Scheme 22).

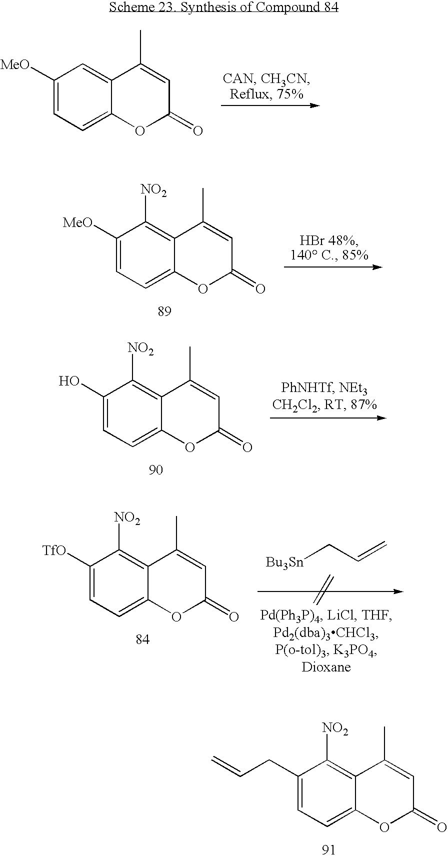

Coumarin 89 was accessible with good selectivity and yield through direct nitration of commercially available 6-methoxycoumarin using CAN in refluxing CH₃CN (Scheme 29). Deprotection of the methoxy group in boiling 48% hydrobromic acid gave compound 90, which was triflated to give the advanced intermediate 84. Surprisingly, the Stille coupling reaction, which worked well in the synthesis of compound 72, did not work at all on this substrate under various conditions tested.

The success of making probe 58 from ortho-nitro methylcoumarin inspired us to apply the same idea here (Scheme 30). Thus the nitration of 6-methylcoumarin was achieved with excellent selectivity and yield using the same conditions as in 77. It was found that the electron-donating group at position 6 of coumarin directs the nitration to position 5. However, the subsequent condensation with Me₂NCH(OMe)₂ in DMF did not give enamine 92. The lower reactivity might arise from the electron-donating property of the para ester oxygen. Other transformations, like reaction with ethyl formate and different bases, and reaction with formaldehyde under different conditions, were also unsuccessful. Bromination with NBS in refluxing CCl₄ also failed.

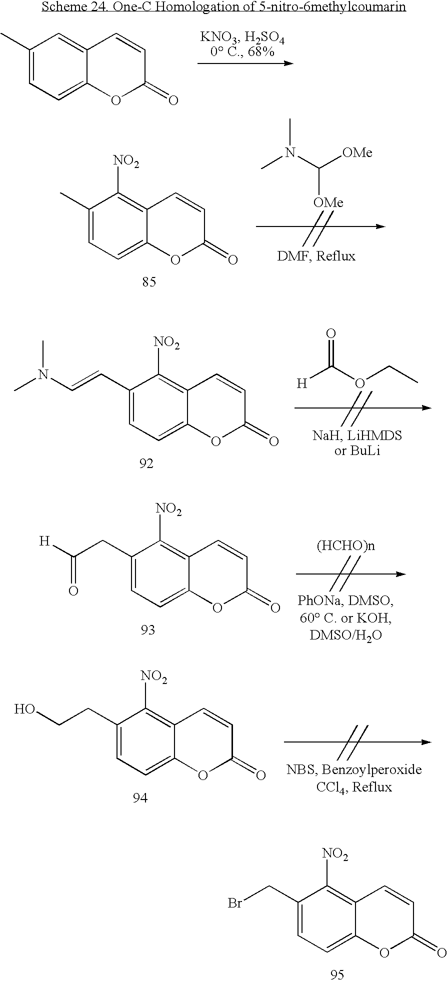

5-nitro group of 85 was then reduced to the 5-amino of 96 by catalytic hydrogenation, since this functionality seemed to be seriously hindering the reactivity of the 6-methyl group (Scheme 25). The free amino group was doubly protected with Boc to give compound 97. Bromination with NBS proceeded well, giving either mono bromide 102 or dibromide 98 with excellent selectivity and yield depending on the amount of NBS used. The dibromide was then hydrolyzed to give aldehyde 99.

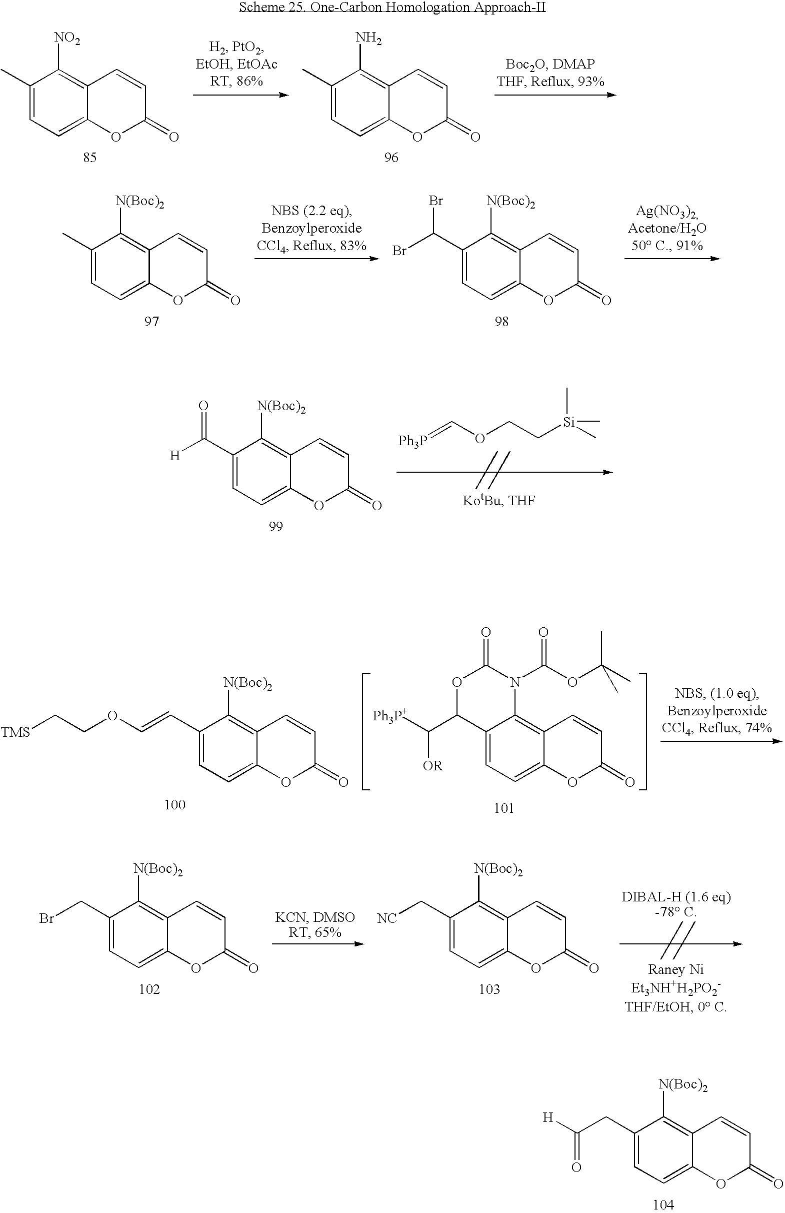

However, the Wittig reaction with methoxyphosphane did not afford product 100, which have provided the desired homologated aldehyde. Instead, compound 101 was detected as the major product. Since the mono-bromide 102 could be easily accessed from this route, it was subjected to KCN to give the compound 103. However, the direct conversion of the nitrile group to an aldehyde either DIBAL-H at 78° C. or selective catalytic hydrogenation using nickel as catalyst and Et₃NH⁺H₂PO₂⁻ as the hydrogen source was unsuccessful.

The nitrile group could be hydrolyzed to a carboxylic acid, which could then be reduced to alcohol. Since Boc groups cannot survive these hydrolysis, installation of the amino group in the later stage of synthesis might be a way to overcome this problem. The synthetic plan was then revised as outlined in Scheme 26.

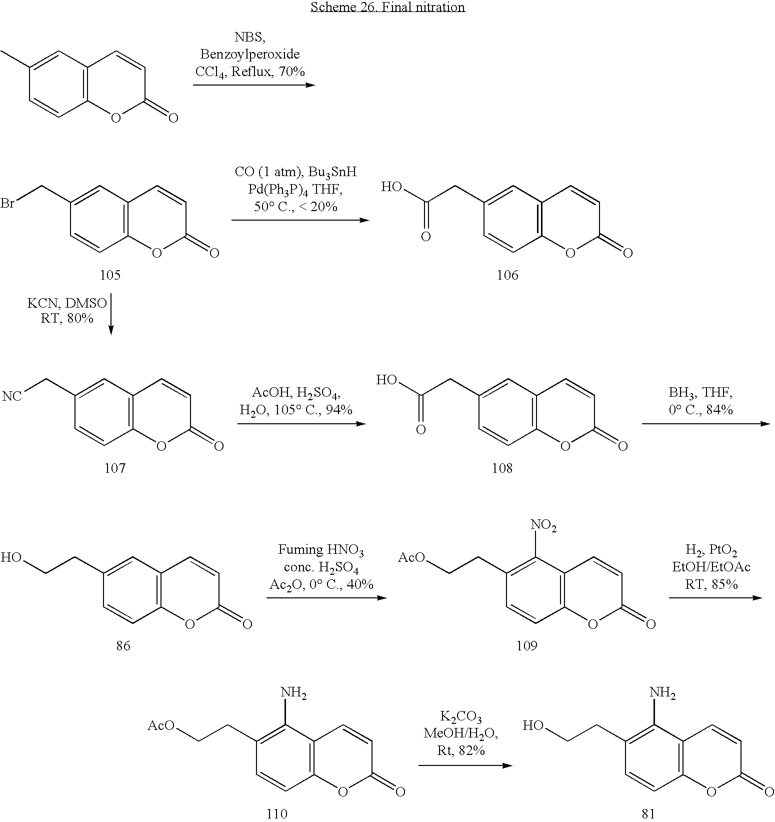

Commercially available 6-methylcoumarin was mono-brominated to give 105, which was then converted into the aldehyde 106 in one step through Pd-catalyzed formylation with carbon monoxide and tributyltinhydride. The yield was unsatisfactory, however. So, the benzylic bromide was replaced by nitrile and then hydrolyzed to carboxylic acid 108, which was then selectively reduced to give alcohol 86. The alcohol was subjected to nitration condition with fuming nitric acid in acetic anhydride at 0° C. The hydroxy group was protected as the acetate ester and nitration occurred predominantly at position 5 to give compound 109. Catalytic hydrogenation of the nitro group afforded compound 110, which was saponified to give final probe 81.

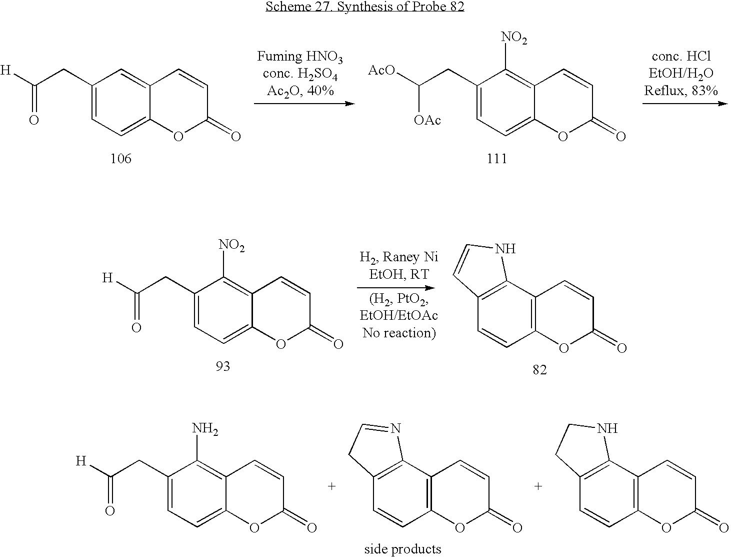

Probe 81 is very weakly fluorescent in aqueous buffer. If the corresponding pyrrolocoumarin 82 is fluorescent, the probe pair 81/82 would constitute a good fluorogenic switch. The synthesis of probe 82 is outlined in Scheme 27. Aldehyde 106 can also be made from oxidation of compound 86. When subjected to nitration conditions using fuming nitric acid in acetic anhydride, the aldehyde group was protected as the diacetate acetal and nitration predominantly occurred at the desired position 5 to give compound 111. The diacetate can be removed in refluxing conc. HCl and EtOH mixture to afford compound 93. Pyrrolocoumarin 82 was assumed to be easily accessible through catalytic hydrogenation of the nitro group and the following quick condensation and isomerization. Surprisingly, the hydrogenation of compound 93 using PtO₂ as catalyst did not proceed even over prolonged time; the starting material was thus recovered. Hydrogenation in EtOH using Raney Ni as catalyst consumed all of the nitrocoumarin 93 in 2 hours at room temperature. However, only trace amount of pyrrolocoumarin 82 was detected. A mixture of side products was formed according to ¹H-NMR and MS analyses. These results implied that the amino group at position 5 was not as nucleophilic as those at positions 6 and 7. Due to this weak nucleophilicity, the intramolecular Schiff base formation and isomerization to pyrrole did not proceed rapidly under the reduction conditions.

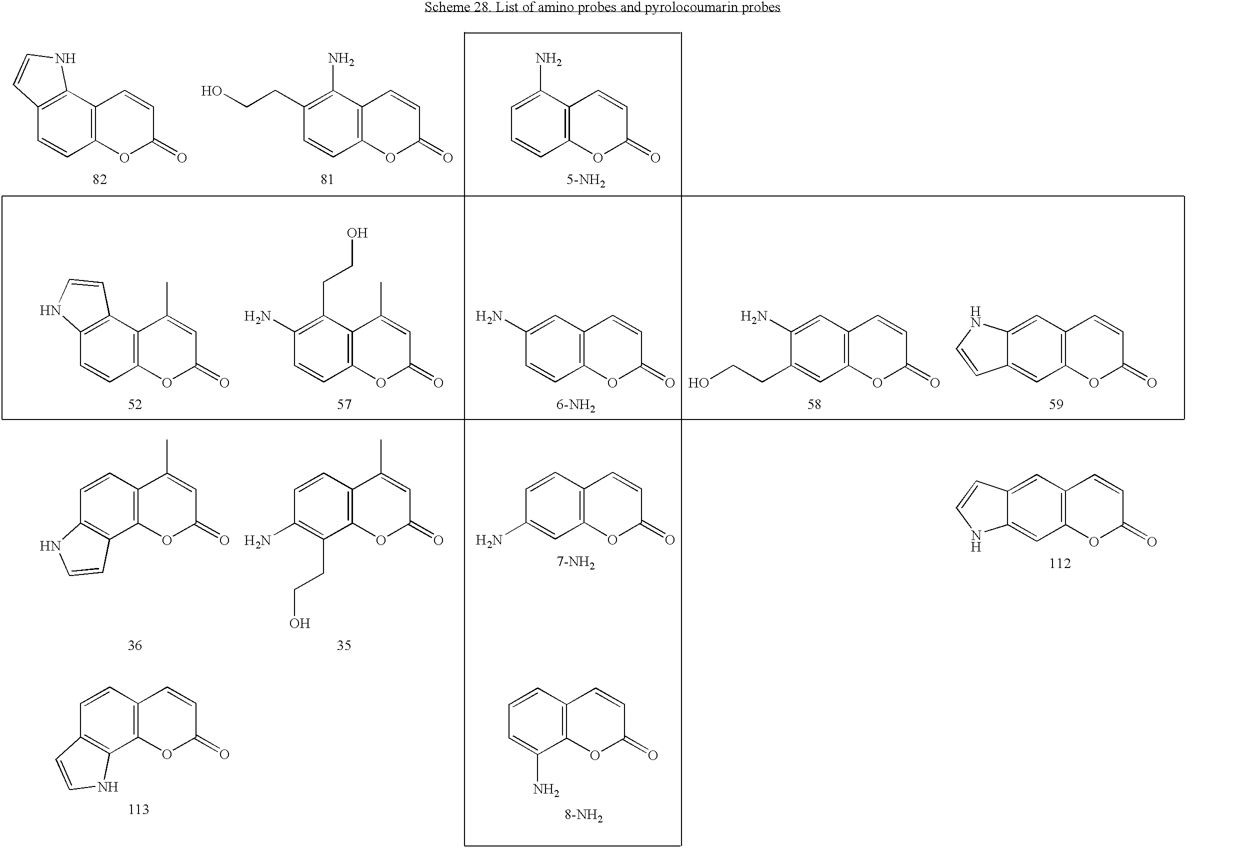

Aminocoumarin and pyrrolocoumarin probes with amino groups at positions 5, 6, and 7 were investigated by syntheses and fluorescence characterization (Scheme 28). The 7-aminocoumarins, like probe 35, are highly fluorescent, while their pyrrolocoumarin counterparts (i.e. 36) are very weakly fluorescent. Coumarin with amino group at position 5, like probe 81, possesses only weak fluorescence. However, the low reactivity of the 5-amino could not facilitate the required rapid intramolecular condensation and isomerization to form the expected pyrrolocoumarin 82, which might be quite fluorescent based on our prediction. Coumarins with amino group at position 6, like probes 57 and 58, are also very weakly fluorescent, and their corresponding pyrrolocoumarins 52 and 59 are quite fluorescent.

The probe pairs of 52/57 and 58/59 fulfilled the requirements of proper chemical transformation and distinct fluorescence readout. They were therefore further subjected to biological assays to test for the possibility of their functioning as substrates for certain dehydrogenases.

Probes for Monoamine Oxidase

Monoamine oxidase (MAO; EC 1.4.3.4) is an FAD-dependent enzyme localized in the outer membrane of mitochondria and plays an essential role in the turnover of monoamine neurotransmitters such as dopamine, serotonin and noradrenaline. Its physiological function has been widely implicated in apoptosis, immunosuppression, cytotoxicity, cell growth, and proliferation. MAO catalyzes the oxidative deamination of biogenic amines to their corresponding aldehydes, which is accompanied by the reduction of molecular oxygen to hydrogen peroxide H2O2.

MAO occurs in at least two forms, MAO-A and MAO-B, with different specificities for substrates and inhibitors. The cloning of cDNAs for MAO-A and MAO-B has demonstrated that the two isoenzyme forms are encoded by different genes, associating their different specificities for substrates and inhibitors to their corresponding primary structures. MAO A is inhibited by clorgyline and acts preferentially on serotonin and norepinephrine. It has been shown to be responsible for certain types of depression, which arise from a decrease in the concentration of brain norepinephrine and/or serotonin. MAO B is inhibited by 1-deprenyl and acts preferentially on 2-phenylethylamine and benzylamine. It is linked to Parkinson's disease as a result of its degradation of brain dopamine (Tetrud, V. W., Langston, J. W. Science. 1989, 245:519-522). Consequently, the regulation of MAO activity has been shown to be very important for the treatment of those related diseases.

MAO belongs to a family of enzymes known as flavoenzymes because of their requirement for a flavin coenzyme. The enzyme catalyzes the anaerobic conversion of amine substrates to the corresponding imines, which are released from the enzyme and hydrolyzed to the corresponding aldehydes (Scheme 29). The enzyme is inactive in the reduced flavin form and requires molecular oxygen to oxidize it back to the native form. H₂O₂ and ammonia are produced as side products from this process.

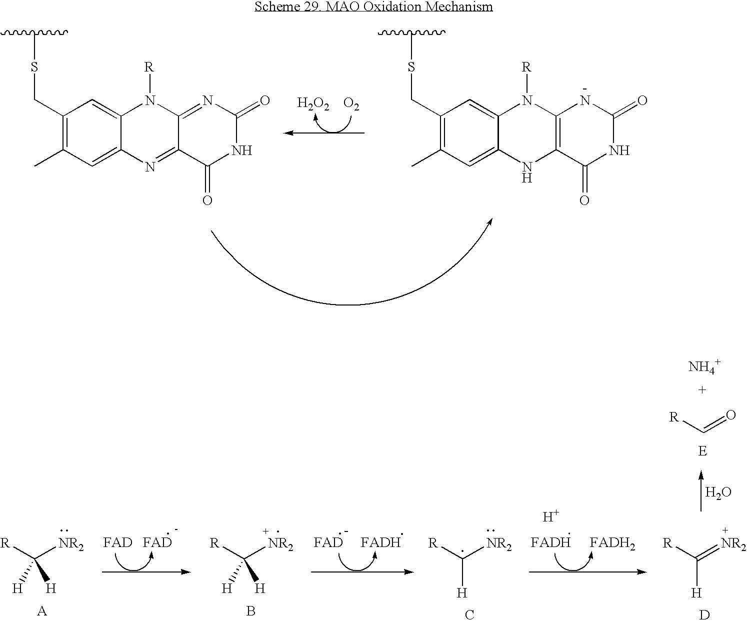

Although controversial (Walker, M. C., Edmondson, D. E. Biochemistry 1994, 33:7088) MAO catalysis is thought by many to proceed via the single electron transfer (SET) pathway illustrated in Scheme 29. According to this mechanism, one electron from the nitrogen lone pair of the substrate A is transferred to oxidized flavin (FAD) to generate the aminyl radical cation FAD⁻. Loss of a proton from B gives the radical intermediate C that undergoes a second one-electron oxidation to yield the iminium product D and reduced flavin (FADH₂).

The commonly used methods for the determination of the activity of MAO include direct spectrophotometric, fluorometric, and radiometric assays and some indirect ammonia, O₂, and H₂O₂ assays. In addition to the serious inconvenience and lack of sensitivity of discontinuous assays, like the ammonia assay, they may fail to measure the dynamic rates of enzymatic process and will never be amenable to medical imaging in vivo. Radiometric assays are limited to the labeled compounds that are commercially available and present safety hazards. Fluorometric assays involving the measurement of H₂O₂ by a coupled fluorogenic reaction are continuous, but they require the use of a second enzyme. Sensitive, direct, and continuous fluorometric assays of MAO are extremely desirable for the modern biological and clinical applications, e.g., in vivo fluorescence imaging.

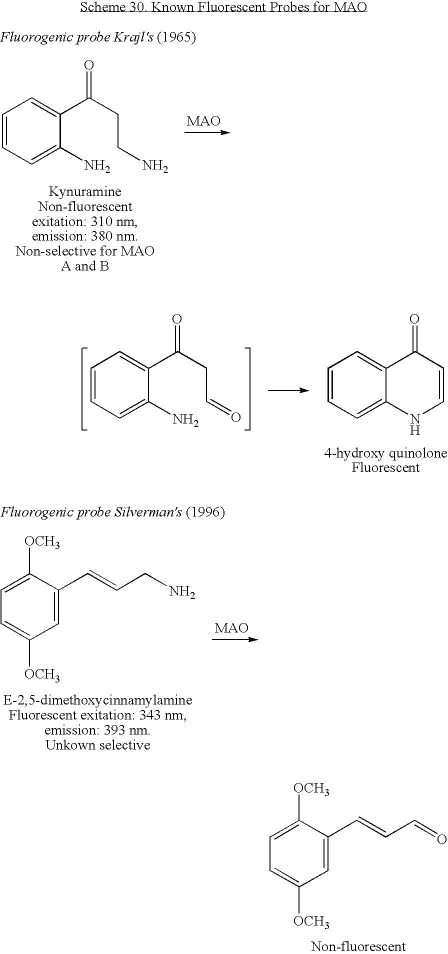

However, the development of practical fluorescent reporting substrate probes for MAO still present a huge challenge in the booming area of molecular imaging. The selective MAO-A substrate seretonin has been used to measure MAO-A activity but the aldehyde metabolite does not possess a good fluorophore. Kynuramine, an intermediate in tryptophan metabolism, was found able to be oxidized by MAO to generate an aldehyde that cyclise to form 4-hydroxyquinolone (Scheme 30). Since the discovery that kynuramine is non-fluorescent and 4-hydroxyquinolone is fluorescent, kynuramine has been used as a fluorogenic probe to estimate MAO activity in vitro. However, its lack of sensitivity and selectivity, and most importantly short emission wavelength, limits its application in in vivo fluorescence imaging of MAO. Silverman et al recently reported a synthetic fluorescent probe E-2,5-dimethoxycinnamylamine as a good substrate for MAO. However, the amine itself is fluorescent while the corresponding aldehyde is not. Although the activity of MAO can be determined by following the decrease in fluorescence at 393 nm, it will not be suitable for in vivo study.

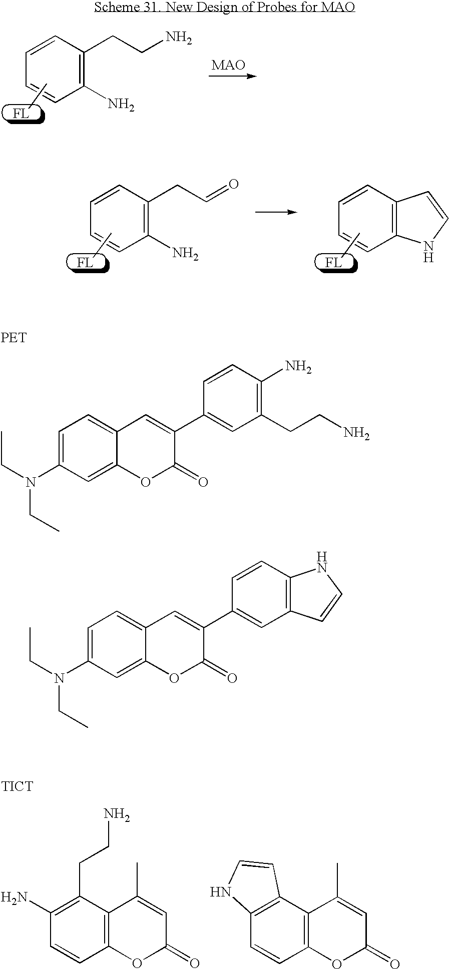

MAO is known to be a relatively promiscuous enzyme. It can catalyze the oxidation of a variety of exogenous amines including primary, secondary, and tertiary alkyl and arylalkyl amines, although the preference is for primary amines. Herein, we proposed that we could take advantage of the promiscuity of this enzyme and adapt our successful design for dehydrogenases to a new design for MAOs. The obvious adaptation is to replace the hydroxy group with a primary amino group as shown in Scheme 31 Facile syntheses of those diamine probes would allow us to quickly test whether they could function as MAOs substrates.

Synthesis of Probes for Monoamine Oxidases.

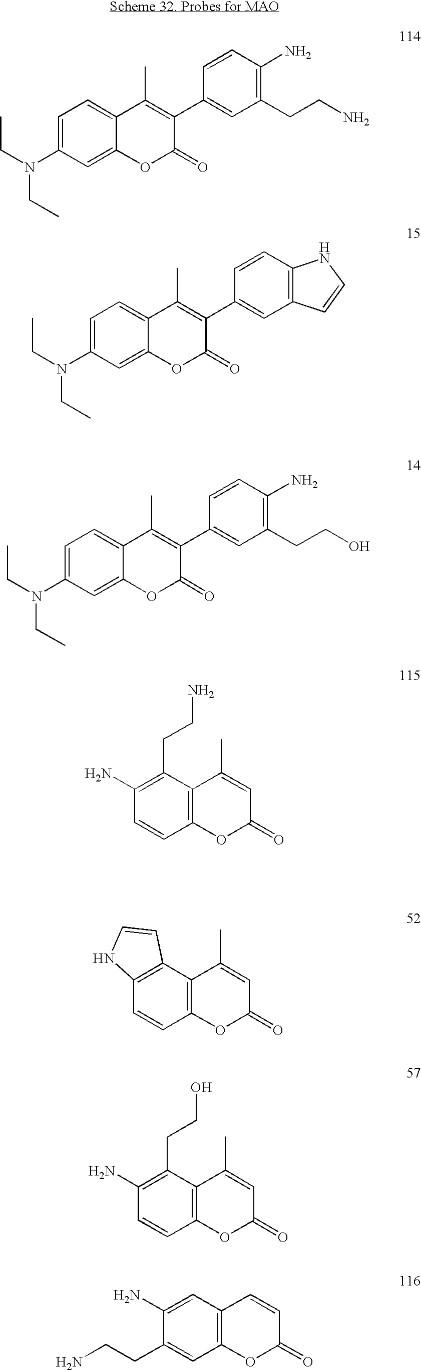

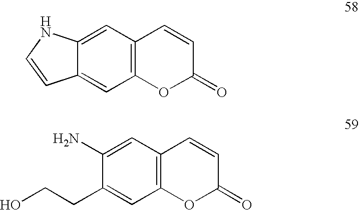

Bases on our three successful candidate probes for dehydrogenases 14, 57, and 59, three corresponding diamine probes 114, 115, and 116 were proposed as outlined in Scheme 32. We not only adapted the versatile structural motif and built-in mechanism of these dehydrogenase probes, but also took advantage of their synthetic intermediates in the syntheses of new probes for MAO. The synthetic efforts will be discussed in the following schemes.

Pd-catalyzed Suzuki coupling of two key intermediates 18 and 126 furnished probe 118 (Scheme 33). Intermediate 117 was prepared from compound 5 through catalytic hydrogenation of the nitro group, Boc protection of the amino group was followed by selective reduction of the ester using DIBAL-H. The phenyl ethyl alcohol 119 was then tosylated and substituted with an azide group. Catalytic hydrogenation of the azide group in the presence of Boc₂O resulted in the properly protected intermediate 121.

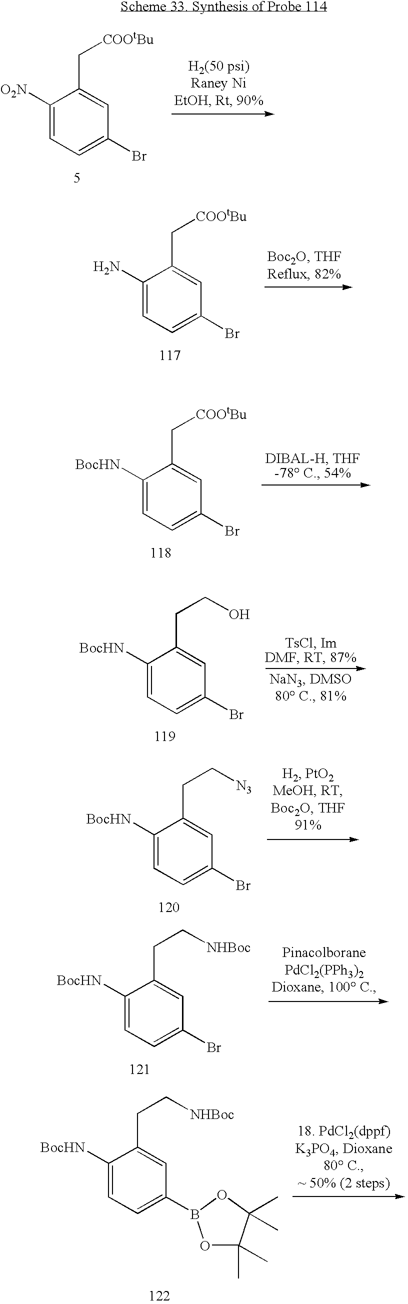

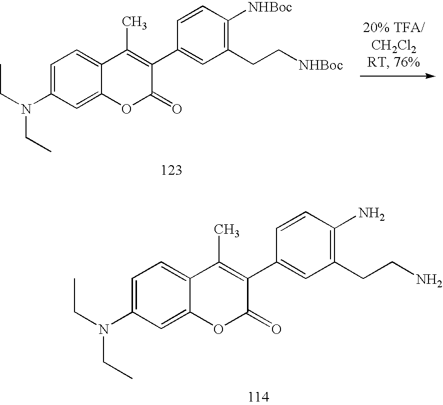

Suzuki coupling of intermediates 122 and 18 gave the product 123 with good yield. Deprotection of Boc protecting groups in 20% TFA in CH₂Cl₂ afforded the final probe 114. The diamine probe shows similar fluorescence properties as its hydroxy analogue 14. It also possesses the desired chemical stability and water solubility.

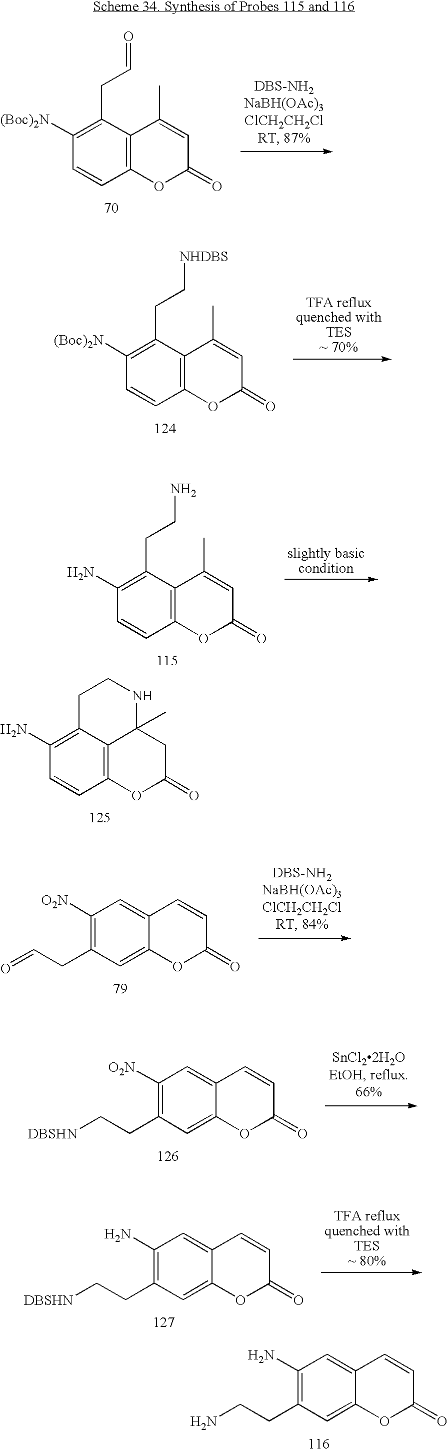

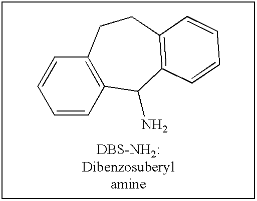

Compared to the big size and medium fluorescence signal amplification of probe 114, 115 and 116 possess more attractive structural and photo-physical features. The diamine probes can be prepared from the same intermediates as their hydroxy analogs (Scheme 34). The aldehyde group of 70 could be converted into the properly protected amino group through reductive amination; the right choice of amination reagent was critical to the synthesis. Dibenzosuberylamine (DBS—NH₂) was chosen to react with the aldehyde to afford 124 by treatment with NaBH(OAc)₃ in ClCH₂CH₂Cl. Amino protecting groups Boc and DBS can be removed simultaneously by treatment in refluxing TFA and quenching with triethylsilane. The final diamine 115 was isolated by flash chromatography and was stable when protonated, however, it was found to be very unstable once deprotonated, or even under neutral condition. The terminal ethylamino group underwent intramolecular Michael addition to the double bond of the α,β-unsaturated lactone to form 125, which is non-fluorescent. Because of the lack of stability, probe 115 was not considered for further study.

Probe 116 was prepared from aldehyde intermediate 79 using similar chemistry. Reductive amination of the aldehyde gave 126 in excellent yield. The nitro group was then reduced by SnCl₂.2H₂O in refluxing EtOH. The DBS group was removed by TFA and triethylsilane to afford the final probe 116. This diamine compound was perfectly stable and very weakly fluorescent in buffer. Since its pyrrolocoumarin 58 was fluorescent, the probe 116 was a promising candidate for further study as a potential fluorogenic probe for MAOs.

Probes 114 and 116, which both possessed the required chemical transformation and fluorescent switching ability, were then subjected to enzymatic assays with MAO A and B.

Materials and General Methods

Reagents were obtained from commercial suppliers and were used without purification. Solvents were dried when necessary. Nuclear Magnetic Resonance (NMR) spectra were recorded on Bruker 300 or 400 Fourier Transform (FT) NMR spectrometers. Spectra were taken in methanol-d₄ at 300K with the proton or carbon (3.30, 49.0) as the reference; in chloroform-d at 300K with the proton or carbon (7.26, 77.0) as the reference or in DMSO-d₆ at 300K with the proton or carbon (2.49, 39.5) as the reference. Infrared spectra were recorded on a Perkin-Elmer 1600 FTIR spectrometer. High Resolution Mass Spectra (HRMS) were obtained on a JOEL JMS-HX110 HF mass spectrometer. Spectra of MS (APCI⁺) were obtained on a JOEL JMS-LCmate LCMS mass spectrometer. UV spectra were recorded on a Perkin Elmer Lamba 19 spectrometer. Flash chromatography was performed on SILICYCLE silica gel (230-400 mesh).

Compound 3

Compound 13 (58 mg, 0.15 mmol) dissolved in 20% (v/v) TFA/CH₂Cl₂ (5 mL) was stirred at RT for 30 min. Reaction mixture was then concentrated in vacuo. The residue was purified by silica gel column chromatography with 3% to 5% MeOH in CH₂Cl₂ to give the title compound (33 mg, 76%). ¹H NMR (300 MHz; DMSO-d₆): δ, 2.41 (s, 3H), 2.68 (t, J=6.3 Hz, 2H), 3.65 (q, J=5.2 Hz, 2H), 4.65 (t, J=5.9 Hz, 1H), 5.25 (s, 2H), 6.29 (s, 1H), 6.71 (d, J=8.1 Hz, 1H), 7.39 (m, 2H), 7.56 (m, 2H), 7.71 (d, J=8.3 Hz, 1H). ¹³C NMR (100 MHz, DMSO-d₆): δ, 18.0, 34.5, 60.5, 112.0, 112.9, 114.9, 121.3, 123.5, 125.4, 125.5, 125.6, 128.7, 144.5, 147.7, 153.1, 153.7, 160.1. IR υₘₐₓ(NaCl)/cm⁻¹: 3370, 1697, 1573, 1391, 1051, 856. HRMS (FAB+) calculated for C₁₈H₁₈NO₃ m/z 296.1287, observed m/z 296.1290.

Compound 4

Compound 15 (68 mg, 0.17 mmol) dissolved in 20% (v/v) TFA/CH₂Cl₂ (5 mL) was stirred at room temperature for 30 min. Reaction mixture was then concentrated in vacuo. The residue was purified by silica gel column chromatography with 2:1 hex/EtOAc to give the title compound (33 mg, 70%). ¹H NMR (300 MHz, DMSO-d₆): δ, 2.42 (s, 3H), 6.33 (s, 1H), 6.52 (s, 1H), 7.41 (s, 1H), 7.50 (s, 2H), 7.67 (s, 1H), 7.75 (d, J=8.4 Hz, 1H), 7.96 (d, J=8.2 Hz, 1H), 11.2 (s, 1H). ¹³C NMR (100 MHz, DMSO-d₆): δ, 18.0, 101.8, 112.0, 113.4, 113.5, 117.6, 118.8, 120.4, 122.6, 125.6, 126.4, 128.3, 129.2, 136.1, 145.6, 153.0, 153.5, 160.0. IR υₘₐₓ(NaCl)/cm⁻¹: 3268, 1693, 1606, 1388, 1154, 891. HRMS (FAB+) calculated for Cl₈H₁₄NO₂ m/z 276.1025, observed m/z 276.1008.

Compound 5

To a suspension of potassium tert-butoxide (7.08 g, 63 mmol) in anhydrous DMF (100 mL) with stirring at 0° C. was added dropwise a solution of 1-bromo-4-nitrobenzene (5.05 g, 25 mmol) and tert-butyl chloroacetate (3.92 mL, 27.5 mmol) in DMF (50 mL), and the mixture was stirred at 0° C. for 3 h. The resulting mixture was acidified with aqueous 5% KHSO₄ and extracted with EtOAc. The organic layer was washed successively with water and brine, dried over magnesium sulfate, and concentrated in vacuo. The residue was purified by silica gel column chromatography with 20:1 hex/EtOAc to give the title compound (4.34 g, 55%). ¹H NMR (300 MHz, CDCl₃): δ, 1.44 (s, 9H), 3.91 (s, 2H), 7.50 (d, J=2.2 Hz, 1H), 7.59 (dd, J=2.2, 8.7 Hz, 1H), 7.98 (d, J=8.7 Hz, 1H). ¹³C NMR (75 MHz, CDCl₃): δ, 27.9, 40.8, 80.2, 126.6, 128.2, 131.5, 132.4, 136.1, 147.6, 168.4. IR υₘₐₓ(NaCl)/cm⁻¹: 2980, 1732, 1524, 1345, 1151. MS (APCI+) calculated for C₁₂H₁₄BrNO₄ m/z 316.15, observed m/z 316.8.

Compound 6

To a solution of 5 (126 mg, 0.4 mmol) in anhydrous THF (5 mL) under Ar at 0° C. was added dropwise DIBAL-H (1.2 mL, 1.2 mmol, 1.0 M in hexane), and the mixture was stirred at 0° C. for 1 h. 0.5 N HCl (10 mL) and EtOAc (20 mL) was added and the organic layer was washed with brine, dried over magnesium sulfate, and concentrated in vacuo. The residue was purified by silica gel column chromatography with 4:1 hex/EtOAc to give the title compound (83 mg, 84%). ¹H NMR (400 MHz, CDCl₃): δ, 2.22 (s, 1H), 3.11 (t, J=6.3 Hz, 2H), 3.90 (t, J=6.3 Hz, 2H), 7.49 (dd, J=1.9, 8.7 Hz, 1H), 7.58 (d, J=1.9 Hz, 1H), 7.79 (d, J=8.7 Hz, 1H). ¹³C NMR (100 MHz, CDCl₃): δ, 35.9, 62.2, 126.1, 127.5, 130.5, 135.4, 135.8, 148.1. IR υₘₐₓ(NaCl)/cm⁻¹: 3367, 1522, 1345, 1043. MS (APCI+) calculated for C₈H₈BrNO₃ m/z 246.06, observed m/z 246.7.

Compound 7

TBDMSCl (0.53 g, 3.54 mmol) was added slowly to a solution of compound 6 (0.83 g, 3.37 mmol) and imidazole (0.48 g, 7.08 mmol) in dry DMF (20 mL) with stirring at 0° C. After stirring at room temperature overnight, water (100 mL) and EtOAc (100 mL) was added, and the organic layer was separated, washed with water and brine, dried over MgSO₄, and concentrated in vacuo. The residue was purified by silica gel column chromatography with 30:1 Hex/EtOAc to give the title compound (1.06 g, 87%). ¹H NMR (400 MHz, CDCl₃): δ, −0.06 (s, 6H), 0.87 (s, 9H), 3.09 (t, J=6.0 Hz, 2H), 3.89 (t, J=6.0 Hz, 2H), 7.49 (dd, J=2.1, 8.7 Hz, 1H), 7.59 (d, J=2.1 Hz, 1H), 7.79 (d, J=8.7 Hz, 1H). ¹³C NMR (100 MHz, CDCl₃): δ, −5.4, 18.3, 25.9, 36.1, 62.5, 125.8, 126.9, 130.1, 136.1, 136.3, 148.1. IR υₘₐₓ(NaCl)/cm⁻¹: 1524, 1350, 1255, 1097. MS (APCI+) calculated for C₁₄H₂₂BrNO₃Si m/z 360.32, observed m/z 362.1.

Compound 8

Compound 7 (1.03 g, 2.86 mmol) and Zn(OAc)₂.H₂O (626 mg, 2.86 mmol) in MeOH (20 mL) was hydrogenated over PtO₂ at room temperature under an atmospheric pressure of hydrogen for 4 h. The reaction mixture was concentrated in vacuo and then dissolved in 100 mL of EtOAc and 50 mL of water. The organic layer was washed with brine and dried over magnesium sulfate and concentrated in vacuo. The residue was purified by silica gel column chromatography with 20:1 Hex/EtOAc to give the title compound (0.86 g, 91%). ¹H NMR (400 MHz, CDCl₃): δ, −0.02 (s, 6H), 0.85 (s, 9H), 2.74 (t, J=5.9 Hz, 2H), 3.85 (t, J=5.9 Hz, 2H), 4.30 (bs, 1H), 6.64 (d, J=9.1 Hz, 1H), 7.14(m, 2H). ¹³C NMR (100 MHz, CDCl₃): δ, −5.3, 18.4, 26.0, 35.0, 64.5, 111.1, 117.8, 128.2, 129.8, 132.8, 143.3. IR υₘₐₓ(NaCl)/cm⁻¹: 3353, 1625, 1490, 1256, 1095, 834. MS (APCI+) calculated for C₁₄H₂₄BrNOSi m/z 330.34, observed m/z 332.1.

Compound 9

Di-tert-butyl dicarbonate (70 mg, 0.323 mmol) was added to a solution of compound 8 (97 mg, 0.294 mmol) in anhydrous THF (5 mL), and the mixture was refluxed under Ar for 20 h. After the solvent was removed in vacuo, the residue was purified by silica gel column chromatography with 50:1 Hex/EtOAc to give the title compound (103 mg, 82%). ¹H NMR (400 MHz, CDCl₃) δ, 0.00 (s, 6H), 0.86 (s, 9H), 1.50 (s, 9H), 2.77 (t, J=4.5 Hz, 2H), 3.86 (t, J=4.5 Hz, 2H), 7.21 (s, 1H), 7.31 (d, J=8.7 Hz, 1H), 7.70 (d, J=8.7 Hz, 1H), 8.07 (bs, 1H). ¹³C NMR (100 MHz, CDCl₃): δ, −5.7, 18.4, 25.9, 28.4, 35.2, 65.7, 80.0, 115.9, 123.5, 129.9, 132.7, 133.1, 136.9, 153.3. IR υₘₐₓ(NaCl)/cm⁻¹: 3343, 1730, 1516, 1237, 1160. HRMS (FAB⁺) calculated for C₁₉H₃₂BrNO₃Si m/z 429.1335, observed m/z 429.1321.

Compound 10

A flask charged with PdCl₂(PPh₃)₂ (10 mg, 0.015 mmol) and compound 9 (215 mg, 0.5 mmol) was flushed with Ar. Anhydrous dioxane (5 mL), freshly-distilled triethylamine (208 μL, 1.5 mmol) and pinacolborane (109 μL, 0.75 mmol) were then added. After being stirred at 100° C. for 4 h, the product was extracted with EtOAc, washed with water (3×10 mL) and brine, dried over anhydrous MgSO₄, concentrated in vacuo and used in the following reaction without further purification.

Compound 11

N-phenyltrifluoromethanesulfonimide (3.02 g, 8.51 mmol) and triethylamine (1.63 mL, 11.74 mmol) was added to a solution of 7-hydroxy-4-methylcoumarin (1.03 g, 5.87 mmol) in CH₂Cl₂ (ACS grade, 50 mL). The reaction mixture was stirred at RT for 2 h. After the solvent was removed in vacuo, the residue was purified by silica gel column chromatography with 3:1 Hex/EtOAc to give the title compound (1.66 g, 92%). ¹H NMR (400 MHz, CDCl₃): δ, 2.48 (d, J=1.2 Hz, 3H), 6.36 (d, J=1.2 Hz, 1H), 7.26 (m, 2H), 7.72 (d, J=8.6 Hz, 1H). ¹³C NMR (100 MHz, CDCl₃): δ, 18.8, 110.3, 115.7, 117.2, 126.2, 150.5, 151.0, 153.8, 159.1. IR υₘₐₓ(NaCl)/cm⁻¹: 1738, 1628, 1434, 1212, 1127, 978. MS (APCI+) calculated for C₂₂H₂₇N₂O₃ m/z 367.2022, observed m/z 367.2026.

Compound 12

A flask containing crude compound 10 was flushed with Ar. PdCl₂(dppf) (37 mg, 0.05 mmol), compound 11 (139 mg, 0.45 mmol), K₃PO₄ (424 mg, 2 mmol) and dioxane (5 mL) were then added. The mixture was then stirred at 80° C. for 20 h. The product was then extracted with EtOAc, washed with water (3×30 mL) and brine, dried over anhydrous MgSO₄, concentrated in vacuo. The residue was purified by silica gel column chromatography with 6:1 hex/EtOAc to give the title compound (˜60%, over 2 steps). ¹H NMR (300 MHz, CDCl₃): δ, 0.03 (s, 6H), 0.88 (s, 9H), 1.53 (s, 9H), 2.45 (d, J=1.1 Hz, 3H), 2.90 (t, J=4.6 Hz, 2H), 3.94 (t, J=4.6 Hz, 2H), 6.26 (d, J=1.1 Hz, 1H), 7.37 (m, 1H), 7.51 (m, 3H), 7.62 (d, J=8.7 Hz, 1H), 7.94 (d, J=8.4 Hz, 1H), 8.23 (s, 1H). MS (APCI+) calculated for C₂₉H₃₉NO₅Si m/z 509.71, observed m/z 510.2.

Compound 13

Compound 12 (130 mg, 0.26 mmol) was dissolved in anhydrous THF (5 mL). TBAF (1 M in THF, 0.33 ml, 0.33 mmol) was added dropwise under Ar at RT. The reaction mixture was stirred at room temperature for 30 min. 20 mL of EtOAc and 20 mL of water were added. The organic layer was washed with brine, dried over MgSO₄ and concentrated in vacuo. The residue was purified by silica gel column chromatography with 2:1 Hex/EtOAc to give the title compound (74 mg, 74%). ¹H NMR (400 MHz, CDCl₃): δ, 1.52 (s, 9H), 2.36 (bs, 1H), 2.43 (s, 3H), 2.90 (t, J=5.5 Hz, 2H), 3.97 (t, J=5.6 Hz, 2H), 6.23 (s, 1H), 7.38 (s, 1H), 7.45 (m, 3H), 7.59 (d, J=8.2 Hz, 1H), 7.86 (d, J=8.1 Hz, 1H), 7.98 (s, 1H). ¹³C NMR (100 MHz, CDCl₃): δ, 18.8, 28.5, 35.0, 64.2, 80.3, 114.2, 114.4, 118.4, 122.5, 122.7, 124.7, 125.7, 128.8, 131.1, 134.0, 137.7, 144.1, 152.1, 153.3, 153.5, 160.8. IR υₘₐₓ(NaCl)/cm⁻¹: 3325, 1716, 1615, 1526, 1390, 1241, 1158, 1051. MS (APCI⁺) calculated for C₂₃H₂₅NO₅ m/z 395.45, observed m/z 396.2.

Compound 14 and 15

Compound 13 (20 mg, 0.056 mmol) was dissolved in CH₂Cl₂ (5 mL). Dess Martin periodinane (0.18 ml, 0.084 mmol, 15% in CH₂Cl₂,) was added dropwise at RT. The reaction mixture was stirred at room temperature for 30 min. 20 mL of EtOAc and 20 mL of water was added. The organic layer was washed with brine, dried over MgSO₄ and concentrated in vacuo. The residue was purified by silica gel column chromatography with 2:1 Hex/EtOAc to give compound 14 (13 mg, 60%) and compound 15 (6 mg, 30%). 14. ¹H NMR (400 MHz, CDCl₃): δ, 1.63 (s, 9H), 2.45 (s, 3H), 3.31 (d, J=17.2 Hz, 1H), 3.42 (dd, J=7.6, 17.3 Hz, 1H), 6.07 (bs, 1H), 6.27 (s, 1H), 7.46 (m, 5H), 7.65 (d, J=12.6 Hz, 1H). IR υₘₐₓ(NaCl)/cm⁻¹: 3425, 1723, 1620, 1375, 1262. MS (FAB⁺) calculated for C₂₃H₂₃NO₅ m/z 393.43, observed m/z 394.3. 15. ¹H NMR (400 MHz, CDCl₃): δ, 1.69 (s, 9H), 2.45 (d, J=1.2 Hz, 3H), 6.26 (d, J=1.2 Hz, 1H), 6.62 (d, J=3.7 Hz, 1H), 7.53-7.77 (m, 5H), 7.79 (d, J=1.3 Hz, 1H), 8.21 (d, J=8.4 Hz, 1H). ¹³C NMR (100 MHz, CDCl₃): δ, 18.8, 28.3, 84.0, 107.3, 114.3, 114.9, 115.5, 118.3, 119.4, 123.0, 123.3, 124.6, 126.7, 131.0, 133.4, 135.0, 145.1, 149.3, 152.0, 153.0, 160.7. IR υₘₐₓ(NaCl)/cm⁻¹: 1732, 1367, 1248, 1162. HRMS (FAB⁺) calculated for C₁₈H₁₄NO₂⁻ m/z 376.1549, observed m/z 376.1558.

Compound 16

Prepared through Boc deprotection with TFA/CH₂Cl₂ in a same manner as compound 3. ¹H NMR (300 MHz, CDCl₃): δ, 1.21 (t, J=7.1 Hz, 6H), 2.24 (s, 3H), 2.80 (t, J=6.0 Hz, 2H), 3.30 (bs, 1H), 3.42 (q, J=7.1 Hz, 4H), 3.90 (t, J=6.0 Hz, 2H), 6.54 (d, J=2.5 Hz, 1H), 6.61 (dd, J=2.6, 9.0 Hz, 1H), 6.76 (d, J=8.6 Hz, 1H), 6.98 (s, 1H), 6.99 (d, J=8.4 Hz, 1H), 7.43 (d, J=9.0 Hz, 1H). ¹³C NMR (75 MHz, CDCl₃): δ, 12.5, 16.4, 34.8, 44.7, 63.2, 97.5, 108.5, 109.8, 116.1, 121.1, 124.1, 126.0, 129.8, 132.6, 144.3, 147.9, 150.0, 154.9, 162.6. IR υₘₐₓ(NaCl)/cm⁻¹: 3365, 1692, 1616, 1523, 1273, 1077, 730. HRMS (FAB⁺) calculated for C₂₂H₂₇N₂O₃ m/z 367.2022, observed m/z 367.2026.

Compound 17

A flask charged with PdCl₂(dppf)₂ (11 mg, 0.015 mmol) and compound 18 (80 mg, 0.5 mmol) was flushed with Ar. DMF (5 mL), H₂O (0.5 mL) and K₂CO₃ (138 mg, 1.0 mmol) were then added. After being stirred at 90° C. for 3 h, the product was extracted with EtOAc, washed with water (3×10 mL) and brine, dried over anhydrous MgSO₄, concentrated in vacuo. The residue was purified by silica gel flash chromatography with 4:1 Hex/EtOAc to give the title compound (149 mg, 86%). ¹H NMR (400 MHz, DMSO-d₆): δ, 1.22 (t, J=7.1 Hz, 6H), 2.25 (s, 3H), 3.43 (t, J=7.1 Hz, 4H), 6.53 (m, 1H), 6.58 (d, J=2.4 Hz, 1H), 6.63 (dd, J=2.6, 9.0 Hz, 1H), 7.09 (dd, J=1.5, 8.4 Hz, 1H), 7.18 (t, J=2.7 Hz, 1H), 7.37 (d, J=8.4 Hz, 1H), 7.47 (d, J=9.0 Hz, 1H), 7.53 (s, 1H), 8.38 (s, 1H). ¹³C NMR (100 MHz, DMSO-d₆): δ, 12.4, 16.4, 44.7, 97.6, 102.6, 108.5, 109.9, 111.0, 122.3, 122.5, 124.3, 124.6, 126.0, 126.5, 127.8, 135.3, 148.2, 149.9, 155.0, 162.8. IR υₘₐₓ(NaCl)/cm⁻¹: 3337, 1688, 1616, 1522, 1410, 1077, 731. HRMS (FAB⁺) calculated for C₂₂H₂₃N₂O₃ m/z 347.1760, observed m/z 347.1764.

Compound 18

Known compound, prepared according to literature (Gordeeva, N. A., Kirpichenok, M. A., Patalakha, N. S., Grandberg, I. I. Khimiya Geterotsiklicheskikh Soedinenii 1990, 12:1600-9).

Compound 19

Prepared from compound 20 in a same manner as compound 10.

Compound 20

Known compound, prepared according to literature (Torraca, K. E., Huang, X., Parrish, C. A., Buchwald, S. L. J. Amer. Chem. Soc. 2001, 123:10770-10771).

Compound 22

Prepared from compound 18 and 10 in a same manner as compound 12. ¹H NMR (300 MHz, CDCl₃): δ, 0.03 (s, 6H), 0.88 (s, 9H), 1.23 (t, J=7.1 Hz, 6H), 1.52 (s, 9H), 2.21 (s, 3H), 2.84 (t, J=5.3 Hz, 2H), 3.42 (q, J=7.1 Hz, 4H), 3.90 (t, J=5.3 Hz, 2H), 6.54 (d, J=2.3 Hz, 1H), 6.62 (d, J=7.3 Hz, 1H), 7.06 (d, J=1.9 Hz, 1H), 7.13 (d, J=8.3 Hz, 1H), 7.43 (d, J=9.0 Hz, 1H), 7.85 (d, J=8.4 Hz, 1H), 8.14 (s, 1H). ¹³C NMR (75 MHz, CDCl₃): δ, −5.6, 12.4, 16.6, 18.4, 26.3, 28.4, 35.5, 44.8, 65.9, 79.8, 97.6, 108.6, 109.8, 121.0, 121.6, 126.0, 129.3, 130.3, 130.6, 132.2, 137.0, 148.2, 150.0, 153.5, 155.0, 162.0. IR υₘₐₓ(NaCl)/cm⁻¹: 3339, 2930, 1716, 1618, 1522, 1161, 1079, 829. HRMS (FAB⁺) calculated for C₃₃H₄₈N₂O₅Si m/z 580.3333, observed m/z 580.3316.

Compound 23

Prepared through TBAF and Boc deprotection of compound 27 in a same manner as compound 16. ¹H NMR (400 MHz, CDCl₃): δ, 1.98 (t, J=6.4 Hz, 4H), 2.20 (s, 3H), 2.78 (q, J=6.3 Hz, 4H), 2.92 (t, J=6.5 Hz, 2H), 3.2 (bs, 1H), 3.24 (m, 4H), 3.86 (t, J=6.2 Hz, 2H), 6.72 (d, J=8.6 Hz, 1H), 6.95 (m, 2H), 7.03 (s, 1H). ¹³C NMR (100 MHz, CDCl₃): δ, 16.4, 20.3, 20.6, 21.5, 27.8, 34.0, 49.5, 49.9, 106.3, 109.2, 118.3, 118.6, 122.5, 123.6, 130.2, 130.6, 133.4, 134.5, 136.0, 145.9, 150.1, 150.4. IR υₘₐₓ, (NaCl)/cm⁻¹: 3363, 2931, 1684, 1614, 1557, 1507, 1310, 1181. HRMS (FAB⁺) calculated for C₂₄H₂₇N₂O₃ m/z 391.2022, observed m/z 391.2007.

Compound 24

Prepared from compound 26 in a same manner as compound 17. ¹H NMR (400 MHz, DMSO-d₆): δ, 1.90 (m, 4H), 2.14 (s, 3H), 2.76 (m, 4H), 3.24 (m, 4H), 6.42 (s, 1H), 6.92 (dd, J=1.4, 8.3 Hz, 1H), 7.15 (s, 1H), 7.35 (m, 2H), 7.40 (d, J=8.3 Hz, 1H), 11.1 (s, 1H). ¹³C NMR (100 MHz, DMSO-d₆): δ, 16.3, 20.0, 20.3, 21.1, 27.1, 48.6, 49.1, 100.9, 105.1, 108.4, 110.5, 117.4, 120.4, 121.6, 122.2, 123.3, 125.3, 125.6, 127.1, 134.7, 144.6, 147.6, 149.0 160.8. IR υₘₐₓ(NaCl)/cm⁻¹: 3363, 2928, 1685, 1614, 1557, 1313, 1169, 731. HRMS (FAB⁺) calculated for C₂₄H₂₃N₂O₂ m/z 371.1760, observed m/z 371.1742.

Compound 25

Prepared in a same manner as compound 17. ¹H NMR (400 MHz, CDCl₃): δ, 2.00 (m, 4H), 2.81 (t, J=6.2 Hz, 2H), 2.93 (t, J=6.5 Hz, 2H), 3.30 (m, 4H), 6.56 (s, 1H), 7.09 (dd, J=1.6, 8.4 Hz, 1H), 7.22 (m, 2H), 7.42 (d, J=8.3 Hz, 1H), 7.51 (s, 1H), 8.20 (s, 1H). MS (APCI+) calculated for C₂₄H₁₉F₃N₂O₂ m/z 424.42, observed m/z 424.9.

Compound 26

Known compound, prepared according to literature (Schiedel, M. S., Briehn, C. A., Bauerle, P. Angew. Chem. Int. Ed. Engl. 2001, 40:4677-4680).

Compound 27

Prepared from compound 26 and 10 in a same manner as compound 12. ¹H NMR (400,MHz, CDCl₃): δ, 0.02 (s, 6H), 0.88 (s, 9H), 1.23 (t, J=7.1 Hz, 6H), 1.52 (s, 9H), 1.98 (m, 4H), 2.17 (s, 3H), 2.79 (t, J=6.4 Hz, 2H), 2.84 (t, J=5.3 Hz, 2H), 2.92 (t, J=6.5 Hz, 2H), 3.25 (m, 4H), 3.90 (t, J=5.2 Hz, 2H), 7.03 (m, 2H), 7.10 (dd, J=2.0, 8.3 Hz, 1H), 7.84 (d, J=8.2 Hz, 1H), 8.1 (s, 1H). ¹³C NMR (100 MHz, CDCl₃): δ, −5.4, 16.6, 18.7, 20.6, 21.0, 21.9, 26.2, 27.9, 28.6, 35.6, 49.6, 50.0, 65.9, 79.7, 106.5, 109.4, 117.8, 120.3, 121.4, 122.0, 129.2, 130.3, 130.5, 132.1, 136.7, 145.0, 148.0, 149.8, 153.2, 161.9. IR υₘₐₓ(NaCl)/cm⁻¹: 2943, 1701, 1425, 1378, 1309, 1184. HRMS (FAB⁺) calculated for C₃₅H₄₈N₂O₅Si m/z 604.85, observed m/z 605.6.

Compound 28

Compound 32 (54 mg, 0.12 mmol) (dry solid in flask) was heated at 180° C. for 30 min under Ar. The product was purified by silica gel column chromatography with 1:1 Hex/EtOAc to give title compound (42 mg, 78%) ¹H NMR (400 MHz, CDCl₃): δ, 1.22 (t, J=7.1 Hz, 6H), 2.24 (s, 3H), 3.43 (q, J=7.1 Hz, 4H), 6.52 (s, 1H), 6.59 (s, 1H), 6.65 (d, J=6.5 Hz, 1H), 6.99 (d, J=8.1 Hz, 1H), 7.17 (t, J=2.8 Hz, 1H), 7.26 (s, 1H), 7.32 (s, 1H), 7.47 (d, J=9.0 Hz, 1H), 7.65 (d, J=8.1 Hz, 1H), 8.40 (s, 1H). ¹³C NMR (100 MHz, CDCl₃): δ, 12.4, 16.4, 44.8, 97.5, 102.3, 108.5, 109.8, 113.1, 120.3, 122.2, 124.8, 126.0, 127.3, 128.7, 135.8, 148.4, 150.0, 155.0 162.7. IR υₘₐₓ(NaCl)/cm⁻¹: 2975, 1689, 1614, 1587, 1343, 1145, 818. MS (APCI⁺) calculated for C₂₂H₂₂N₂O₂ m/z 346.42, observed m/z 347.2.

Compound 30

Known compound, prepared according to literature (Torraca, K. E., Huang, X., Parrish, C. A., Buchwald, S. L. J. Amer. Chem. Soc. 2001, 123:10770-10771.)

Compound 31

Prepared from compound 30 in a same manner as compound 19.

Compound 32

Prepared from compound 18 and 31 in a same manner as compound 12. ¹H NMR (400 MHz, CDCl₃): δ, 1.22 (t, J=7.0 Hz, 6H), 1.64 (s, 9H), 2.26 (s, 3H), 3.42 (q, J=7.1 Hz, 4H), 6.57 (m, 2H), 6.63 (m, 1H), 7.16 (dd, J=1.4, 8.0 Hz, 1H), 7.46 (d, J=9.0 Hz, 1H), 7.59 (m, 2H), 8.12 (s, 1H). ¹³C NMR (100 MHz, CDCl₃): δ, 12.7, 16.6, 44.8, 83.5, 97.4, 107.0, 108.4, 109.5, 117.1, 120.5, 121.6, 124.9, 125.8, 126.1, 129.8, 131.1, 134.9, 148.0, 149.3, 149.8, 154.8, 161.9. IR υₘₐₓ(NaCl)/cm⁻¹: 2974, 1708, 1617, 1589, 1342, 1157, 1126. MS (APCI⁺) calculated for C₂₇H₃₀N₂O₄ m/z 446.54, observed m/z 447.3.

Compound 33

¹H NMR (400 MHz, CD₃OD): δ, 3.02 (t, J=6.6 Hz, 2H), 3.75 (t, J=6.6 Hz, 2H), 6.38 (d, J=9.8 Hz, 1H), 7.04 (s, 2H), 8.19 (d, J=9.8 Hz, 1H). ¹³C NMR (100 MHz, CD₃OD): δ, 30.3, 62.3, 115.8, 116.0, 119.4, 121.0, 121.8, 143.1, 144.1, 148.7, 163.1. IR υₘₐₓ(NaCl)/cm⁻¹: 3356, 1692, 1617, 1527, 1273. HRMS (FAB⁺) calculated for C₁₁H₁₂NO₃ m/z 205.0739, observed m/z 205.0744.

Compound 34

¹H NMR (400 MHz, DMSO-d₆): δ, 6.44 (d, J=9.4 Hz, 1H), 6.91 (s, 1H), 7.11 (d, J=8.7 Hz, 1H), 7.55 (d, J=2.2 Hz, 1H), 7.64 (d, J=8.7 Hz, 1H), 8.44 (d, J=9.5 Hz, 1H), 11.6 (bs, 1H). ¹³C NMR (100 MHz, DMSO-d₆): δ, 99.5, 109.6, 109.8, 113.8, 115.6, 123.8, 127.5, 131.7, 141.7, 148.7, 160.4. IR υₘₐₓ(NaCl)/cm⁻¹: 3231, 1672, 1595, 1363, 1171, 920. HRMS (FAB⁺) calculated for C₁₁H₈NO₂ m/z 186.0550, observed m/z 186.0560.

Compound 36

Known compound, prepared according to literature (Gonzalez, J. C., Lobo-Antunes, J., Perez-Lourido, P., Santana, L., Uriarte, E. Synthesis 2002, 4:475-478). ¹H NMR (300 MHz, CDCl₃): δ, 2.50 (d, J=1.1 Hz, 3H), 6.22 (d, J=1.1 Hz, 1H), 6.97 (t, J=2.3 Hz, 1H), 7.26 (m, 1H), 7.33 (d, J=8.6 Hz, 1H), 7.40 (d, J=8.6 Hz, 1H), 8.54 (bs, 1H). MS (ACPI⁺) calculated for C₁₂H₉NO₂ m/z 199.21, observed m/z 200.2.

Compound 37 and 38

Known compounds, prepared according to literature (Chattopadhyay, S. K., Maity, S., Panja, S. Tetrahedron Lett. 2002, 43:7781-7783).

Compound 39

To a solution of 38 (324 mg, 1.50 mmol) in CH₂Cl₂ (20 mL) at RT, PhNHTf (639 mg, 1.80 mmol) was added at once. NEt₃ (417 μL, 3.0 mmol) was added dropwise over 5 min. The reaction mixture was stirred at RT for 10 hr. The solvents were removed in vacuo. The residue was purified by silica gel flash chromatography with 6:1 Hex/EtOAc to give the title compound (454 mg, 87%). ¹H NMR (400 MHz, CDCl₃): δ, 2.68 (s, 3H), 3.89 (m, 2H), 4.70 (d, J=17.4 Hz, 1H), 5.20 (d, J=10.4 Hz, 1H), 6.03 (m, 1H), 6.34 (s, 1H), 7.36 (d, J=9.2 Hz, 2H), 7.47 (d, J=9.2 Hz, 2H). ¹³C NMR (100 MHz, CDCl₃): δ, 24.1, 31.3, 117.4, 117.9, 118.8, 120.7, 124.1, 130.8, 134.4, 144.6, 152.3, 153.5, 158.8. IR υₘₐₓ(NaCl)/cm⁻¹: 1727, 1428, 1216, 1135, 965. MS (APCI⁺) calculated for C₁₄H₁₁F₃O₅S m/z 348.30, observed m/z 348.8.

Compound 40

Compound 39 (70 mg, 0.2 mmol) was dissolved in Et₂O (1 mL) at RT, OsO₄ (0.50 mL, 0.08 mmol, 2.5% in tBuOH) was added. The mixture was stirred for 10 min. H₂O (2 mL) was added, the colorless clear solution turned dark. NaIO₄ (426 mg, 2 mmol) was then added portion wise over 3 hr. EtOAc (10 mL) and H₂O (10 mL) were added. The organic layer was washed with brine, dried over magnesium sulfate and concentrated in vacuo (with caution). The residue was purified by silica gel column chromatography with 3:1 Hex/EtOAc to give title compound as a white solid (60 mg, 85%). ¹H NMR (400 MHz, CDCl₃): δ, 2.52 (d, J=1.2 Hz, 3H), 4.39 (s, 2H), 6.37 (d, J=1.2 Hz, 1H), 7.4 (d, J=9.2 Hz, 2H), 7.52 (d, J=9.2 Hz, 2H). ¹³C NMR (100 MHz, CDCl₃): δ, 24.7, 43.3, 119.4, 119.8, 121.1, 124.2, 124.5, 144.3, 151.0, 153.5, 158.4, 195.6. IR υₘₐₓ(NaCl)/cm⁻¹: 1730, 1417, 1221, 1135, 917, 830. MS (APCI⁺) calculated for C₁₃H₉F₃O₆S m/z 350.27, observed m/z 351.0.

Compound 41

Compound 40 (10 mg, 0.0286 mmol) was dissolved in CH₂Cl₂ (1 mL) at 0° C., NaBH₄ (1 mg, 0.0286 mmol) suspension in EtOH (1 mL) was added dropwise over 2 min. The reaction was complete in 5 min and quenched with 1N HCl. EtOAc (10 mL) was added. The organic layer was washed with H₂O and brine, dried over anhydrous MgSO₄, and concentrated in vacuo. The residue was purified by silica gel flash chromatography with 2:1 Hex/EtOAc to give the title compound (9.6 mg, 95%). ¹H NMR (400 MHz, CDCl₃): δ, 2.04 (bs, 1H), 2.74 (s, 3H), 3.44 (t, J=6.3 Hz, 2H), 3.86 (t, J=6.3 Hz, 2H), 6.33 (s, 1H), 7.31 (d, J=9.1 Hz, 2H), 7.45 (d, J=9.1 Hz, 2H). ¹³C NMR (100 MHz, CDCl₃): δ, 25.5, 30.5, 62.5, 117.8, 118.9, 120.8, 124.0, 130.9, 144.7, 152.71, 153.4, 158.9. IR υₘₐₓ(NaCl)/cm⁻¹: 3480, 1724, 1413, 1213, 1141, 935. MS (APCI⁺) calculated for C₁₃H₁₁F₃O₆S m/z 352.28, observed m/z 352.9.

Compound 42

A flask containing crude compound 41 (43 mg, 0.122 mmol) was flushed with Ar. Pd(OAc)₂ (1.4 mg, 0.006 mmol), Ph₂C═NH (25 μL, 0.146 mmol), Cs₂CO₃ (55 mg, 0.171 mmol) and BINAP (8 mg, 0.012 mmol) and THF (5 mL) were then added. The mixture was then stirred at reflux for 4 h under Ar. EtOAc (20 mL) and H₂O (20 mL) were added. The organic layer was washed with water and brine, dried over anhydrous magnesium sulfate, concentrated in vacuo. The residue was purified by silica gel column chromatography with 3:1 Hex/EtOAc to give the title compound as a white solid (18 mg, 72%). ¹H NMR (300 MHz, CDCl₃): δ, 1.88 (s, 3H), 2.70 (t, J=8.8 Hz, 2H), 3.49 (t, J=8.8 Hz, 2H), 6.25 (s, 1H), 6.97 (d, J=8.7 Hz, 2H), 7.13 (d, J=8.7 Hz, 2H). MS (APCI⁺) calculated for C₁₂H₁₀O₃ m/z 202.21, observed m/z 202.8.

Compound 43

To a solution of compound 41 (212 mg, 0.60 mmol) in DMF (10 mL) at RT, TBDMSCl (95 mg, 0.63 mmol) was added at once. Imidazole (86 mg, 1.26) was added portionwise over 10 min. The reaction mixture was stirred at RT for 16 hr. EtOAc (50 mL) and H₂O (50 mL) were added. The organic layer was washed with H₂O (2×30 mL) and brine, dried over anhydrous MgSO₄, and concentrated in vacuo. The residue was purified by silica gel flash chromatography with 4:1 Hex/EtOAc to give the title compound (241 mg, 86%). ¹H NMR (400 MHz, CDCl₃): δ, −0.15 (s, 6H), 0.74 (s, 9H), 2.74 (s, 3H), 3.42 (t, J=6.3 Hz, 2H), 3.80 (t, J=6.3 Hz, 2H), 6.33 (s, 1H), 7.31 (d, J=9.1 Hz, 2H), 7.44 (d, J=9.1 Hz, 2H). ¹³C NMR (100 MHz, CDCl₃): δ, −5.6, 18.1, 25.7, 15.8, 30.3, 62.8, 116.7, 117.5, 118.8, 121.2, 123.8, 131.8, 144.5, 153.1, 153.3, 158.7. IR υₘₐₓ(NaCl)/cm⁻¹: 1731, 1419, 1217, 1100, 937, 833. MS (APCI⁺) calculated for C₁₉H₂₅F₃O₆SSi m/z 466.54, observed m/z 467.2.

Compound 45

A mixture of 7-amino-4-methylcoumarin (0.53 g, 3.0 mmol), 3-chloro-3-methyl-1-butyne (268 μL, 2.4 mmol), cuprous chloride (18 mg, 0.18 mmol), copper bronze powder (14 mg, 0.21 mmol), Et₃N (1.04 mL, 7.5 mmol), water (10 mL) and THF (40 mL) was prepared. After stirring under Ar at 70° C. for 6 h, the reaction mixture was poured into 100 mL of EtOAc and 100 mL of water. The organic layer was washed successively with water and brine, dried over anhydrous MgSO₄, and concentrated in vacuo. The residue was purified by silica gel flash chromatography with 4:1 Hex/EtOAc to give the title compound (0.35 g, 52%). ¹H NMR (400 MHz, CDCl₃): δ, 1.64 (s, 6H), 2.32 (s, 3H), 2.41 (s, 1H), 4.52 (bs, 1H), 5.98 (s, 1H), 6.73 (d, J=8.6 Hz, 1H), 6.95 (s, 1H), 7.35 (d, J=8.3 Hz, 1H). ¹³C NMR (100 MHz, CDCl₃): δ, 18.6, 30.2, 47.8, 71.4, 86.1, 101.0, 109.7, 110.8, 112.4, 124.8, 148.6, 152.8, 154.9, 161.7. IR υₘₐₓ(NaCl)/cm⁻¹: 3343, 1700, 1612, 1558, 1397, 1217. HRMS (FAB⁺) calculated for C₁₅H₁₆NO₂ m/z 242.1181, observed m/z 242.1185.

Compound 46

A mixture of compound 45 (80 mg, 0.33 mmol), and 5% Pd/BaSO₄ (3 mg) in Et₂O (6 mL) and MeOH (6 mL) was stirred under a H₂ atmosphere at room temperature for 20 h. The reaction mixture was filtered through Celite and purified by flash chromatography with 5:1 Hex/EtOAc to give the title compound as a yellow solid (54 mg, 67% yield). ¹H NMR (400 MHz, CDCl₃): δ, 1.42 (s, 6H), 2.30 (d, J=1.0 Hz, 3H), 2.41 (s, 1H), 4.46 (bs, 1H), 5.15 (d, J=10.7 Hz, 1H), 5.20 (d, J=17.6 Hz, 1H), 5.93 (dd, J=10.7, 17.5 Hz, 1H), 5.93 (s, 1H), 6.54 (dd, J=2.3, 8.7 Hz, 1H), 6.60 (d, J=2.3 Hz, 1H), 7.28 (d, J=8.7 Hz, 1H). ¹³C NMR (100 MHz, CDCl₃): δ, 18.6, 28.1, 54.5, 100.4, 109.1, 110.1, 112.3, 113.7, 124.7, 144.1, 149.6, 152.7, 155.0, 161.8. IR υₘₐₓ(NaCl)/cm⁻¹: 3356, 1700, 1616, 1558, 1397, 1157, 837. HRMS (FAB⁺) calculated for C₁₅H₁₈NO₂ M/Z 244.1338, observed m/z 244.1332.

Compound 47

A solution of compound 46 (55 mg, 0.21 mmol) and p-toluenesulfonic acid monohydrate (10 mol %) in MeCN/H₂O (10 mL/1 mL) was heated for 4 h at 65° C. MeCN was removed from the reaction mixture in vacuo; the residue was extracted with EtOAc (2×30 mL), and washed with brine. The organic layer was dried over anhydrous MgSO₄, and concentrated in vacuo. The residue was purified by silica gel flash chromatography with 4:1 Hex/EtOAc to give the title compound as a yellow solid (34 mg, 66%). ¹H NMR (400 MHz, CDCl₃): δ, 1.73 (s, 3H), 1.85 (s, 3H), 2.37 (s, 3H), 3.25 (d, J=6.5 Hz, 2H) 4.16 (bs, 2H), 5.08 (t, J=6.4 Hz, 1H), 6.04 (s, 1H), 6.62 (d, J=8.5 Hz, 1H), 7.30 (d, J=8.3 Hz, 1H). ¹³C NMR (100 MHz, CDCl₃): δ, 18.6, 28.1, 54.5, 100.4, 109.1, 110.1, 112.3, 113.7, 124.7, 144.1, 149.6, 152.7, 155.0, 161.8. IR υₘₐₓ(NaCl)/cm⁻¹: 3356, 1700, 1616, 1558, 1397, 1157, 837. HRMS (FAB⁺) calculated for C₁₅H₁₇NO₂ m/z 243.1259, observed m/z 243.1258.

Compound 48

To a solution of compound 47 (140 mg, 0.61 mmol) in CH₂Cl₂ (10 mL) at RT were K₂CO₃ (250 mg, 1.83 mmol) and TrocCl (180 μL, 1.22 mmol) added under Ar. The reaction mixture was stirred at RT for 10 h. EtOAc (30 mL) and H₂O (30 mL) were added. The organic layer was separated, washed with brine, dried over anhydrous MgSO₄, and concentrated in vacuo. The residue was purified by silica gel flash chromatography with 6:1 Hex/EtOAc to give the title compound as a white solid (198 mg, 78%). ¹H NMR (400 MHz, CDCl₃): δ, 1.42 (s, 6H), 2.30 (d, J=1.0 Hz, 3H), 2.41 (s, 1H), 4.46 (bs, 1H), 5.15 (d, J=10.7 Hz, 1H), 5.20 (d, J=17.6 Hz, 1H), 5.93 (dd, J=10.7, 17.5 Hz, 1H), 5.93 (s, 1H), 6.54 (dd, J=2.3, 8.7 Hz, 1H), 6.60 (d, J=2.3 Hz, 1H), 7.28 (d, J=8.7 Hz, 1H). ¹³C NMR (100 MHz, CDCl₃): δ, 18.6, 28.1, 54.5, 100.4, 109.1, 110.1, 112.3, 113.7, 124.7, 144.1, 149.6, 152.7, 155.0, 161.8. IR υₘₐₓ(NaCl)/cm⁻¹: 3356, 1700, 1616, 1558, 1397, 1157, 837. HRMS (FAB⁺) calculated for C₁₈H₁₈Cl₃NO₄ m/z 417.0301, observed m/z 417.0286.

Compound 49

Compound 48 (73 mg, 0.175 mmol) was dissolved in Et₂O (1 mL) at RT, OsO₄ (0.44 mL, 0.035 mmol, 2.5% in ^{t}BuOH) was added. The mixture was stirred for 10 min. H₂O (2 mL) was added, the colorless clear solution turned dark. NaIO₄ (372 mg, 1.75 mmol) was then added portionwise over 3 h. EtOAc (10 mL) and H₂O (10 mL) were added. The organic layer was washed with brine, dried over magnesium sulfate and concentrated in vacuo (with caution). The residue was purified by silica gel column chromatography with 2:1 Hex/EtOAc to give title compound as a white solid (44 mg, ˜65%). ¹H NMR (400 MHz, CDCl₃): δ, 2.41 (s, 3H), 3.28 (d, J=17.7 Hz, 1H), 3.53 (dd, J=7.5, 17.9 Hz, 1H), 4.88 (d, J=11.8 Hz, 1H), 4.99 (s, 1H), 6.18 (s, 1H), 6.24 (m, 1H), 7.51 (d, J=8.6 Hz, 1H). ¹³C NMR (100 MHz, CDCl₃): δ, 18.9, 33.4, 74.8, 84.5, 85.1, 110.9, 111.3, 113.1, 115.3, 116.3, 125.2, 143.8, 152.8, 160.6. IR υₘₐₓ(NaCl)/cm⁻¹: 3378, 1719, 1618, 1558, 1388, 1269, 11367, 1031. MS (APCI⁺) calculated for C₁₅H₁₂Cl₃NO₅ m/z 392.62, observed m/z 394.1.

Compound 51

To a solution of 5-hydroxyindole (106 mg, 0.8 mmol) and but-2-ynoic acid (74 mg, 0.88 mmol) in CH₂Cl₂ (10 mL) and DMF (1 mL) at RT were DCC (165 mg, 0.8 mmol) and DMAP (cat) added. The solvents were removed under vacuum; the residue was suspended in EtOAc (20 mL). White DCU was filtered and the filtrate was washed with brine, dried over anhydrous MgSO₄, and concentrated in vacuo. The residue was purified by silica gel flash chromatography with 4:1 Hex/EtOAc to give the title compound as a white solid (87 mg, 55%). ¹H NMR (400 MHz, CDCl₃): δ, 2.05 (s, 3H), 6.52 (m, 1H), 6.93 (dd, J=2.2, 8.7 Hz, 1H), 7.20 (t, J=2.8 Hz, 1H), 7.30 (d, J=8.7 Hz, 1H), 7.38 (d, J=2.2 Hz, 1H), 8.25 (bs, 1H). ¹³C NMR (100 MHz, CDCl₃): δ, 4.2, 72.4, 87.7, 102.8, 111.4, 112.4, 115.5, 125.5, 127.9, 133.6, 143.5, 152.8. IR υₘₐₓ(NaCl)/cm⁻¹: 3411, 1700, 1260, 1127, 1043, 884. HRMS (FAB⁺) calculated for C₁₂H₉NO₂ m/z 199.0633, observed m/z 199.0633.

Compound 52

A solution of compound 51 (100 mg, 0.5 mmol) and PtO₂ (8.5 mg, 5 mol %) in dioxane/ClCH₂CH₂Cl (5 mL/5 mL) was heated for 3 h at 70° C. EtOAc (30 mL) and H₂O (10 mL) were added, the organic layer was separated, washed with brine, dried over anhydrous MgSO₄, and concentrated in vacuo. The residue was purified by silica gel flash chromatography with 1:1 Hex/EtOAc to give the title compound as a white solid (54 mg, 54%). ¹H NMR (400 MHz, DMSO-d₆): δ, 2.69 (s, 3H), 6.31 (s, 1H), 6.88 (s, 1H), 7.14 (d, J=8.7 Hz, 1H), 7.58 (t, J=2.8 Hz, 1H), 7.68 (d, J=8.7 Hz, 1H), 11.6 (bs, 1H). ¹³C NMR (100 MHz, DMSO-d₆): δ, 22.5, 102.2, 110.2, 111.2, 112.5, 115.9, 122.3, 127.3, 132.4, 148.9, 154.6, 160.0. IR υₘₐₓ(NaCl)/cm⁻¹: 3171, 1664, 1588, 1352, 1171, 886. HRMS (FAB⁺) calculated for C₁₂H₁₀NO₂ m/z 200.0712, observed m/z 200.0705.

Compound 54

Prepared from 6-hydroxyindole and but-2-ynoic acid in a same manner as compound 51. ¹H NMR (400 MHz, CDCl₃): δ, 2.06 (s, 3H), 6.50 (s, 1H), 6.90 (dd, J=2.0, 8.5 Hz, 1H), 7.08 (t, J=3.2 Hz, 2H), 7.61 (d, J=8.5 Hz, 1H), 8.33 (bs, 1H). ¹³C NMR (100 MHz, CDCl₃): δ, 4.1, 72.2, 88.1, 102.1, 103.8, 113.3, 120.8, 125.2, 125.9, 135.1, 145.1, 152.8. IR υₘₐₓ(NaCl)/cm⁻¹: 3427, 2236, 1702, 1252, 1135, 1040, 796. MS (APCI⁺) calculated for C₁₂H₉NO₂ m/z 199.21, observed m/z 200.1.

Compound 55

Prepared from compound 54 in a same manner as compound 52. ¹H NMR (400 MHz, DMSO-d₆): δ, 2.74 (d, J=0.9 Hz, 3H), 6.28 (d, J=0.9 Hz, 1H), 6.61 (m, 1H), 7.06 (d, J=8.6 Hz, 1H), 7.41 (t, J=2.8 Hz, 1H), 7.79 (d, J=8.6 Hz, 1H), 11.1 (bs, 1H). ¹³C NMR (100 MHz, DMSO-d₆): δ, 22.5, 102.2, 106.0, 108.9, 112.2, 124.4, 125.1, 126.2, 129.7, 150.3, 152.5, 159.8. IR υₘₐₓ(NaCl)/cm⁻¹: 3349, 1705, 1685, 1579, 1342, 1187, 807. MS (FAB⁺) calculated for C₁₂H₁₀NO₂ m/z 199.21, observed m/z 200.1.

Compound 57

Prepared from Boc deprotection of compound 71 in a same manner as compound 3. ¹H NMR (400 MHz, CD₃OD): δ, 2.68 (d, J=1.1 Hz, 1H), 3.37 (t, J=6.0 Hz, 2H), 3.93 (t, J=6.0 Hz, 2H), 6.37 (d, J=1.1 Hz, 1H), 7.29 (d, J=8.8 Hz, 1H), 7.42 (d, J=8.8 Hz, 1H). ¹³C NMR (100 MHz, CD₃OD): δ, 25.7, 31.4, 63.6, 118.3, 119.2, 121.3, 125.6, 130.9, 134.9, 154.0, 155.5, 161.7. IR υₘₐₓ(NaCl)/cm⁻¹: 3341, 2890, 1685, 1204, 1056. HRMS (FAB⁺) calculated for C₁₂H₁₃NO₃ m/z 219.24, observed m/z 220.1.

Compound 58

Prepared from catalytic hydrogenation of compound 80 in a same manner as compound 8. ¹H NMR (300 MHz, CD₃OD): δ, 2.63 (t, J=6.0 Hz, 2H), 3.82 (t, J=6.0 Hz, 2H), 6.29 (d, J=8.8 Hz, 1H), 6.87 (s, 1H), 7.07 (s, 1H), 7.77 (d, J=8.8 Hz, 1H). ¹³C NMR (100 MHz, CD₃OD): δ, 35.8, 62.2, 113.3, 115.9, 116.4, 119.0, 132.0, 144.4, 145.3, 147.9, 163.4. IR υₘₐₓ(NaCl)/cm⁻¹: 3316, 1686, 1555, 1428, 1288, 1106, 1048. MS (APCI⁺) calculated for C₁₁H₁₁NO₃ m/z 205.21, observed m/z 206.1.

Compound 59

Compound 79 (35 mg, 0.15 mmol) in EtOH (5 mL) and EtOAc (5 mL) was hydrogenated over PtO₂ at room temperature under an atmospheric pressure of hydrogen for 2 h. The reaction mixture was concentrated in vacuo and the residue was purified by silica gel column chromatography with 1:1 Hex/EtOAc to give the title compound (23 mg, 83%). ¹H NMR (300 MHz, CD₃OD): δ, 6.36 (d, J=9.4 Hz, 1H), 6.61 (m, 1H), 7.58 (m, 2H), 7.70 (s, 1H), 8.09 (d, J=9.4 Hz, 1H). ¹³C NMR (75 MHz, CD₃OD): δ, 22.5, 102.2, 106.0, 108.9, 112.2, 124.4, 125.1, 126.2, 129.7, 150.3, 152.5, 159.8. IR υₘₐₓ(NaCl)/cm⁻¹: 3276, 1688, 1630, 1508, 1313, 1142. MS (APCI⁺) calculated for C₁₁H₇NO₂ M/Z 185.18, observed m/z 186.1.

Compound 60, 61 and 62.

Known compounds, prepared according to literature (Lin, S. T., Yang, F. M., Yang, H. Y., Huang, K. F. J. Chem. Research (S) 1995, 372-373).

Compound 63

Prepared from compound 62 in a same manner as compound 45. ¹H NMR (400 MHz, CDCl₃): δ, 1.62 (s, 6H), 2.39 (d, J=1.2 Hz, 3H), 2.41 (s, 1H), 3.77 (bs, 1H), 6.26 (d, J=1.1 Hz, 1H), 7.09 (dd, J=2.7, 8.9 Hz, 1H), 7.16-7.20 (m, 2H). ¹³C NMR (100 MHz, CDCl₃): δ, 18.9, 30.4, 48.6, 71.1, 87.2, 110.6, 114.9, 117.2, 120.0, 121.7, 141.7, 146.9, 152.0, 161.0. IR υₘₐₓ(NaCl)/cm⁻¹: 3359, 1695, 1575, 1496, 1363, 1183. MS (APCI⁺) calculated for C₁₅H₁₆NO₂ m/z 241.29, observed m/z 242.2.

Compound 64

Prepared from compound 63 in a same manner as compound 46. ¹H NMR (400 MHz, CDCl₃): δ, 1.39 (s, 6H), 2.30 (d, J=1.1 Hz, 3H), 3.83 (s, 1H), 5.16 (m, 2H), 5.99 (dd, J=10.6, 17.5 Hz, 1H), 6.82-6.88 (m, 2H), 7.08 (d, J=9.0 Hz, 1H). ¹³C NMR (100 MHz, CDCl₃): δ, 18.8, 28.0, 54.9, 108.8, 112.9, 114.7, 116.9, 119.8, 120.4, 142.9, 145.6, 145.8, 152.1, 161.1. IR υₘₐₓ(NaCl)/cm⁻¹: 3353, 1698, 1575, 1536, 1181, 935. MS (APCI⁺) calculated for C₁₅H₁₇NO₂ m/z 243.30, observed m/z 244.2.

Compound 65

Prepared from compound 64 in a same manner as compound 47. ¹H NMR (400 MHz, CDCl₃): δ, 1.71 (d, J=1.4 Hz, 3H), 1.75 (s, 3H), 2.56 (d, J=1.1 Hz, 3H), 3.51 (d, J=5.5 Hz, 2H) 3.65 (bs, 2H), 5.00 (m, 1H), 6.15 (d, J=1.1 Hz, 1H), 6.87 (d, J=8.7 Hz, 1H), 7.01 (d, J=8.7 Hz, 1H). ¹³C NMR (100 MHz, CDCl₃): δ, 18.3, 24.9, 25.6, 28.2, 115.8, 116.9, 119.1, 119.9, 120.7, 122.4, 135.1, 142.2, 148.0, 153.3, 160.4. IR υₘₐₓ(NaCl)/cm⁻¹: 3533, 1706, 1569, 1436, 1361. MS (APCI⁺) calculated for C₁₅H₁₇NO₂ m/z 243.30, observed m/z 244.2.

Compound 66

Phthalic anhydride (287 mg, 1.94 mmol) and triethylamine (829 μL, 5.96 mmol) was added to a solution of compound 65 (363 mg, 1.49 mmol) in toluene (25 mL). The reaction mixture was refluxed under Ar for 12 h. After the solvent was removed in vacuo, the residue was purified by silica gel column chromatography with 4:1 Hex/EtOAc to give the title compound (468 mg, 84%). ¹H NMR (400 MHz, CDCl₃): δ, 1.32 (d, J=0.8 Hz, 3H), 1.50 (d, J=1.4 Hz, 3H), 2.64 (d, J=0.8 Hz, 3H), 3.60 (m, 2H) 4.96 (m, 1H), 6.28 (d, J=0.8 Hz, 1H), 7.30 (d, 13.5 Hz, 1H), 7.32 (d, J=13.5 Hz, 1H), 7.79 (m, 2H), 7.92 (m, 2H). ¹³C NMR (100 MHz, CDCl₃): δ, 18.0, 24.7, 25.2, 29.5, 116.9, 118.1, 120.4, 121.9, 123.5, 128.4, 131.9, 132.1, 133.0, 134.2, 140.1, 152.7, 155.2, 159.0, 167.0. IR υₘₐₓ(NaCl)/cm⁻¹: 1713, 1384, 1215, 1105, 720. MS (APCI⁺) calculated for C₂₃H₁₉NO₄ m/z 373.40, observed m/z 374.3.

Compound 67

To a solution of compound 65 (421 mg, 1.13 mmol) in CH₂Cl₂ (205 mL) at −78° C. was bubbled with O₃/O₂ (from O₃ generator) for about 10 min until a blue color appeared. The reaction mixture was warmed up to RT and Me₂S (1.24 mL, 17 mmol) added. The mixture was stirred at RT Ar for 12 h. After the solvent was removed in vacuo, the residue was purified by silica gel column chromatography with 2:1 Hex/EtOAc to give the title compound (322 mg, 82%). ¹H NMR (400 MHz, CDCl₃): δ, 2.54 (s, 3H), 3.03 (s, 2H), 6.34 (s, 1H), 7.45 (m, 4H), 7.84 (m, 2H), 7.95 (m, 2H), 9.63 (s, 1H). ¹³C NMR (100 MHz, CDCl₃): δ, 25.5, 44.7, 118.6, 118.7, 120.9, 124.0, 128.7, 130.7, 131.2, 131.9, 134.7, 151.6, 154.9, 158.8, 167.0, 197.1. IR υₘₐₓ(NaCl)/cm⁻¹: 1713, 1384, 1215, 1105, 720. MS (APCI⁺) calculated for C₂₀H₁₃NO₅ m/z 347.32, observed m/z 348.2.

Compound 68

Prepared from compound 67 in a same manner as of compound 41. ¹H NMR (300 MHz, CDCl₃): δ, 1.78 (s, 1H), 2.72 (d, J=0.8 Hz, 3H), 2.43 (t, J=7.0 Hz, 2H), 3.70 (t, J=7.0 Hz, 2H), 6.34 (d, J=0.8 Hz, 1H), 7.34 (d, J=8.7 Hz, 2H), 7.37 (d, J=8.7 Hz, 2H), 7.83 (m, 2H), 7.96 (m, 2H). MS (APCI⁺) calculated for C₂₀H₁₅NO₅ m/z 349.34, observed m/z 350.2.

Compound 69

Boc₂O (646 mg, 3.0 mmol) and DMAP (cat) were added to a solution of compound 65 (120 mg, 0.50 mmol) in THF (10 mL). The reaction mixture was refluxed under Ar for 20 h. After the solvent was removed in vacuo, the residue was purified by silica gel column chromatography with 5:1 Hex/EtOAc to give the title compound (202 mg, 91%). ¹H NMR (400 MHz, CDCl₃): δ, 1.41 (s, 18H), 1.68 (d, J=1.4 Hz, 3H), 1.71 (d, J=0.9 Hz, 3H), 2.66 (d, J=1.2 Hz, 3H), 3.61 (m, 2H), 4.97 (m, 1H), 6.28 (d, J=1.2 Hz, 1H), 7.26 (m, 2H). ¹³C NMR (100 MHz, CDCl₃): δ, 18.3, 24.6, 25.6, 28.0, 28.5, 116.5, 117.4, 119.4, 122.0, 131.8, 132.9, 135.5, 138.0, 151.1, 153.5, 154.2, 159.7. IR υₘₐₓ(NaCl)/cm⁻¹: 1738, 1367, 1273, 1154, 1117. MS (APCI⁺) calculated for C₂₅H₃₃NO₆m/z 443.53, observed m/z 444.3.

Compound 70

Prepared from compound 69 in a same manner as of compound 67. ¹H NMR (400 MHz, CDCl₃): δ, 1.42 (s, 18H), 2.51 (d, J=1.2 Hz, 3H), 4.10 (d, J=1.1 Hz, 2H), 6.32 (d, J=1.2 Hz, 1H), 7.37 (s, 2H), 9.17 (s, 1H). ¹³C NMR (100 MHz, CDCl₃): δ, 25.3, 27.9, 44.1, 83.3, 118.1, 118.4, 120.3, 129.6, 131.8, 135.5, 136.1, 151.1, 151.9, 154.0, 159.2, 197.3. IR υₘₐₓ(NaCl)/cm⁻¹: 1732, 1369, 1274, 1153, 1112. MS (APCI⁺) calculated for C₂₂H₂₇NO₇ m/z 417.45, observed m/z 418.4.

Compound 71

Prepared from compound 70 in a same manner as of compound 41. ¹H NMR (300 MHz, CDCl₃): δ, 1.44 (s, 18H), 2.01 (bs, 1H), 2.69 (s, 3H), 3.26 (t, J=7.0 Hz, 2H), 3.76 (t, J=7.0 Hz, 2H), 7.28 (m, 2H). ¹³C NMR (75 MHz, CDCl₃): δ, 25.3, 27.9, 31.8, 62.6, 83.8, 117.3, 118.2, 120.0, 131.9, 135.5, 135.9, 151.9, 153.3, 154.4, 159.8. IR υₘₐₓ(NaCl)/cm⁻¹: 1732, 1369, 1274, 1153, 1112. MS (APCI⁺) calculated for C₂₂H₂₉NO₇ m/z 419.47, observed m/z 420.3.

Compound 72

A flask charged with Pd(PPh₃)₄ (216 mg, 0.19 mmol), LiCl (0.78 g, 18.7 mmol), and compound 11 (1.44 g, 4.67 mmol) was flushed with Ar. Anhydrous THF (25 mL), tributylallyltin (1.74 mL, 5.60 mmol) was then added. After being stirred at 80° C. for 24 h, the product was extracted with EtOAc, washed with water (3×50 mL) and brine, dried over anhydrous MgSO₄, concentrated in vacuo. The residue was purified by silica gel column chromatography with 6:1 Hex/EtOAc to give the title compound (896 mg, 96%). ¹H NMR (400 MHz, CDCl₃): δ, 2.38 (d, J=1.2 Hz, 3H), 3.43 (d, J=6.6 Hz, 2H), 5.08 (m, 1H), 5.11 (s, 1H), 5.91 (m, 1H), 6.19 (d, J=1.0 Hz, 1H), 7.10 (m, 2H), 7.48 (m, 1H). ¹3C NMR (100 MHz, CDCl₃): δ, 18.6, 39.9, 114.0, 116.5, 116.7, 117.8, 124.2, 124.5, 135.5, 144.6, 151.9, 153.3, 160.5. IR υₘₐₓ(NaCl)/cm⁻¹: 1731, 1618, 1388, 1147, 879, 738. MS (APCI⁺) calculated for C₁₃H₁₂O₂ m/z 200.23, observed m/z 201.1.

Compound 73

To a solution of compound 72 (95 mg, 0.475 mmol) in conc. H₂SO₄ (1 mL) at 0° C. was KNO₃ (58 mg, 0.57 mmol) added portionwise. The reaction mixture was stirred at 0° C. for 1 h and poured onto ice. The product was extracted with EtOAc, washed with brine, and dried over MgSO₄. After the solvent was removed in vacuo, the residue was purified by silica gel column chromatography with 2:1 Hex/EtOAc to give the title compound (70 mg, 60%). ¹H NMR (400 MHz, CDCl₃): δ, 1.39 (d, J=6.2 Hz, 3H), 1.83 (bs, 1H), 2.53 (d, J=1.2 Hz, 3H), 3.10 (dd, J=8.4, 13.6 Hz, 1H), 3.31 (dd, J=3.7, 13.6 Hz, 1H), 4.18 (m, 1H), 6.41 (d, J=1.1 Hz, 1H), 7.40 (s, 1H), 8.28 (s, 1H). IR υₘₐₓ(NaCl)/cm⁻¹: 3460, 1721, 1626, 1351, 1158, 1056. MS (APCI⁺) calculated for C₁₃H₁₂O₂ m/z 247.25, observed m/z 248.2.

Compound 74

Prepared from compound 73 in a same manner as compound 67. ¹H NMR (400 MHz, CDCl₃): δ, 2.44 (d, J=1.2 Hz, 3H), 3.82 (d, J=1.8 Hz, 2H), 6.30 (d, J=1.2 Hz, 1H), 7.15 (dd, J=1.7, 8.0 Hz, 1H), 7.21 (d, J=1.4 Hz, 1H), 7.60 (d, J=8.0 Hz, 1H). IR υₘₐₓ(NaCl)/cm⁻¹: 1731, 1620, 1389, 1148, 876. MS (APCI⁺) calculated for C₁₂H₁₀O₃ m/z 202.21, observed m/z 203.1.

Compound 75

Prepared from compound 74 in a same manner as compound 41. ¹H NMR (300 MHz, CDCl₃): δ, 2.44 (d, J=1.2 Hz, 3H), 2.98 (t, J=6.4 Hz, 2H), 3.94 (t, J=6.4 Hz, 2H), 6.26 (d, J=1.2 Hz, 1H), 7.16-7.27 (m, 2H), 7.55 (d, J=8.0 Hz, 1H). IR υₘₐₓ(NaCl)/cm⁻¹: 3450, 1710, 1621, 1195, 1046, 884. MS (APCI⁺) calculated for C₁₂H₁₂O₃ M/Z 204.22, observed m/z 205.0.

Compound 77

Known compound, prepared according to literature (Rozhkov, R. V., Larock, R. C. Org. Lett. 2003, 5:797-800.)

Compound 78 and 79

410 mg (2.0 mmol) of compound 77 and 0.40 mL (3.0 mmol) of N,N-Dimethylformamide dimethyl acetal was refluxed under Ar in 10 ml of anhydrous DMF for 1 hr. The reaction mixture was concentrated in vacuo to give crude 78. 20 ml of Et₂O and 20 ml of 12 N aq. HCl was added and the mixture was refluxed for 1 hr. 50 mL of Et₂O and 50 mL of water were added. The organic layer was washed with brine, dried over anhydrous MgSO₄, and concentrated in vacuo. The residue was purified by silica gel flash chromatography with 2:1 Hex/EtOAc to give the title compound (0.35 g, 52%). ¹H NMR (300 MHz, DMSO-d₆): δ, 4.33 (s, 2H), 6.66 (d, J=9.6 Hz, 1H), 7.56 (s, 1H), 8.18 (d, J=9.6 Hz, 1H), 8.62 (s, 1H), 9.75 (s, 1H). ¹³C NMR (75 MHz, DMSO-d₆): δ, 47.7, 118.0, 118.2, 121.1, 125.6, 133.1, 142.9, 144.6, 155.7, 158.9, 198.2. IR υₘₐₓ(NaCl)/cm⁻¹: 1739, 1623, 1530, 1326, 1269, 1110. MS (APCI⁺) calculated for C₁₁H₇NO₅m/z 233.18, observed m/z 234.2.

Compound 80

Prepared from compound 79 in a same manner as compound 41. ¹H NMR (300 MHz, CDCl₃): δ, 3.29 (t, J=6.1 Hz, 2H), 3.99 (m, 2H), 6.52 (d, J=9.6 Hz, 1H), 7.37 (s, 1H), 7.72 (d, J=9.6 Hz, 1H), 8.17 (s, 1H). ¹³C NMR (75 MHz, CDCl₃): δ, 35.5, 60.5, 117.1, 117.3, 119.6, 124.7, 138.2, 142.6, 145.2, 154.3, 158.6. IR υₘₐₓ(NaCl)/cm⁻¹: 3324, 3224, 1736, 1627, 1530, 1366, 1109, 1050. MS (APCI⁺) calculated for C₁₁H₉NO₅ m/z 235.19, observed m/z 236.1.

Compound 81

To a solution of compound 110 (10 mg, 0.04 mmol) in MeOH (1.5 mL) and H₂O (1.5 mL) was KNO₃ (20 mg, 0.16 mmol) added. The reaction was stirred at RT for 1 h. EtOAc (10 mL) and H₂O (10 mL) were added and the organic layer washed with brine, dried over anhydrous MgSO₄, and concentrated in vacuo. The residue was purified by silica gel flash chromatography with 1:1 Hex/EtOAc to give the title compound (6.7 mg, 82%). ¹H NMR (300 MHz, DMSO-d₆): δ, 2.63 (t, J=6.8 Hz, 2H), 3.56 (t, J=6.8 Hz, 2H), 6.37 (d, J=9.5 Hz, 1H), 6.69 (s, 1H), 6.78 (s, 1H), 7.89 (d, J=9.4 Hz, 1H). ¹³C NMR (75 MHz, DMSO-d₆): δ, 38.6, 62.0, 115.0, 115.4, 117.3, 118.4, 135.8, 139.3, 145.1, 160.2. IR υₘₐₓ(NaCl)/cm⁻¹: 3473, 1705, 1605, 1415, 1156, 1307, 852. MS (APCI⁺) calculated for C₁₁H₁₁NO₃ m/z 205.21, observed m/z 206.2.

Compound 84

Prepared from compound 90 in a same manner as compound 39. ¹H NMR (400 MHz, CDCl₃): δ, 2.36 (d, J=1.2 Hz, 3H), 6.46 (d, J=1.2 Hz, 1H), 7.56 (d, J=9.3 Hz, 1H), 7.66 (d, J=9.3 Hz, 1H). ¹³C NMR (100 MHz, CDCl₃): δ, 18.5, 113.2, 116.5, 119.7, 120.2, 120.4, 127.0, 136.1, 147.4, 152.3, 157.0. IR υₘₐₓ(NaCl)/cm⁻¹: 1752, 1551, 1436, 1124, 1134, 955. MS (APCI⁺) calculated for C₁₁H₆F₃NO₇S m/z 353.23, observed m/z 354.2.

Compound 85

Known compounds, prepared according to literature (Rozhkov, R. V., Larock, R. C. Org. Lett. 2003, 5:797-800).

Compound 86

To a solution of compound 108 (204 mg, 1.0 mmol) in THF (20 mL) at 0° C. was BH₃ (2.1 mL, 2.1 mmol, 1 M in THF) added dropwise. The reaction mixture was stirred at 0° C. under Ar for 4 h and quenched with 0.5 N HCl. The product was extracted with EtOAc, washed with brine, and dried over MgSO₄. After the solvent was removed in vacuo, the residue was purified by silica gel column chromatography with 1:1 Hex/EtOAc to give the title compound (160 mg, 84%). ¹H NMR (400 MHz, CDCl₃): δ, 1.69 (bs, 1H), 2.91 (t, J=6.5 Hz, 2H), 3.89 (t, J=6.5 Hz, 2H), 6.39 (d, J=9.5 Hz, 1H), 7.26 (m, 1H), 7.34 (d, J=1.9 Hz, 1H), 7.40 (dd, J=1.9, 8.4 Hz, 1H), 7.66 (d, J=9.5 Hz, 1H). ¹³C NMR (100 MHz, CDCl₃): δ, 38.3, 63.3, 116.5, 116.8, 118.6, 127.8, 132.4, 135.0, 143.1, 152.4, 160.6. IR υₘₐₓ(NaCl)/cm⁻¹: 3445, 1709, 1572, 1437, 1110, 824. MS (APCI⁺) calculated for C₁₁H₁₀O₃ m/z 190.20, observed m/z 191.1.

Compound 87

Known compound, prepared according to literature (Rozhkov, R. V., Larock, R. C. Org. Lett. 2003, 5:797-800).

Compound 88

Prepared from compound 87 in a same manner as compound 39. ¹H NMR (400 MHz, CDCl₃): δ, 2.64 (d, J=1.2 Hz, 3H), 6.36 (d, J=1.2 Hz, 1H), 7.28-7.52 (m, 3H), 7.58 (t, J=8.4 Hz, 1H). ¹³C NMR (100 MHz, CDCl₃): δ, 23.3, 117.6, 118.2, 123.3, 127.1, 129.4, 131.3, 145.2, 149.7, 154.6, 158.7. IR υₘₐₓ(NaCl)/cm⁻¹: 1764, 1425, 1220, 1133, 994, 853. MS (APCI⁺) calculated for C₁₁H₆F₃NO₇S m/z 308.23, observed m/z 309.2.

Compound 89

Known compound, prepared according to literature (Ganguly, N. C.; Datta, M.; De, P.; Chakravarty, R. Synth. Comm. 2003, 33:647-659).

Compound 90

A solution of compound 89 (47 mg, 0.2 mmol) in HBr (5 mL, 48%) was refluxed at 140° C. under Ar for 3 h. H₂O (20 mL) was added and the product was extracted with EtOAc, washed with brine, and dried over MgSO₄. After the solvent was removed in vacuo, the residue was purified by silica gel column chromatography with 5% MeOH in CH₂Cl₂ to give the title compound (37 mg, 85%). ¹H NMR (400 MHz, CDCl₃): δ, 2.22 (d, J=1.1 Hz, 3H), 6.53 (d, J=1.1 Hz, 1H), 7.32 (d, J=9.1 Hz, 1H), 7.47 (d, J=9.1 Hz, 1H), 11.2 (bs, 1H). 1³C NMR (100 MHz, CDCl₃): δ, 17.5, 111.4, 118.4, 119.6, 120.5, 134.6,145.3, 147.6, 158.0. IR υₘₐₓ(NaCl)/cm⁻¹: 3302, 1734, 1664, 1558, 1351, 1288, 830. MS (APCI⁺) calculated for C₁₀H₇F₃NO₅m/z 221.17, observed m/z 221.9.

Compound 93

A suspension of compound 111 (67 mg, 0.2 mmol) in EtOH (2 mL), con. HCl (5 mL), and H₂O (5 mL) was refluxed at 110° C. under Ar for 1 h. H₂O (20 mL) was added and the product was extracted with EtOAc, washed with brine, and dried over MgSO₄. After the solvent was removed in vacuo, the residue was purified by silica gel column chromatography with Hex/EtOAc (1:1) to give the title compound (39 mg, 83%). ¹H NMR (400 MHz, CDCl₃): δ, 4.26 (s, 2H), 6.65 (d, J=9.6 Hz, 1H), 7.90 (d, J=1.7 Hz, 1H), 8.07-8.17 (m, 2H), 9.74 (bs, 1H). ¹³C NMR (100 MHz, CDCl₃): δ, 47.7, 117.6, 120.6, 128.4, 129.7, 134.9, 136.4, 143.6, 144.8, 158.0, 199.6. IR υₘₐₓ(NaCl)/cm⁻¹: 3448, 1746, 1716, 1526, 1350, 1160, 1111. MS (APCI⁺) calculated for C₁₁H₇NO₅ m/z 233.18, observed m/z 234.1.

Compound 94

Prepared from compound 109 in a same manner as compound 93. ¹H NMR (400 MHz, CDCl₃): δ, 2.94 (t, J=6.3 Hz, 2H), 3.82 (t, J=6.4 Hz, 2H), 6.54 (d, J=8.7 Hz, 1H), 7.80 (d, J=2.0 Hz, 1H), 7.98 (d, J=8.7 Hz, 1H), 8.06 (d, J=2.0 Hz, 1H). ¹³C NMR (100 MHz, CDCl₃): δ, 38.7, 61.5, 118.2, 121.8, 128.5, 134.3, 137.5, 144.4, 145.7, 159.8. IR υₘₐₓ(NaCl)/cm⁻¹: 3390, 1747, 1528, 1359, 1156, 1048, 838. MS (APCI⁺) calculated for C₁₁H₉NO₅ m/z 235.19, observed m/z 236.1.

Compound 97

Prepared from compound 96 in a same manner as compound 69. ¹H NMR (400 MHz, CDCl₃): δ, 1.36 (s, 18H), 2.23 (s, 3H), 6.43 (d, J=9.7 Hz, 1H), 7.21 (d, J=8.6 Hz, 1H), 7.38 (d, J=8.6 Hz, 1H), 7.68 (d, J=9.8 Hz, 1H). ¹³C NMR (100 MHz, CDCl₃): δ, 17.2, 27.9, 83.4, 116.2, 116.6, 117.2, 131.7, 133.1, 134.6, 138.1, 150.3, 152.3, 159.9. IR υₘₐₓ(NaCl)/cm⁻¹: 1791, 1724, 1370, 1280, 1152, 1102, 849. MS (APCI⁺) calculated for C₁₁H₉NO₅ m/z 375.42, observed m/z 376.3.

Compound 98

To a solution of compound 97 (180 mg, 0.48 mmol) in CCl₄ (5 mL) at RT were NBS (188 mg, 1.06 mmol) and benzoyl peroxide (30 mg, cat) added. The reaction mixture was refluxed under Ar for 12 h. The solvent was removed in vacuo. EtOAc (15 mL) and H₂O (15 mL) were added, the organic layer was washed with brine, and dried over MgSO₄. After the solvent was removed in vacuo, the residue was purified by silica gel column chromatography with 7:1 Hex/EtOAc to give the title compound (212 mg, 83%). ¹H NMR (400 MHz, CDCl₃): δ, 1.34 (s, 18H), 6.49 (d, J=9.9 Hz, 1H), 6.77 (s, 1H), 7.42 (d, J=9.0 Hz, 1H), 7.64 (d, J=9.8 Hz, 1H), 8.12 (d, J=9.0 Hz, 1H). ¹³C NMR (100 MHz, CDCl₃): δ, 27.8, 33.6, 84.6, 115.4, 118.1, 118.2, 128.1, 129.8, 133.0, 135.4, 137.3, 149.3, 154.2, 158.9. IR υₘₐₓ(NaCl)/cm⁻¹: 1795, 1747, 1272, 1250, 1103, 847. MS (APCI⁺) calculated for C₂₀H₂₃Br₂NO₆m/z 533.21, observed m/z 534.0.

Compound 99

To a solution of compound 98 (150 mg, 0.28 mmol) in acetone (20 mL) and H₂O (10 mL) at RT was AgNO₃ (317 mg, 1.69 mmol) added. The reaction mixture was stirred at 65° C. for 20 min. The volatile solvent was removed in vacuo. EtOAc (30 mL) and H₂O (10 mL) were added, the organic layer was washed with brine, and dried over MgSO₄. After the solvent was removed in vacuo, the residue was purified by silica gel column chromatography with 4:1 Hex/EtOAc to give the title compound (99 mg, 91%). ¹H NMR (400 MHz, CDCl₃): δ, 1.40 (s, 18H), 6.56 (d, J=9.8 Hz, 1H), 7.45 (d, J=8.6 Hz, 1H), 7.79 (d, J=9.8 Hz, 1H), 8.07 (d, J=8.7 Hz, 1H), 10.1 (s, 1H). IR υₘₐₓ(NaCl)/cm⁻¹: 2979, 1747, 1596, 1371, 1279, 1165, 1119, 890. MS (APCI⁺) calculated for C₂₀H₂₃NO₇ m/z 389.40, observed m/z 390.3.

Compound 102

Prepared from compound 97 in a same manner as compound 98 (1.0 eq of NBS was used here). ¹H NMR (400 MHz, CDCl₃): δ, 1.39 (s, 18H), 4.43 (s, 2H), 6.49 (d, J=9.8 Hz, 1H), 7.33 (d, J=8.7 Hz, 1H), 7.65 (m, 2H). ¹³C NMR (100 MHz, CDCl₃): δ, 27.3, 28.0, 84.2, 117.1, 117.3, 117.8, 131.4, 133.0, 133.4, 134.6, 137.8, 150.0, 153.9, 159.3. IR υₘₐₓ(NaCl)/cm⁻¹: 1740, 1701, 1358, 1272, 1240, 1158, 1125, 838. MS (APCI⁺) calculated for C₂₀H₂₄BrNO₆ m/z 454.31, observed m/z 455.2.

Compound 103

To a solution of compound 102 (257 mg, 0.56 mmol) in dry DMSO (8 mL) was KCN (40 mg, 0.62 mmol) added. The reaction mixture was stirred at RT for 2 h. EtOAc (30 mL) and H₂O (30 mL) were added; the organic layer was washed with H₂O (3×30 mL), brine, and dried over MgSO₄. After the solvent was removed in vacuo, the residue was purified by silica gel column chromatography with 4:1 Hex/EtOAc to give the title compound (146 mg, 65%). ¹H NMR (400 MHz, CDCl₃): δ, 1.39 (s, 18H), 3.68 (s, 2H), 6.25 (d, J=9.8 Hz, 1H), 7.38 (d, J=8.7 Hz, 1H), 7.67 (m, 2H). ¹³C NMR (100 MHz, CDCl₃): δ, 19.2, 27.9, 84.7, 116.1, 117.3, 117.4, 118.3, 124.7, 131.3, 134.5, 137.3, 149.8, 153.7, 159.0. IR υₘₐₓ(NaCl)/cm⁻¹: 1793, 1740, 1272, 1251, 1150, 1102. MS (APCI⁺) calculated for C₂₁H₂₄N₂O₆m/z 400.43, observed m/z 401.3.

Compound 105

Prepared from 6-methylcoumarin in a same manner as compound 102. 1H NMR (400 MHz, CDCl₃): δ, 4.51 (s, 2H), 6.42 (d, J=9.6 Hz, 1H), 7.28 (d, J=8.5 Hz, 1H), 7.50 (d, J=2.0 Hz, 1H), 7.54 (dd, J=2.1, 8.5 Hz, 1H), 7.67 (d, J=9.5 Hz, 1H). ¹³C NMR (100 MHz, CDCl₃): δ, 32.1, 117.0, 117.2, 118.7, 128.0, 132.3, 134.0, 142.7, 153.4, 160.0. IR υₘₐₓ(NaCl)/cm⁻¹: 1716, 1616, 1575, 1168, 909, 832. MS (APCI⁺) calculated for C₁₀H₇BrO₂ m/z 239.07, observed m/z 239.9.

Compound 106

A solution of compound 105 (239 mg, 1 mmol) and Pd(PPh₃)₄ (57 mg, 0.05 mmol) in THF (15 mL) was purged with CO and kept at 50° C. under one atmosphere of CO. Bu₃SnH (296 μL, 1.1 mmol, dissolved in 10 mL of THF) was added to the reaction mixture over 2 h using syringe pump. The reaction was quenched with H₂O and EtOAc (50 mL) and H₂O (50 mL) added. The organic layer was washed with brine, dried over anhydrous MgSO₄, and concentrated in vacuo. The residue was purified by silica gel column chromatography with 3:1 Hex/EtOAc to give the title compound (<20%). ¹H NMR (400 MHz, CDCl₃): δ, 3.78 (d, J=1.9 Hz, 2H), 6.42 (d, J=9.6 Hz, 1H), 7.34 (m, 3H), 7.67 (d, J=9.6 Hz, 1H), 9.79 (s, 1H). ¹³C NMR (100 MHz, CDCl₃): δ, 49.5, 117.0, 117.2, 118.9, 128.0, 128.6, 132.8, 142.8, 153.0, 160.2, 197.8. IR υₘₐₓ(NaCl)/cm⁻¹: 1722, 1574, 1262, 1170, 1102, 926, 841. MS (APCI⁺) calculated for C₁₁H₈O₃ m/z 188.18, observed m/z 189.1.

Compound 107

Prepared from compound 105 in a same manner as compound 103. ¹H NMR (400 MHz, CDCl₃): δ, 3.82 (s, 2H), 6.45 (d, J=9.5 Hz, 1H), 7.33 (d, J=8.4 Hz, 1H), 7.46 (dd, J=2.0, 8.5 Hz, 1H), 7.49 (s, 1H), 7.69 (d, J=9.5 Hz, 1H). ¹³C NMR (100 MHz, CDCl₃): δ, 23.1, 117.0, 117.5, 117.6, 119.0, 126.1, 126.8, 131.0, 142.4, 153.3, 159.8. IR υₘₐₓ(NaCl)/cm⁻¹: 1724, 1572, 1146, 1103, 915, 820. MS (APCI⁺) calculated for C₁₁H₈O₃ m/z 185.18, observed m/z 186.2.

Compound 108

A suspension of compound 107 (1.40 g, 7.56 mmol) in AcOH (8 mL), conc. H₂SO₄ (8 mL), and H₂O (8 mL) was stirred at 105° C. for 3 h. A lot of white precipitate formed when cooled down to RT. The white solid was collected, washed wish H₂O, and dried over P₂O₅ to give the title compound as a white solid (1.46 g, 94%). ¹H NMR (400 MHz, DMSO-d₆): δ, 3.65 (s, 2H), 6.47 (d, J=9.5 Hz, 1H), 7.34 (d, J=8.5 Hz, 1H), 7.50 (dd, J=2.0, 8.5 Hz, 1H), 7.59 (d, J=2.0 Hz, 1H), 8.04 (d, J=9.5 Hz, 1H), 12.4 (bs, 1H). ¹³C NMR (100 MHz, DMSO-d₆): δ, 40.1, 115.8, 116.0, 118.2, 128.6, 131.1, 132.9, 143.8, 152.0, 159.6, 172.0. IR υₘₐₓ(NaCl)/cm⁻¹: 3085, 1731, 1686, 1573, 1264, 1203, 837. MS (APCI⁺) calculated for C₁₁H₈O₄ m/z 204.18, observed m/z 205.1.

Compound 109

To a solution of compound 86 (34 mg, 0.18 mmol) and conc. H₂SO₄ (8.9 μL, 0.18 mmol) in Ac₂O (3 mL) was fuming HNO₃ (8.9 μL, 0.20 mmol) added at 0° C. The reaction mixture was stirred at that temperature for 2 h. EtOAc (20 mL) and H₂O (20 mL) were added; the organic layer was washed with H₂O (3×30 mL), brine, and dried over MgSO₄. After the solvent was removed in vacuo, the residue was purified by silica gel column chromatography with 1:1 Hex/EtOAc to give the title compound (20 mg, 40%). ¹H NMR (300 MHz, CDCl₃): δ, 2.04 (s, 3H), 3.06 (t, J=6.6 Hz, 2H), 4.34 (t, J=6.6 Hz, 2H), 6.54 (d, J=9.6 Hz, 1H), 7.59 (d, J=2.0 Hz, 1H), 7.74 (d, J=9.6 Hz, 1H), 7.99 (d, J=2.0 Hz, 1H). ¹³C NMR (75 MHz, CDCl₃): δ, 20.8, 34.0, 63.6, 118.3, 120.7, 127.7, 132.7, 134.6, 137.4, 142.3, 145.3, 157.7, 170.7. IR υₘₐₓ(NaCl)/cm⁻¹: 1732, 1533, 1352, 1241, 1108, 1036. MS (APCI⁺) calculated for C₁₃H₁₁NO₆ m/z 277.23, observed m/z 278.1.

Compound 110

Prepared from compound 109 in a same manner as compound 8. ¹H NMR (400 MHz, CDCl₃): δ, 2.01 (s, 3H), 2.86 (t, J=7.0 Hz, 2H), 4.06 (bs, 2H), 4.24 (t, J=7.0 Hz, 2H), 6.36 (d, J=9.5 Hz, 1H), 6.67 (s, 1H), 6.76 (s, 1H), 7.60 (d, J=9.5 Hz, 1H). ¹³C NMR (100 MHz, CDCl₃): δ, 21.1, 34.6, 64.6, 116.1, 116.5, 117.5, 118.6, 134.2, 134.4, 140.2, 143.9, 160.3, 170.6. IR υₘₐₓ(NaCl)/cm⁻¹: 3441, 3357, 1712, 1252, 1033, 842. MS (APCI⁺) calculated for C₁₃H₁₃NO₄m/z 247.25, observed m/z 248.2.

Compound 111

Prepared from compound 106 in a same manner as compound 109. ¹H NMR (400 MHz, CDCl₃): δ, 2.07 (s, 6H), 3.19 (t, J=5.4 Hz, 2H), 6.55 (d, J=9.6 Hz, 1H), 6.92 (t, J=5.3 Hz, 1H), 7.64 (d, J=1.9 Hz, 1H), 7.76 (d, J=9.7 Hz, 1H), 8.02 (d, J=2.0 Hz, 1H). ¹³C NMR (100 MHz, CDCl₃): δ, 20.3, 20.8, 38.4, 89.0, 118.2, 120.5, 128.5, 130.5, 133.5, 137.0, 142.1, 145.4, 157.4, 168.4. IR υₘₐₓ(NaCl)/cm⁻¹: 3449, 1745, 1539, 1361, 1243, 1194, 1013. MS (APCI⁺) calculated for C₁₃H₁₃NO₄m/z 335.27, observed m/z 336.3.

Compound 114

Prepared from compound 123 in a same manner as compound 3. ¹H NMR (300 MHz, CD₃OD): δ, 1.21 (t, J=7.0 Hz, 6H), 2.23 (s, 3H), 2.96 (t, J=7.0 Hz, 2H), 3.23 (t, J=7.2 Hz, 2H), 3.49 (q, J=6.8 Hz, 4H), 6.55 (d, J=1.8 Hz, 1H), 6.78 (d, J=7.4 Hz, 1H), 6.98 (d, J=8.3 Hz, 1H), 7.05 (m, 2H), 7.58 (d, J=9.1 Hz, 1H). ¹³C NMR (75 MHz, CD₃OD): δ, 12.8, 16.7, 30.2, 39.7, 45.7, 97.9, 108.3, 110.5, 117.0, 118.8, 120.8, 123.6, 127.5, 128.5, 131.7, 133.4, 142.9, 151.3, 151.6, 155.8, 162.2, 164.7. IR υₘₐₓ(NaCl)/cm⁻¹: 3435, 1685, 1618, 1523, 1204, 1136. MS (APCI⁺) calculated for C₂₂H₂₇N₃O₂ m/z 365.47, observed m/z 366.3.

Compound 115

A solution of compound 124 (105 mg, 0.17 mmol) in TFA (5 mL) was refluxed under Ar for 1 h. The reaction mixture was cooled down to RT, quenched with 1 drop of triethylsilane, and concentrated in vacuo. The residue was purified by silica gel flash chromatography with 10:1 CH₂Cl₂/MeOH to give the title compound (26 mg, ˜70%). ¹H NMR (400 MHz, CD₃OD): δ, 2.73 (s, 3H), 3.12 (t, J=8.0 Hz, 2H), 3.30 (t, J=8.0 Hz, 2H), 6.31 (s, 1H), 7.10 (d, J=8.9 Hz, 1H), 7.14 (d, J=8.9 Hz, 1H). IR υₘₐₓ(NaCl)/cm⁻¹: 3377, 1680, 1203, 1124, 722. HRMS (FAB⁺) calculated for C₁₂H₁₅N₂O₂ m/z 219.1134, observed m/z 219.1142.

Compound 116

Prepared from compound 127 in a same manner as compound 115. ¹H NMR (300 MHz, CD₃OD): δ, 2.97 (t, J=7.3 Hz, 2H), 3.22 (t, J=7.3 Hz, 2H), 6.33 (d, J=9.5 Hz, 1H), 6.93 (s, 1H), 7.09 (s, 1H), 7.79 (d, J=9.5 Hz, 1H). ¹³C NMR (75 MHz, CD₃OD): δ, 30.6, 39.2, 114.0, 116.9, 118.6, 120.2, 127.9, 144.6, 145.4, 148.1, 163.5. IR υₘₐₓ(NaCl)/cm⁻¹: 3443, 1701, 1560, 1434, 1210, 1180, 1137. MS (APCI⁺) calculated for C₁₁H₁₂N₂O₂ m/z 204.23, observed m/z 205.2.

Compound 117

A suspension of compound 5 (1.23 g, 3.90 mmol) and Raney Ni (cat) in EtOH (20 mL) was hydrogenated under 50 psi of H₂ at RT for 2 h. The catalyst was filtered off and the solvent removed in vacuo. The residue was purified by silica gel flash chromatography with Hex/EtOAc (2/1) to give the title compound (1.00 g, 90%). ¹H NMR (300 MHz, CDCl₃): δ, 1.43 (s, 9H), 3.40 (s, 2H), 4.0 (bs, 2H), 6.54 (d, J=8.4 Hz, 1H), 7.14 (dd, J=2.0, 8.4 Hz, 1H), 7.17 (d, J=1.8 Hz, 1H). ¹³C NMR (75 MHz, CDCl₃): δ, 28.0, 39.6, 81.5, 109.9, 117.7, 121.8, 130.6, 133.1, 144.4, 170.2. IR υₘₐₓ(NaCl)/cm⁻¹: 3409, 1704, 1490, 1334, 1242, 1153. MS (APCI⁺) calculated for C₁₂H₁₆BrNO₄ m/z 286.16, observed m/z 287.1.

Compound 118

Prepared from compound 117 in a same manner as compound 9. ¹H NMR (400 MHz, CDCl₃): δ, 1.44 (s, 9H), 1.51 (s, 9H), 3.46 (s, 2H), 7.30 (d, J=2.3 Hz, 1H), 7.14 (dd, J=2.3, 8.7 Hz, 1H), 7.68 (bs, 1H), 7.71 (d, J=8.6 Hz, 1H). ¹³C NMR (100 MHz, CDCl₃): δ, 28.0, 28.4, 40.0, 80.5, 82.5, 116.5, 124.0, 126.9, 130.8, 133.0, 136.2, 152.9, 170.2. IR υₘₐₓ(NaCl)/cm⁻¹: 3335, 1722, 1510, 1330, 1257, 1142. MS (APCI⁺) calculated for C₁₇H₂₄BrNO₄ m/z 386.28, observed m/z 387.2.

Compound 119

To a solution of compound 118 (386 mg, 1 mmol) in THF (10 mL) was DIBAL-H (3 mL, 3 mmol, 1 M in Hex) added at −78° C. under Ar for 1 h. The reaction mixture was stirred at that temperature for 1 h and quenched with H₂O (20 mL). EtOAc (20 mL) was added and the suspension was filtered through celite. The organic layer was washed with brine, dried over MgSO₄, and concentrated in vacuo. The residue was purified by silica gel flash chromatography with Hex/EtOAc (4/1) to give the title compound (171 mg, 54%). ¹H NMR (400 MHz, CDCl₃): δ, 1.50 (s, 9H), 2.16 (bs, 1H), 2.78 (t, J=5.5 Hz, 2H), 3.89 (t, J=5.7 Hz, 2H), 7.26 (d, J=2.1 Hz, 1H), 7.32 (dd, J=2.3, 8.7 Hz, 1H), 7.62 (d, J=8.1 Hz, 1H), 7.74 (bs, 1H). ¹³C NMR (100 MHz, CDCl₃): δ, 28.5, 34.5, 64.1, 80.4, 116.5, 124.2, 129.9, 132.6, 132.9, 136.3, 153.3. IR υₘₐₓ(NaCl)/cm⁻¹: 3513, 3360, 1705, 1504, 1243, 1162, 1051. MS (APCI⁺) calculated for C₁₃H₁₈BrNO₃ m/z 316.19, observed m/z 317.2.

Compound 120

Et₃N (233 μL, 1.68 mmol) was added dropwise to a solution of compound 119 (443 mg, 1.40 mmol) and TsCl (320 mg, 1.68 mmol) in CH₂Cl₂ (15 mL) with stirring at 0° C. After stirring at RT for 10 h, water (50 mL) and EtOAc (50 mL) was added, and the organic layer was separated, washed with water and brine, dried over MgSO₄, and concentrated in vacuo. The crude intermediate was dissolved in DMSO (10 mL) and NaN₃ (182 mg, 2.80 mmol) added. After stirring at 80° C. for 10 h, water (50 mL) and EtOAc (50 mL) was added, and the organic layer was separated, washed with H₂O (2×30 mL) and brine, dried over MgSO₄, and concentrated in vacuo. The residue was purified by silica gel column chromatography with 10:1 Hex/EtOAc to give the title compound (337 mg, 70%). ¹H NMR (400 MHz, CDCl₃): δ, 1.52 (s, 9H), 2.77 (t, J=6.5 Hz, 2H), 3.62 (t, J=6.5 Hz, 2H), 6.77 (bs, 1H), 7.29 (d, J=2.2 Hz, 1H), 7.35 (dd, J=2.3, 8.6 Hz, 1H), 7.58 (d, J=8.5 Hz, 1H). ¹³C NMR (100 MHz, CDCl₃): δ, 28.5, 31.1, 52.4, 64.1, 80.8, 117.3, 125.1, 130.5, 131.8, 132.4, 135.4, 153.0. IR υₘₐₓ(NaCl)/cm⁻¹: 3344, 2018, 1688, 1511, 1251, 1158. MS (APCI⁺) calculated for C₁₃H₁₇BrN₄O₂ m/z 341.20, observed m/z 342.1.

Compound 121

Compound 120 (290 mg, 0.85 mmol) and Boc₂O (278 mg, 1.27 mmol) in EtOAc (40 mL) was hydrogenated over PtO₂ at RT under 1 atmospheric pressure of H₂ for 8 h. The reaction mixture was concentrated in vacuo. The residue was purified by silica gel column chromatography with 8:1 Hex/EtOAc to give the title compound (322 mg, 91%). ¹H NMR (400 MHz, CDCl₃): δ, 1.46 (s, 9H), 1.52 (s, 9H), 2.74 (t, J=7.1 Hz, 2H), 3.18 (m, 2H), 4.91 (bs, 1H), 7.19 (d, J=1.9 Hz, 1H), 7.30 (dd, J=2.3, 8.8 Hz, 1H), 7.77 (s, 1H), 7.82 (d, J=8.2 Hz, 1H). ¹³C NMR (100 MHz, CDCl₃): δ, 28.5, 32.0, 41.0, 80.0, 80.2, 115.6, 123.0, 130.0, 132.1, 136.0, 153.3, 156.5, 161.8. IR υₘₐₓ(NaCl)/cm⁻¹: 3306, 1692, 1526, 1245, 1162. MS (APCI⁺) calculated for C₁₈H₂₇BrN₂O₄ m/z 415.32, observed m/z 416.2.

Compound 122

Prepared from compound 121 in a same manner as compound 19.

Compound 123

Prepared from compounds 18 and 122 in a same manner as compound 12. ¹H NMR (400 MHz, CDCl₃): δ, 1.21 (t, J=7.0 Hz, 6H), 1.46 (s, 9H), 1.54 (s, 9H), 2.22 (s, 3H), 2.81 (t, J=7.3 Hz, 2H), 3.28 (m, 2H), 3.42 (q, J=7.0 Hz, 4H), 6.54 (s, 1H), 6.62 (d, J=8.2 Hz, 1H), 7.06 (s, 1H), 7.14 (d, J=8.2 Hz, 2H), 7.43 (d, J=9.0 Hz, 1H), 7.52 (bs, 1H), 7.93 (d, J=7.8 Hz, 1H). ¹³C NMR (100 MHz, CDCl₃): δ, 12.6, 16.6, 25.0, 28.5, 32.2, 41.1, 44.9, 79.7, 80.1, 97.5, 108.5, 109.6, 120.5, 121.5, 125.9, 128.0, 129.4, 130.1, 131.6, 148.1, 149.8, 153.5, 154.7, 156.4, 161.8. IR υₘₐₓ(NaCl)/cm⁻¹: 3302, 1703, 1690, 1619, 1586, 1271, 1164. MS (APCI⁺) calculated for C₃₂H₄₃N₃O₆ m/z 565.70, observed m/z 566.6.

Compound 124

A mixture of compound 70 (200 mg, 0.85 mmol), NBS—NH₂ (268 mg, 1.28 mmol), and NaBH(OAc)₃ (362 mg, 1.7 mmol) in ClCH₂CH₂Cl (20 mL) was stirred under Ar at RT for 6 hrs. EtOAc (50 mL) and H₂O (50 mL) were added. The organic layer was washed successively with water and brine, dried over anhydrous MgSO₄, and concentrated in vacuo. The residue was purified by silica gel flash chromatography with 4:1 Hex/EtOAc to give the title compound (452 mg, 87%). ¹H NMR (400 MHz, CDCl₃): δ, 1.64 (s, 18H), 2.45 (s, 3H), 2.64 (t, J=7.7 Hz, 2H), 2.89 (m, 2H), 3.05 (t, J=8.3 Hz, 2H), 3.77 (m, 2H), 4.66 (s, 1H), 6.18 (s, 1H), 7.13 (m, 8H), 7.21 (s, 1H), 7.26 (s, 1H). ¹³C NMR (100 MHz, CDCl₃): δ, 24.4, 28.0, 30.0, 32.5, 49.4, 70.6, 83.2, 116.6, 117.7, 119.3, 125.7, 127.5, 129.3, 130.2, 131.5, 135.2, 136.5, 139.3, 139.8, 151.3, 153.0, 154.0, 159.4. IR υₘₐₓ(NaCl)/cm⁻¹: 1791, 1731, 1546, 1368, 1273, 1153, 1114. MS (APCI⁺) calculated for C₃₇H₄₂N₂O₆ m/z 610.74, observed m/z 611.6.

Compound 125

¹H NMR (300 MHz, CDCl₃): δ, 1.43 (s, 3H), 2.47 (m, 2H), 2.57 (d, J=15.3 Hz, 1H), 2.81 (d, J=15.3 Hz, 1H), 3.36 (m, 2H), 3.48 (bs, 2H), 6.57 (d, J=8.5 Hz, 1H), 6.78 (d, J=8.5 Hz, 1H). ¹³C NMR (100 MHz, CDCl₃): δ, 23.3, 24.7, 37.7, 44.5, 49.6, 113.9, 114.6, 117.7, 126.8, 141.0, 168.3. IR υₘₐₓ(NaCl)/cm⁻¹: 3420, 1752, 1658, 1481, 1225. MS (APCI⁺) calculated for C₁₂H₁₄N₂O₂ m/z 218.25, observed m/z 219.1.

Compound 126

Prepared from compound 79 in a same manner as compound 124. ¹H NMR (300 MHz, CDCl₃): δ, 2.88 (m, 2H), 2.93 (t, J=6.5 Hz, 2H), 3.12 (t, J=6.5 Hz, 2H), 3.63 (m, 2H), 6.18 (d, J=9.5 Hz, 1H), 7.11 (m, 10H), 7.67 (d, J=9.5 Hz, 1H), 8.08 (s, 1H). ¹³C NMR (75 MHz, CDCl₃): δ, 32.6, 34.4, 48.2, 69.8, 117.0, 118.1, 120.2, 124.8, 125.9, 127.6, 129.2, 130.5, 136.7, 139.7, 140.0, 140.3, 141.7, 145.3, 155.2, 158.8. IR υₘₐₓ(NaCl)/cm⁻¹: 1745, 1626, 1528, 1327, 1102. MS (APCI⁺) calculated for C₂₆H₂₂N₂Q₄ m/z 426.46, observed m/z 427.3.

Compound 127

A suspension of compound 126 (223 mg, 0.52 mmol) and SnCl₂.2H₂O (588 mg, 2.6 mmol) in EtOH (25 mL) was refluxed under Ar for 2 hrs. The reaction mixture was then concentrated in vacuo; the residue was poured into 50 mL of EtOAc and 50 mL of water and filtered through celite pad. The organic layer was separated and washed successively with water and brine, dried over anhydrous MgSO₄, and concentrated in vacuo. The residue was purified by silica gel flash chromatography with 2:1 Hex/EtOAc to give the title compound (136 mg, 66%). ¹H NMR (400 MHz, CDCl₃): δ, 2.70 (t, J=6.5 Hz, 2H), 2.88 (m, 2H), 2.96 (m, 2H), 3.64 (m, 2H), 6.30 (d, J=9.5 Hz, 1H), 6.63 (s, 1H), 6.96 (s, 1H), 7.13 (m, 6H), 7.25 (m, 2H), 7.51 (d, J=9.5 Hz, 1H). ¹³C NMR (75 MHz, CDCl₃): δ, 32.5, 32.7, 47.5, 66.4, 111.8, 115.8, 117.5, 125.8, 127.4, 128.6, 130.3, 130.9, 139.5, 141.6, 142.7, 147.0, 161.1. IR υₘₐₓ(NaCl)/cm⁻¹: 3353, 1748, 1634, 1529, 1105. MS (APCI⁺) calculated for C₂₆H₂₄N₂O₂ m/z 396.48, observed m/z 397.3.

Syntheses of Probes 9 and 10

The following are the syntheses and structural characterization of probes 9 and 10:

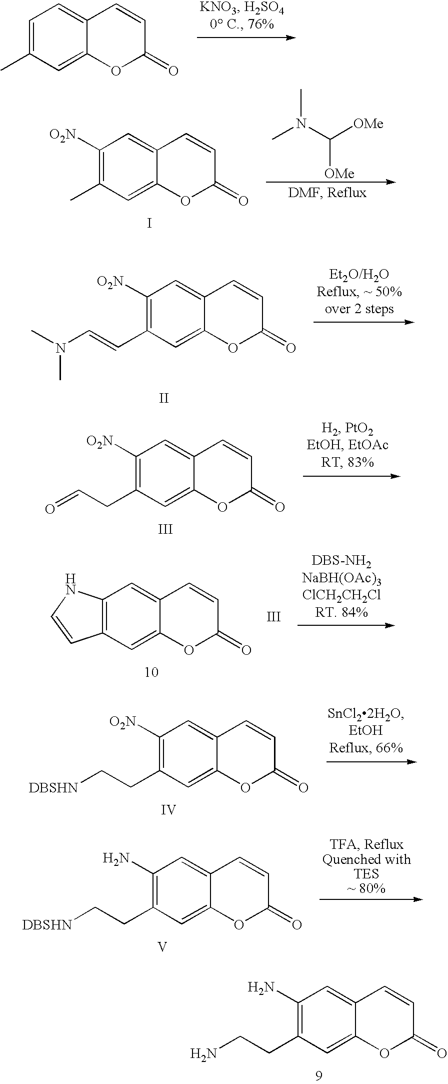

Compound III

410 mg (2.0 mmol) of compound I (known in the art Lin, S. T., Yang, F. M. m Yang, H. Y., Huang, K. F. J. Chem. Research (S) 1995, 372-373.) and 0.40 mL (3.0 mmol) of N,N-Dimethylformamide dimethyl acetal was refluxed under Ar in 10 mL of anhydrous DMF for 1 hr. The reaction mixture was concentrated in vacuo to get crude product II. 20 mL of Et₂O and 20 mL of 3 N aq. HCl was added and the mixture was refluxed for 1 hr. 50 mL of Et₂O and 50 mL of water were then added. The organic layer was washed with brine, dried over anhydrous MgSO₄, and concentrated in vacuo. The residue was purified by silica gel flash chromatography with 2:1 Hex/EtOAc to give the title compound (0.35 g, 52%). ¹H NMR (300 MHz, DMSO-d₆): δ, 4.33 (s, 2H), 6.66 (d, J=9.6 Hz, 1H), 7.56 (s, ¹H), 8.18 (d, J=9.6 Hz, ¹H), 8.62 (s, 1H), 9.75 (s, ¹H). ¹³C NMR (75 MHz, DMSO-d₆): δ, 47.7, 118.0, 118.2, 121.1, 125.6, 133.1, 142.9, 144.6, 155.7, 158.9, 198.2. IR υₘₐₓ(NaCl)/cm-1: 1739, 1623, 1530, 1326, 1269, 1110. MS (APCI+) calculated for C₁₁H₇NO₅ m/z 233.18, observed m/z 234.2.

Compound IV.

A mixture of 200 mg (0.85 mmol) of compound III, 268 mg (1.28 mmol) of NBS—NH₂, 362 mg (1.7 mmol) of NaBH(OAc)₃ in 20 mL of ClCH₂CH₂Cl was stirred under Ar at RT for 6 hr. The reaction mixture was then poured into 50 mL of EtOAc and 50 mL of water. The organic layer was washed successively with water and brine, dried over anhydrous MgSO₄, and concentrated in vacuo. The residue was purified by silica gel flash chromatography with 4:1 Hexane/EtOAc to give the title compound (315 mg, 84%). ¹H NMR (300 MHz, CdCl₃): δ, 2.88 (m, 2H), 2.93 (t, J=6.5 Hz, 2H), 3.12 (t, J=6.5 Hz, 2H), 3.63 (m, 2H), 6.18 (d, J=9.5 Hz, 1H), 7.11 (m, 10H), 7.67 (d, J=9.5 Hz, ¹H), 8.08 (s, ¹H). ¹³C NMR (75 MHz, CdCl₃): δ, 32.6, 34.4, 48.2, 69.8, 117.0, 118.1, 120.2, 124.8, 125.9, 127.6, 129.2, 130.5, 136.7, 139.7, 140.0, 140.3, 141.7, 145.3, 155.2, 158.8. IR υmax(NaCl)/cm-1: 1745, 1626, 1528, 1327, 1102. MS (APCI+) calculated for C₂₆H₂₂N₂O₄ m/z 426.46, observed m/z 427.3.

Compound V.

A suspension of 223 mg (0.52 mmol) of compound IV and 588 mg (2.6 mmol) of SnCl₂.2H₂O in 25 mL of EtOH was refluxed under Ar for 2 hr. The reaction mixture was then concentrated in vacuo; the residue was poured into 50 mL of EtOAc and 50 mL of water and filtered through a pad of celite. The organic layer was separated and washed successively with water and brine, dried over anhydrous MgSO₄, and concentrated in vacuo. The residue was purified by silica gel flash chromatography with 2:1 Hexane/EtOAc to give the title compound (136 mg, 66%). ¹H NMR (400 MHz, CdCl₃): δ, 2.70 (t, J=6.5 Hz, 2H), 2.88 (m, ²H), 2.96 (m, 2H), 3.64 (m, 2H), 6.30 (d, J=9.5 Hz, ¹H), 6.63 (s, 1H), 6.96 (s, 1H), 7.13 (m, 6H), 7.25 (m, ²H), 7.51 (d, J=9.5 Hz, 1H). ¹³C NMR (75 MHz, CDCl₃): δ, 32.5, 32.7, 47.5, 66.4, 111.8, 115.8, 117.5, 125.8, 127.4, 128.6, 130.3, 130.9, 139.5, 141.6, 142.7, 147.0, 161.1. IR υmax(NaCl)/cm-1: 3353, 1748, 1634, 1529, 1105. MS (APCI+) calculated for C₂₆H₂₄N₂O₂ m/z 396.48, observed m/z 397.3.

Probe 9.

105 mg (0.27 mmol) of compound V in 5 mL of TFA was refluxed under Ar for 1 hr. The reaction was quenched with 1 drop of triethylsilane at RT and then concentrated in vacuo. The residue was purified by silica gel flash chromatography with 10:1 CH₂Cl₂/MeOH to give the title compound (44 mg, 81%). ¹H NMR (300 MHz, CD3OD): δ, 2.97 (t, J=7.3 Hz, 2H), 3.22 (t, J=7.3 Hz, 2H), 6.33 (d, J=9.5 Hz, 1H), 6.93 (s, 1H), 7.09 (s, 1H), 7.79 (d, J=9.5 Hz, 1H). ¹³C NMR (75 MHz, CD3OD): δ, 30.6, 39.2, 114.0, 116.9, 118.6, 120.2, 127.9, 144.6, 145.4, 148.1, 163.5. IR υmax(NaCl)/cm-1: 3443, 1701, 1560, 1434, 1210, 1180, 1137. MS (APCI+) calculated for C₁₁H₁₂N₂O₂ m/z 204.23, observed m/z 205.2.

Probe 10.

122 mg (0.52 mmol) of compound III was dissolved in 10 mL of EtOH/EtOAc (1:1). The mixture was hydrogenated at 1 atm of H₂ using PtO₂ as the catalyst. The reaction solution was concentrated in vacuo. The residue was purified by silica gel flash chromatography with 10:1 CH₂Cl₂/MeOH to give the title compound (81 mg, 83%). ¹H NMR (300 MHz, CD3OD): δ, 6.36 (d, J=9.4 Hz, 1H), 6.61 (m, 1H), 7.58 (m, 2H), 7.70 (s, 1H), 8.09 (d, J=9.4 Hz, 1H). ¹³C NMR (75 MHz, CD3OD): δ, 22.5, 102.2, 106.0, 108.9, 112.2, 124.4, 125.1, 126.2, 129.7, 150.3, 152.5, 159.8. IR υmax(NaCl)/cm-1: 3276, 1688, 1630, 1508, 1313, 1142. MS (APCI+) calculated for C11H7NO2 m/z 185.18, observed m/z 186.1.

Photophysical Characterization

Extinction coefficients were the average of three independent determinations of three different readings from the lowest energy transition at three concentrations. Quantum yields are the average of three determinations of both quantum yield standard 9,10-diphenylanthracene and the synthesized coumarines at 340 nm unless otherwise indicated.

Enzymatic Activity Determinations

Activity of synthesized diamines with monoamine oxidase A and B were determined by 24 hour incubations with 10 micrograms of either human placenta mitochondria (MAO-A) or beef liver mitochondria (MAO-B) per milliliter of total assay volume. A twenty microliter aliquot of the respective diamine (prepared as a 2.5 millimolar stock concentration in dimethyl sulfoxide) were dissolved in 970 microliters of 100 mM sodium phosphate buffer (pH 7.4). Reactions were initiated with 10 microliters of a 30 mg/mL or 70 mg/mL mitochondrial protein mixture (as determined by standard Bradford assay) from human placenta and beef liver, respectively. Enzyme-catalyzed indole formation was realized after 24 hours by reading fluorescence emission upon excitation at the absorbance maxima of the respective indole. Product formation was confirmed by HPLC separation of the resultant assay mixture following centrifugation at 16,000 g, extraction of organic molecules by ethyl acetate, concentration, and resolvation before HPLC injection.

Derivation of Enzyme Kinetic Parameters

Enzymatic assays were performed as described above with the following modifications. 64 microliters of a range of stock preparations serially diluted 1 to 1 (20, 10, 5, 2.5, 1.25, 0.625, 0.3125, 0.15625, and 0.078125 mM in pH 5 phosphate buffer) were added to FALCON black microtiter 96-well plates. 136 microliters of 100 mM sodium phosphate buffer (pH 7.4) prepared with 10 microliters of dissolved MAO-A or MAO-B per milliliter of phosphate buffer was then added to the each well to produce final assay volumes of 200 microliters, with concentrations ranging from 400 micromolar to 1.5625 micromolar. Product formation from the resulting assay solutions were then assessed via fluorescence detection by a Micromax 386 connected to a Jobin-Yvon Fluorolog through fiber optic cables. Emission resulting from 335 nm excitation was determined every ten minutes for one hour. Enzymatic rates were calculated from the slope of the fluorescence growth curve and related to a standard curve of the fluorescence of the product.

TABLE 1
Index of Compounds for First Series of Experiments

| | | | ¹H- | ¹³C- |
|---|---|---|---|---|
| # | Name | Structure | NMR | NMR |
| 1 | 7-(4-Amino-phenyl)-4-methyl-chromen-2-one | 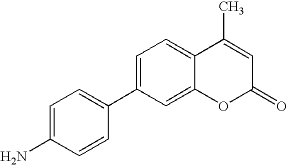 | NA(notavailable | NA |
| 2 | [4-(4-Methyl-2-oxo-2H-chromen-7-yl)-phenyl]-carbamic acid tert-butyl ester | 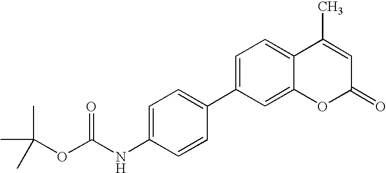 | NA | NA |
| 3 | 7-[4-Amino-3-(2-hydroxy-ethyl)-phenyl]-4-methyl-chromen-2-one | 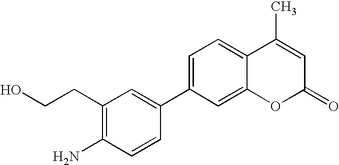 | GC-4-096300 MHzDMSO | s(same) |
| 4 | 7-(1H-Indol-5-yl)-4-methyl-chromen-2-one | 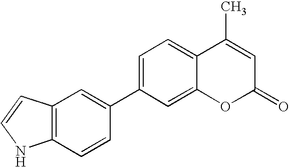 | GC-4-088300 MHzDMSO | s |
| 5 | (5-Bromo-2-nitro-phenyl)-aceticacid tert-butyl ester | 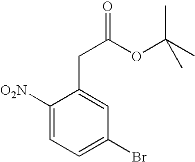 | GC-4-076300 MHzCDCl₃ | s |
| 6 | 2-(5-Bromo-2-nitro-phenyl)-ethanol | 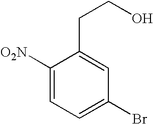 | GC-4-079400 MHzCDCl₃ | s |
| 7 | [2-(5-Bromo-2-nitro-phenyl)-ethoxy]-tert-butyl-dimethyl-silane | 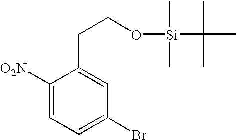 | GC-4-082400 MHzCDCl₃ | s |
| 8 | 4-Bromo-2-[2-(tert-butyl-dimethyl-silanyloxy)-ethyl]-phenylamine | 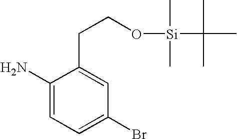 | GC-4-086400 MHzCDCl₃ | s |
| 9 | (4-Bromo-2-[2-(tert-butyl-dimethyl-silanyloxy)-ethyl]-phenyl)-carbamic acid tert-butylester | 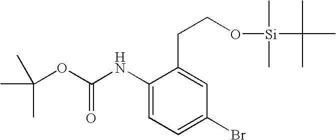 | GC-4-087400 MHzCDCl₃ | s |
| 10 | [2-[2-(tert-Butyl-dimethyl-silanyloxy)-ethyl]-4-(4,4,5,5-tetramethyl-1,3,2)dioxaborolan-2-yl]-phenyl]-carbamic acid tert-butyl ester | 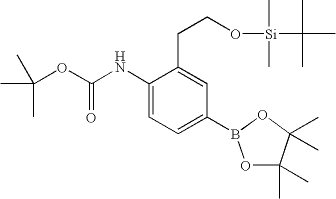 | NA | NA |
| 11 | Trifluoro-methanesulfonic acid 4-methyl-2-oxo-2H-chromen-7-ylester | 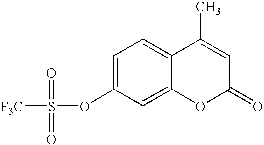 | GC-4-033400 MHzCDCl₃ | s |
| 12 | [2-[2-(tert-Butyl-dimethyl-silanyloxy)-ethyl]-4-(4-methyl-2-oxo-2H-chromen-7-yl)-phenyl]-carbamic acid tert-butyl ester | 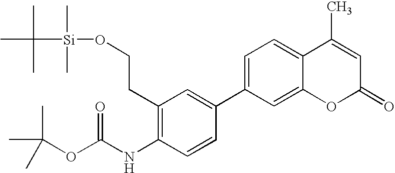 | GC-4-089300 MHzCDCl₃ | NA |
| 13 | [2-(2-Hydroxy-ethyl)-4-(4-methyl-2-oxo-2H-chromen-7-yl)-phenyl]-carbamic acid tert-butyl ester | 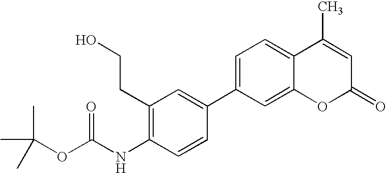 | GC-4-092400 MHzCDCl₃ | s |
| 14 | 2-Hydroxy-5-(4-methyl-2-oxo-2H-chromen-7-yl)-2,3-dihydro-indole-1-carboxylic acid tert-butylester | 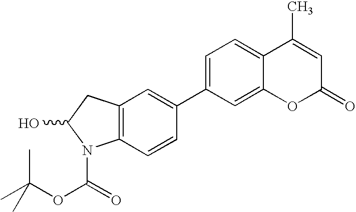 | GC-4-092 (3)400 MHzCDCl₃ | NA |
| 15 | 5-(4-Methyl-2-oxo-2H-chromen-7-yl)-indole-1-carboxylic acidtert-butyl ester | 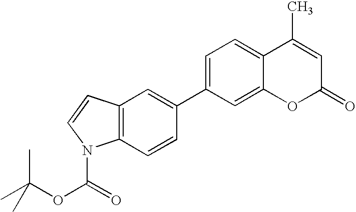 | GC-4-085400 MHzCDCl₃ | s |
| 16 | 3-[4-Amino-3-(2-hydroxy-ethyl)-phenyl]-7-diethylamino-4-methyl-chromen-2-one | 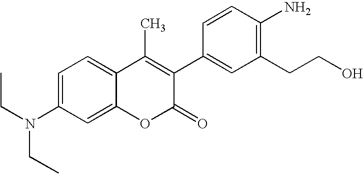 | GC-4-130300 MHzCDCl₃ | s |
| 17 | 7-Diethylamino-3-(1H-indol-5-yl)-4-methyl-chromen-2-one | 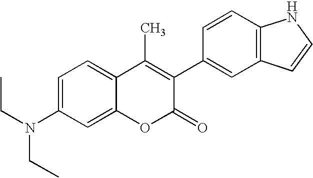 | GC-4-126400 MHzCDCl₃ | s |
| 18 | 3-Bromo-7-diethylamino-4-methyl-chromen-2-one | 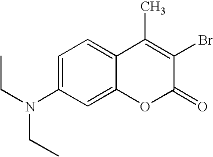 | GC-4-125400 MHzCDCl₃ | NA |
| 19 | 5-(4,4,5-Trimethyl-[1,3,2]dioxaborolan-2-yl)-indole-1-carboxylic acid tert-butylester | 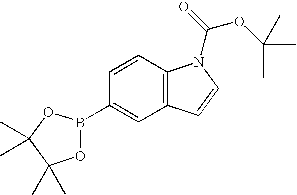 | NA | NA |
| 20 | 5-Bromo-indole-1-carboxylic acidtert-butyl ester | 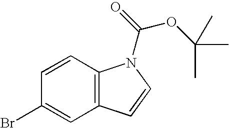 | GC-4-083300 MHzCDCl₃ | NA |
| 21 | 5-(7-Diethylamino-4-methyl-2-oxo-2H-chromen-3-yl)-indole-1-carboxylic acid tert-butyl ester | 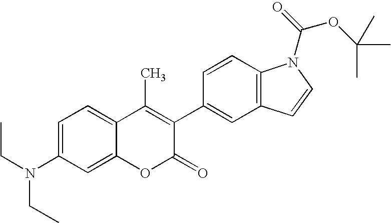 | NA | NA |
| 22 | [2-[2-(tert-Butyl-dimethyl-silanyloxy)-ethyl]-4-(7-diethylamino-4-methyl-2-oxo-2H-chromen-3-yl)-phenyl]-carbamic acid tert-butylester | 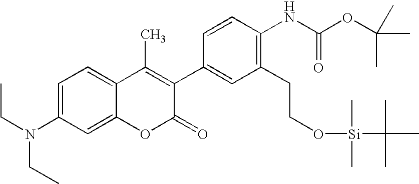 | GC-4-127300 MHzCDCl₃ | s |
| 23 | 9-[4-Amino-3-(2-hydroxy-ethyl)-phenyl]-8-methyl-2,3,5,6-tetrahydro-1H, 4H-11-oxa-3a-aza-benzo[de]anthracen-10-one | 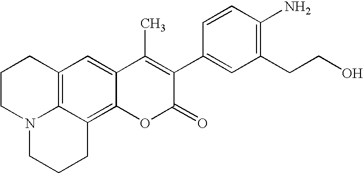 | GC-4-117300 MHzCDCl₃ | s |
| 24 | 9-(1H-Indol-5-yl)-8-methyl-2,3,5,6-tetrahydro-1H, 4H-11-oxa-3a-aza-benzo[de]anthracen-10-one | 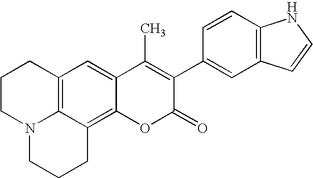 | GC-4-119 (5)400 MHzDMSO | s |
| 25 | 9-(1H-Indol-5-yl)-8-trifluoromethyl-2,3,5,6-tetrahydro-1H, 4H-11-oxa-3a-aza-benzo[de]anthracen-10-one | 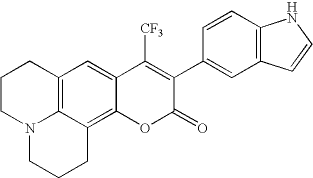 | GC-4-128300 MHzCDCl₃ | NA |
| 26 | 9-Bromo-8-methyl-2,3,5,6-tetrahydro-1H, 4H-11-oxa-3a-aza-benzo[de]anthracen-10-one | 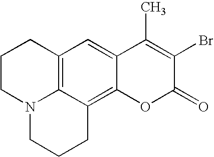 | GC-4-110400 MHzMeOH | NA |
| 27 | [2-[2-(tert-Butyl-dimethyl-silanyloxy)-ethyl]-4-(8-methyl-10-oxo-2,3,5,6-tetrahydro-1H, 4H, 10H-11-oxa-3a-aza-benzo[de]anthracen-9-yl)-phenyl]-carbamic acid tert-butyl ester | 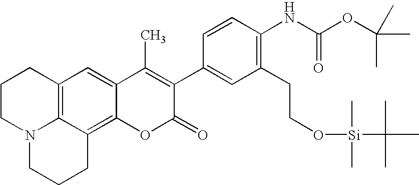 | GC-4-115400 MHzCDCl₃ | s |
| 28 | 7-Diethylamino-3-(1H-indol-6-yl)-4-methyl-chromen-2-one | 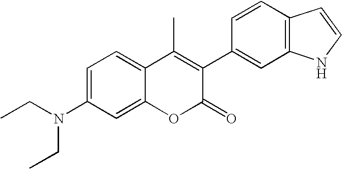 | GC-4-131300 MHzCDCl₃ | s |
| 29 | 3-[3-Amino-4-(2-hydroxy-ethyl)-phenyl]-7-diethylamino-4-methyl-chromen-2-one | 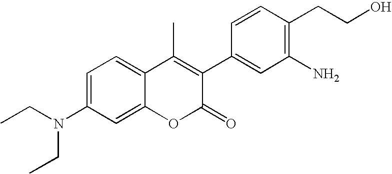 | NA | NA |
| 30 | 6-Bromo-indole-1-carboxylic acidtert-butyl ester | 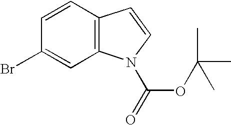 | GC-4-118300 MHzCDCl₃ | NA |
| 31 | 6-(4,4,5,5-Tetramethyl-[1,3,2]dioxaborolan-2-yl)-indole-1-carboxylic acid tert-butyl ester | 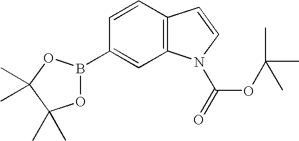 | NA | NA |
| 32 | 6-(7-Diethylamino-4-methyl-2-oxo-2H-chromen-3-yl)-indole-1-carboxylic acid tert-butyl ester | 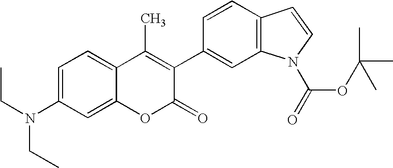 | GC-4-131 (3)400 MHzCDCl₃ | s |
| 33 | 6-Amino-5-(2-hydroxy-ethyl)-chromen-2-one | 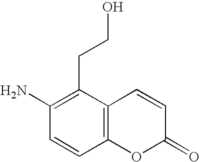 | GC-4-161400 MHzMeOH | s |
| 34 | 3H-Pyrano[3,2-e]indol-7-one | 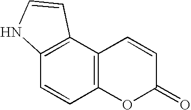 | GC-4-165400 MHzDMSO | s |
| 35 | 7-Amino-8-(2-hydroxy-ethyl)-4-methyl-chromen-2-one | 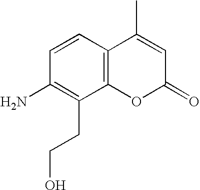 | NA | NA |
| 36 | 4-Methyl-7H-pyrano[2,3-e]indol-2-one | 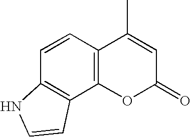 | GC-4-095300 MHzCDCl₃ | NA |
| 37 | 7-Allyloxy-4-methyl-chromen-2-one | 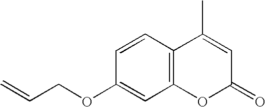 | GC-4-061400 MHzCDCl₃ | NA |
| 38 | 8-Allyl-7-hydroxy-4-methyl-chromen-2-one | 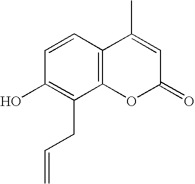 | GC-4-062300 MHzCDCl₃ | NA |
| 39 | Trifluoro-methanesulfonic acid 8-allyl-4-methyl-2-oxo-2H-chromen-7-yl ester | 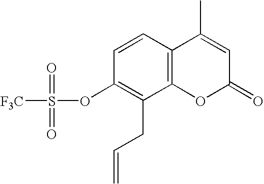 | GC-4-063400 MHzCDCl₃ | s |
| 40 | Trifluoro-methanesulfonic acid 4-methyl-2-oxo-8-(2-oxo-ethyl)-2H-chromen-7-yl ester | 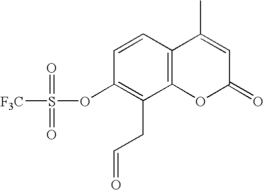 | GC-4-064400 MHzCDCl₃ | s |
| 41 | Trifluoro-methanesulfonic acid 8-(2-hydroxy-ethyl)-4-methyl-2-oxo-2H-chromen-7-yl ester | 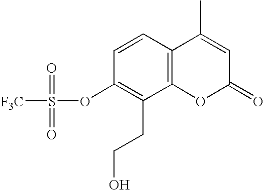 | GC-4-065400 MHzCDCl₃ | s |
| 42 | 4-Methyl-8,9-dihydro-furo[2,3-h]chromen-2-one | 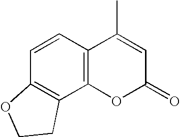 | GC-4-067300 MHzCDCl₃ | NA |
| 43 | Trifluoro-methanesulfonic acid 8-[2-(tert-butyl-dimethyl-silanyloxy)-ethyl]-4-methyl-2-oxo-2H-chromen-7-yl ester | 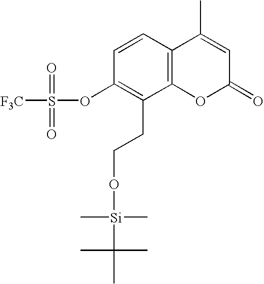 | GC-4-070400 MHzCDCl₃ | s |
| 44 | 7-(Benzhydrylidene-amino)-8-[2-(tert-butyl-dimethyl-silanyloxy)-ethyl]-4-methyl-chromen-2-one | 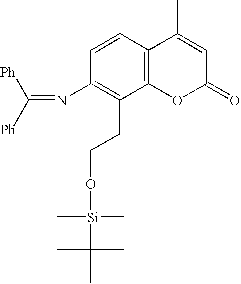 | NA | NA |
| 45 | 7-(1,1-Dimethyl-prop-2-ynylamino)-4-methyl-chromen-2-one | 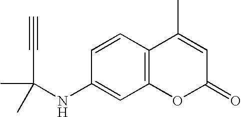 | GC-4-071400 MHzCDCl₃ | s |
| 46 | 7-(1,1-Dimethyl-allylamino)-4-methyl-chromen-2-one | 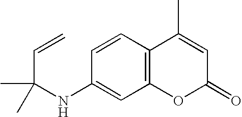 | GC-4-072400 MHzCDCl₃ | s |
| 47 | 7-Amino-4-methyl-8-(3-methyl-but-2-enyl)-chromen-2-one | 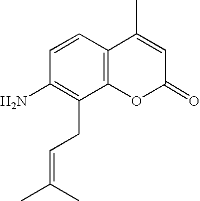 | GC-4-073400 MHzCDCl₃ | s |
| 48 | [4-Methyl-8-(3-methyl-but-2-enyl)-2-oxo-2H-chromen-7-yl]-carbamic acid 2,2,2-trichloro-ethyl ester | 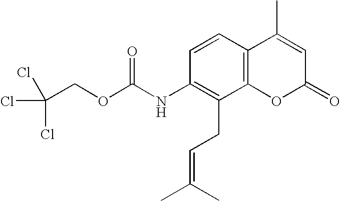 | GC-4-090400 MHzCDCl₃ | s |
| 49 | 8-Hydroxy-4-methyl-2-oxo-8,9-dihydro-2H-pyrano[2,3-e]indole-7-carboxylic acid 2,2,2-trichloro-ethyl ester | 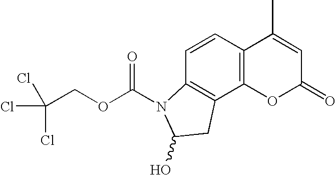 | GC-4-091400 MHzCDCl₃ | 300 MHz |
| 50 | 4-Methyl-2-oxo-2H-pyrano[2,3-e]indole-7-carboxylic acid 2,2,2-trichloro-ethyl ester | 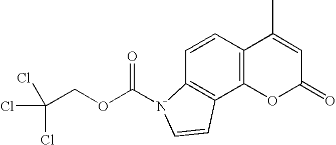 | NAMinor P | NA |
| 51 | But-2-ynoic acid 1H-indol-5-ylester | 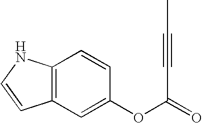 | GC-4-132400 MHzCDCl₃ | s |
| 52 | 9-Methyl-3H-pyrano[3,2-e]indol-7-one | 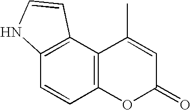 | GC-4-134400 MHzDMSO | s |
| 53 | 8-Methyl-1H-pyrano[2,3-f]indol-6-one | 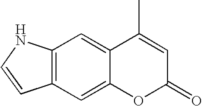 | NA | NA |
| 54 | But-2-ynoic acid 1H-indol-6-ylester | 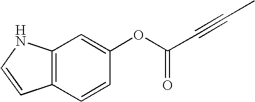 | GC-4-213400 MHzCDCl₃ | s |
| 55 | 9-Methyl-1H-pyrano[2,3-g]indol-7-one | 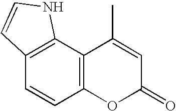 | GC-4-214400 MHzDMSO | s |
| 56 | 4-Methyl-8H-pyrano[3,2-f]indol-2-one | 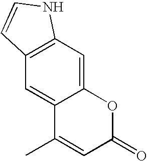 | NA | NA |
| 57 | 6-Amino-5-(2-hydroxy-ethyl)-4-methyl-chromen-2-one | 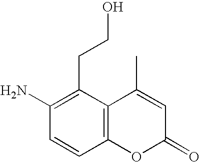 | GC-4-267400 MHzMeOH | s |
| 58 | 6-Amino-7-(2-hydroxy-ethyl)-chromen-2-one | 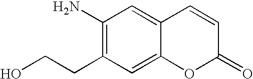 | NG-1-17300 MHzMeOH | s |
| 59 | 1H-Pyrano[2,3-f]indol-6-one | 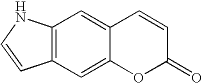 | NG-1-12400 MHzMeOH | NG-1-12400 MHzCDCl₃ |
| 60 | 4-Methyl-chromen-2-one | 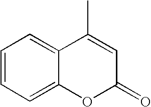 | GC-4-172400 MHzCDCl₃ | NA |
| 61 | 4-Methyl-6-nitro-chromen-2-one | 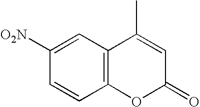 | GC-4-178400 MHzCDCl₃ | NA |
| 62 | 6-Amino-4-methyl-chromen-2-one | 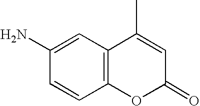 | GC-4-174400 MHzDMSO | s |
| 63 | 6-(1,1-Dimethyl-prop-2-ynylamino)-4-methyl-chromen-2-one | 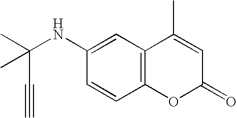 | GC-4-175400 MHzCDCl₃ | s |
| 64 | 6-(1,1-Dimethyl-allylamino)-4-methyl-chromen-2-one | 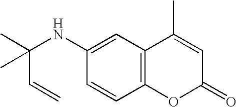 | GC-4-176400 MHzCDCl₃ | s |
| 65 | 6-Amino-4-methyl-5-(3-methyl-but-2-enyl)-chromen-2-one | 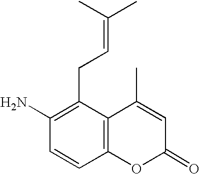 | GC-4-177400 MHzCDCl₃ | s |
| 66 | 2-[4-Methyl-5-(3-methyl-but-2-enyl)-2-oxo-2H-chromen-6-yl]-isoindole-1,3-dione | 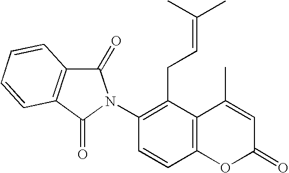 | GC-4-178400 MHzCDCl₃ | s |
| 67 | [6-(1,3-Dioxo-1,3-dihydro-isoindol-2-yl)-4-methyl-2-oxo-2H-chromen-5-yl)-acetaldehyde | 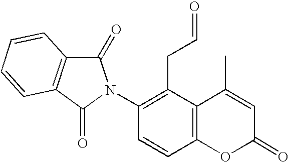 | GC-4-179400 MHzCDCl₃ | s |
| 68 | 2-[5-(2-Hydroxy-ethyl)-4-methyl-2-oxo-2H-chromen-6-yl]-isoindole-1,3-dione | 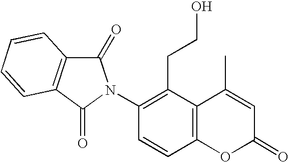 | GC-4-182300 MHzCDCl₃ | NA |
| 69 | [4-Methyl-5-(3-methyl-but-2-enyl)-2-oxo-2H-chromen-6-yl]-di-carbamic acid di-tert-butyl ester | 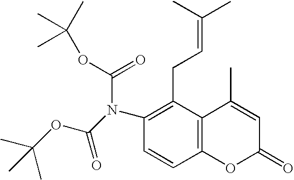 | GC-4-192400 MHzCDCl₃ | s |
| 70 | [4-Methyl-2-oxo-5-(2-oxo-ethyl)-2H-chromen-6-yl]-di-carbamic aciddi-tert-butyl ester | 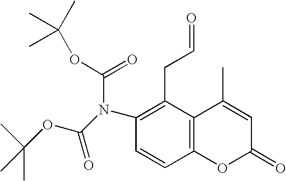 | GC-4-193400 MHzCDCl₃ | s |
| 71 | [5-(2-Hydroxy-ethyl)-4-methyl-2-oxo-2H-chromen-6-yl]-di-carbamicacid di-tert-butyl ester | 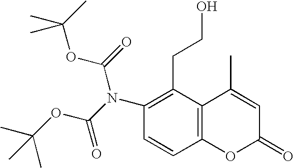 | GC-4-266300 MHzCDCl₃ | s |
| 72 | 7-Allyl-4-methyl-chromen-2-one | 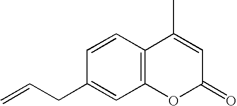 | GC-4-169400 MHzCDCl₃ | s |
| 73 | 1-Hydroxy-3,8-dimethyl-3,4-dihydro-1H-2,5-dioxo-1-aza-anthracen-6-one | 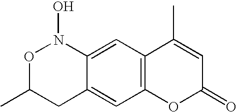 | GC-4-185300 MHzCDCl₃ | NA |
| 74 | (4-Methyl-2-oxo-2H-chromen-7-yl)-acetaldehyde | 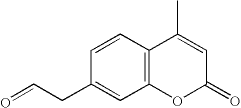 | GC-4-186400 MHzCDCl₃ | NA |
| 75 | 7-(2-Hydroxy-ethyl)-4-methyl-chromen-2-one | 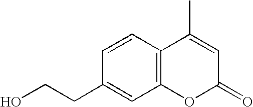 | GC-4-188300 MHzCDCl₃ | NA |
| 76 | Acetic acid 2-(6-nitro-2-oxo-2H-chromen-7-yl)-ethyl ester | 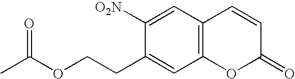 | NA | NA |
| 77 | 7-Methyl-6-nitro-chromen-2-one | 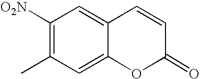 | NA | NA |
| 78 | 7-(2-Dimethylamino-vinyl)-6-nitro-chromen-2-one | 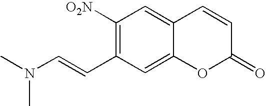 | NA | NA |
| 79 | (6-Nitro-2-oxo-2H-chromen-7-yl)-acetaldehyde | 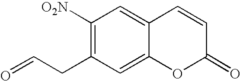 | NG-1-11300 MHzDMSO | s |
| 80 | 7-(2-Hydroxy-ethyl)-6-nitro-chromen-2-one | 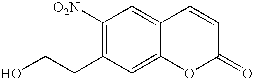 | NG-1-16300 MHzCDCl₃ | s |
| 81 | 5-Amino-6-(2-hydroxy-ethyl)-chromen-2-one | 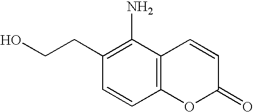 | GC-4-251300 MHzDMSO | s |
| 82 | 1H-Pyrano[2,3-g]indol-7-one | 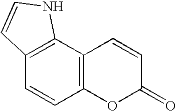 | NA | NA |
| 83 | 5-Amino-chromen-2-one | 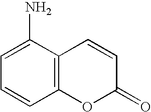 | NA | NA |
| 84 | Trifluoro-methanesulfonic acid 4-methyl-5-nitro-2-oxo-2H-chromen-6-yl ester | 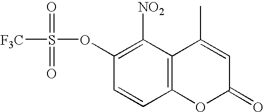 | GC-4-211400 MHzCDCl₃ | s |
| 85 | 6-Methyl-5-nitro-chromen-2-one | 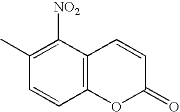 | NA | NA |
| 86 | 6-(2-Hydroxy-ethyl)-chromen-2-one | 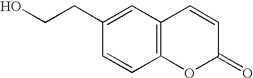 | GC-4-225400 MHzCDCl₃ | s |
| 87 | 5-Hydroxy-4-methyl-chromen-2-one | 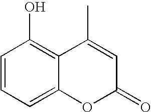 | GC-4-201400 MHzMeOH | NA |
| 88 | Trifluoro-methanesulfonic acid 4-methyl-2-oxo-2H-chromen-5-ylester | 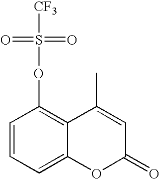 | GC-4-201300 MHzCDCl₃ | s |
| 89 | 6-Methoxy-4-methyl-5-nitro-chromen-2-one | 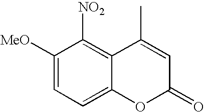 | NA | NA |
| 90 | 6-Hydroxy-4-methyl-5-nitro-chromen-2-one | 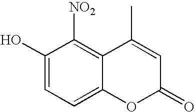 | GC-4-210400 MHzDMSO | s |
| 91 | 6-Allyl-4-methyl-5-nitro-chromen-2-one | 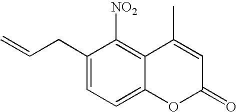 | NA | NA |
| 92 | 6-(2-Dimethylamino-vinyl)-5-nitro-chromen-2-one | 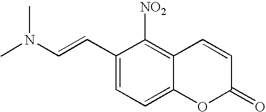 | NA | NA |
| 93 | (5-Nitro-2-oxo-2H-chromen-6-yl)-acetaldehyde | 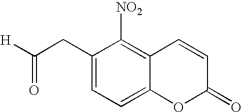 | GC-4-253300 MHzDMSO | s |
| 94 | 6-(2-Hydroxy-ethyl)-5-nitro-chromen-2-one | 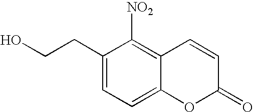 | GC-4-231400 MHzMeOH | s |
| 95 | 6-Bromomethyl-5-nitro-chromen-2-one | 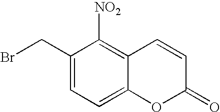 | NA | NA |
| 96 | 5-Amino-6-methyl-chromen-2-one | 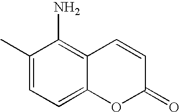 | NA | NA |
| 97 | (6-Methyl-2-oxo-2H-chromen-5-yl)-di-carbamic acid di-tert-butylester | 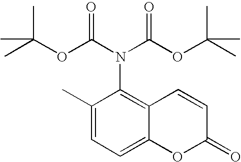 | GC-4-221400 MHzCDCl₃ | s |
| 98 | (6-Dibromomethyl-2-oxo-2H-chromen-5-yl)-di-carbamic aciddi-tert-butyl ester | 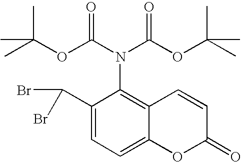 | GC-4-222400 MHzCDCl₃ | s |
| 99 | (6-Formyl-2-oxo-2H-chromen-5-yl)-di-carbamic acid di-tert-butylester | 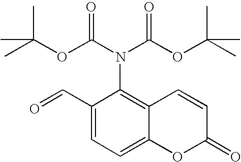 | GC-4-223300 MHzCDCl₃ | NA |
| 100 | (2-Oxo-6-[2-(2-trimethylsilanyl-ethoxy)-vinyl]-2H-chromen-5-yl)-di-carbamic acid di-tert-butylester | 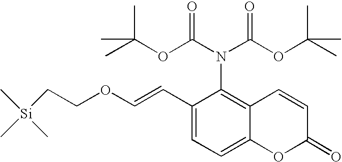 | NA | NA |
| 101 | | 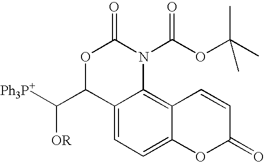 | NA | NA |
| 102 | (6-Bromomethyl-2-oxo-2H-chromen-5-yl)-di-carbamic acid di-tert-butyl ester | 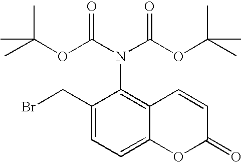 | GC-4-238400 MHzCDCl₃ | s |
| 103 | (6-Cyanomethyl-2-oxo-2H-chromen-5-yl)-di-carbamic acid di-tert-butyl ester | 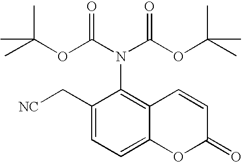 | GC-4-239400 MHzCDCl₃ | s |
| 104 | [2-Oxo-6-(2-oxo-ethyl)-2H-chromen-5-yl]-di-carbamic aciddi-tert-butyl ester | 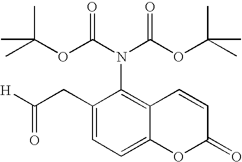 | NA | NA |
| 105 | 6-Bromomethyl-chromen-2-one | 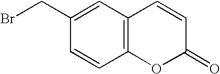 | GC-4-220400 MHzCDCl₃ | s |
| 106 | (2-Oxo-2H-chromen-6-yl)-acetaldehyde | 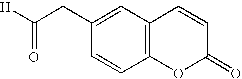 | GC-4-226400 MHzCDCl₃ | s |
| 107 | (2-Oxo-2H-chromen-6-yl)-acetonitrile | 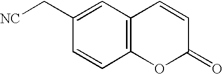 | GC-4-246400 MHzCDCl₃ | s |
| 108 | (2-Oxo-2H-chromen-6-yl)-acetic acid | 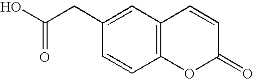 | GC-4-247400 MHzCDCl₃ | s |
| 109 | Acetic acid 2-(5-nitro-2-oxo-2H-chromen-6-yl)-ethyl ester | 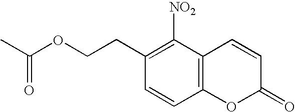 | GC-4-228300 MHzCDCl₃ | s |
| 110 | Acetic acid 2-(5-amino-2-oxo-2H-chromen-6-yl)-ethyl ester | 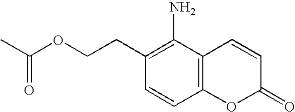 | GC-4-249400 MHzCDCl₃ | s |
| 111 | Acetic acid 1-acetoxy-2-(5-nitro-2-oxo-2H-chromen-6-yl)-ethylester | 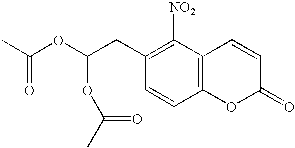 | GC-4-229400 MHzCDCl₃ | s |
| 112 | 8H-Pyrano[3,2-f]indol-2-one | 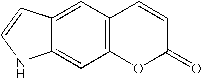 | NA | NA |
| 113 | 1H-9-Oxa-1-aza-cyclopenta[a]naphthalen-8-one | 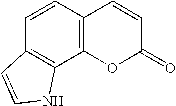 | NA | NA |
| 114 | 3-[4-Amino-3-(2-amino-ethyl)-phenyl]-7-diethylamino-4-methyl-chromen-2-one | 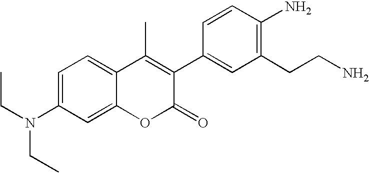 | GC-4-265400 MHzMeOH | s |
| 115 | 6-Amino-5-(2-amino-ethyl)-4-methyl-chromen-2-one | 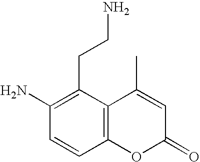 | GC-4-195400 MHzMeOH | NA |
| 116 | 6-Amino-7-(2-amino-ethyl)-chromen-2-one | 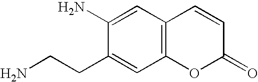 | NG-1-15300 MHzMeOH | s |
| 117 | (2-Amino-5-bromo-phenyl)-aceticacid tert-butyl ester | 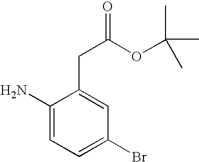 | d400 MHzCDCl₃ | s |
| 118 | (5-Bromo-2-tert-butoxycarbonylamino-phenyl)-acetic acid tert-butyl ester | 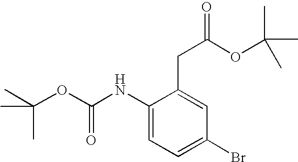 | GC-4-078400 MHzCDCl₃ | s |
| 119 | [4-Bromo-2-(2-hydroxy-ethyl)-phenyl]-carbamic acid tert-butylester | 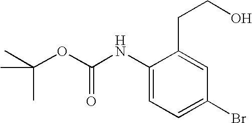 | GC-4-256400 MHzCDCl₃ | s |
| 120 | [2-(2-Azido-ethyl)-4-bromo-phenyl]-carbamic acid tert-butylester | 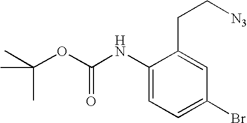 | GC-4-258400 MHzCDCl₃ | s |
| 121 | [4-Bromo-2-(2-tert-butoxycarbonylamino-ethyl)-phenyl]-carbamic acid tert-butylester | 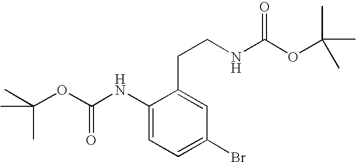 | GC-4-259400 MHzCDCl₃ | s |
| 122 | [2-(2-tert-Butoxycarbonylamino-ethyl)-4-(4,4,5,5-tetramethyl)-[1,3,2]dioxaborolan-2-yl)-phenyl]-carbamicacid tert-butyl ester | 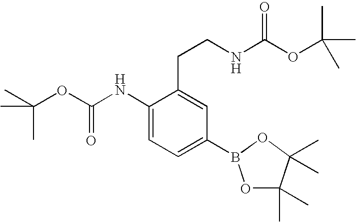 | NA | NA |
| 123 | [2-(2-tert-Butoxycarbonylamino-ethyl)-4-(7-diethylamino-4-methyl-2-oxo-2H-chromen-3-yl)-phenyl]-carbamic acid tert-butyl ester | 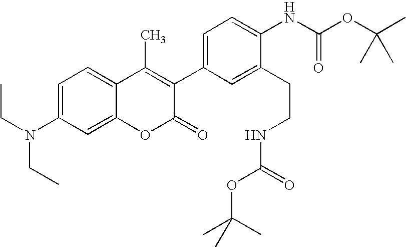 | GC-4-260400 MHzCDCl₃ | s |
| 124 | {5-[2-(10,11-Didhydro-5H-dibenzo[a,d]cyclohepten-5-ylamino)-ethyl]-4-methyl-2-oxo-2H-chromen-6-yl}-di-carbamic acid di-tert-butyl ester | 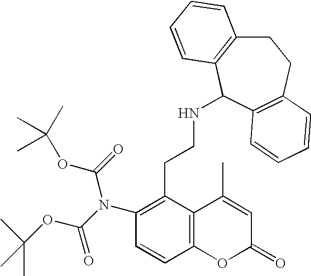 | GC-4-194400 MHzCDCl₃ | s |
| 125 | 7-Amino-3a-methyl-3a,4,5,6-tetrahydro-3H-1-oxa-4-aza-phenalen-2-one | 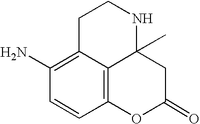 | GC-4-195300 MHzCDCl₃ | s |
| 126 | 7-[2-(10,11-Dihydro-5H-dibenzo[a,d]cyclohepten-5-ylamino)-ethyl]-6-nitro-chromen-2-one | 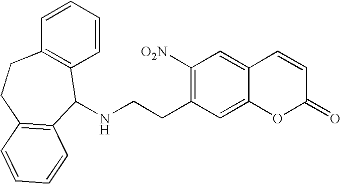 | NG-1-13300 MHzCDCl₃ | s |
| 127 | 6-Amino-7-[2-(10,11-dihydro-5H-dibenzo[a,d]cyclohepten-5-ylamino)-ethyl]-chromen-2-one | 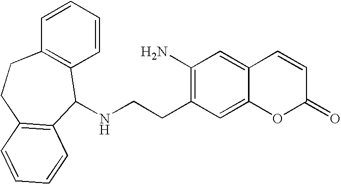 | NG-1-14400 MHzCDCl₃ | s |

Second Series of Experiments

Preparation of Suitable Probes

A novel oxidative deamination coupled with pyrrole-formation cascade reporting mechanism was envisaged to afford a profound electronic change of the system, which led to a change in fluorescence (FIG. 1B). In order to develop a successful probe for MAO, three key criteria need to be fulfilled: (1) the probe should undertake the desired chemical transformation; (2) the chemical transformation should lead to a distinct fluorescence switch; (3) most importantly, the probe could be accepted as a substrate of the enzyme.

As shown in the FIG. 1B, enzymatic oxidation of the ethylamino group is expected to afford an aldehyde intermediate, which would undergo spontaneous intramolecular condensation with the aniline amino group, furnishing an indole moiety in an irreversible fashion. The overall chemical process involves the conversion of an aniline moiety into an indole moiety. It was hypothesized that the chemical transformation was likely to yield a significant emission change of the linked fluorophore through some photo-induced processes like electron transfer or charge transfer (Lakowicz, J. R. Principles of fluorescence spectroscopy (2nd Ed) 1999, Kluwer Academic/Plenum Publishers, NY). Because of limited formulated information about the prediction of fluorescence outcome, an empirical synthetic approach to facilitate design and evaluation was employed.

The core motif of generation I probes was designed based on a hypothesized Photo-induced Electron Transfer (PET) quenching mechanism that the strong electron-donating aniline amino group quenches emission of the linked coumarin fluorophore, while the fluorescence is partially recovered because of the much weaker quenching efficiency of the formed indole (FIG. 2A) (Tanaka, K.; Miura, T.; Umezawa, N.; Urano, Y.; Kikuchi, K.; Higuchi, T.; Nagano, T. J. Am. Chem. Soc. 2001, 123:2530-2536).

Probes 1 and 2 were synthesized and evaluated in terms of the photophysical properties and chemical behavior. As shown in the FIG. 2B, the probes possessed the desired fluorescence switch (12-fold increase in emission intensity) and emission wavelength. Additionally, the aldehyde intermediate underwent a rapid condensation with the aniline amino group to furnish the desired indole moiety. Therefore fluorescence readout of the indole product indeed signaled the true rate-determine oxidative step of MAO action.

Higher switch sensitivity and smaller probe size were pursued. The core motif of generation II probes was designed based on a hypothesized Twisted Intramolecular Charge Transfer (TICT) quenching mechanism that the free rotating 6-amino group of coumarin 3 quenches its fluorescence, while the emission is mostly recovered when the nitrogen is rigidified in the formed pyrrole ring (FIG. 2C) (Rettig, W.; Klock, A. Can. J. Chem. 1985, 63:1649-1653).

Probes 3 and 4 did possess a greatly enhanced fluorescence switch (FIG. 2D). However, the unexpected chemical instability of probe 3, leading to the side product 5 (FIG. 3), precluded it from being a useful reporting probe.

Because of the superior fluorescence switch of aminocoumarin 3 and pyrrolocoumarin 4, attention was turned to the simple pyrrolocoumarins with different structural features.

5,6,7-Aminocoumarins and corresponding pyrrolocoumarins were synthesized and evaluated, see FIG. 3. 5-aminocoumarin 7 and its pyrrolocoumarin 6 possessed excellent fluorescence switch, however, the low nucleophilicity of 5-amino group in the aldehyde intermediate 8 cannot furnish the required rapid pyrrole formation. Flipping the ethylamine side chain of probe 3 to position 7 afforded probe 9. It not only solved the stability problem by preventing the intramolecular cyclization, but an excellent fluorescence switch was reserved in probes 9 and 10. The strong nucleophilicity of 6-amino secured the required rapid pyrrole formation. Compound 11 was eliminated because of its undesired fluorescence switch.

Whether probe 9 would be a suitable substrate for MAOs. In order to approximate more closely the environment in which these enzymes function in vivo, mitochondrial preparations were utilized rather than the semi-purified enzymes. MAO-A in human placental mitochondria(which express MAO-A activity only) and MAO-B in beef liver mitochondria (which express MAO-B activity only) was incubated with the probe; the resulting mixtures were analyzed fluorimetrically following certain time (Bissel, P.; Bigley, M. C.; Castagnoli, K.; Castagnoli, N., Jr. Bioorg. Med. Chem. 2002, 10:3031-3041).

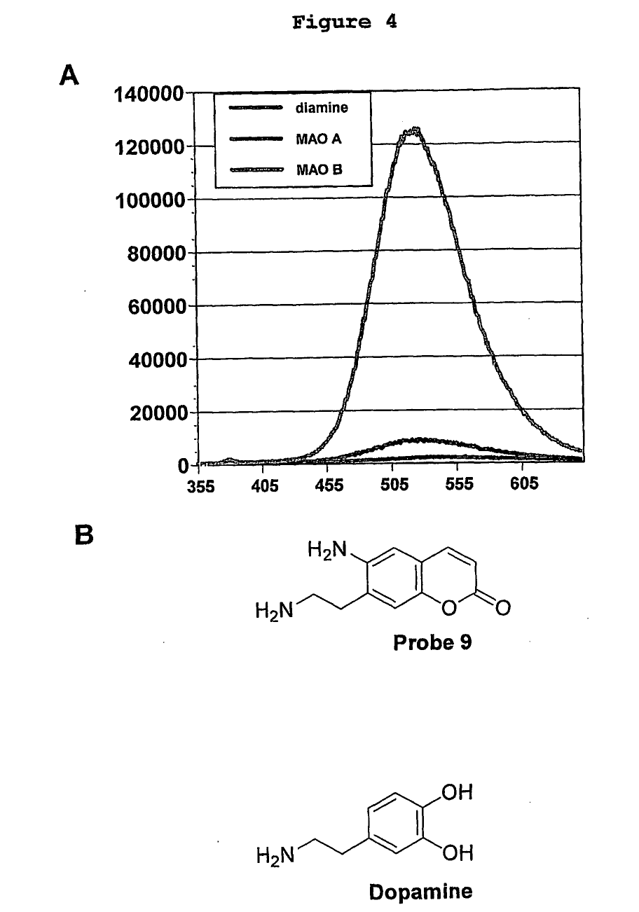

The fluorescence of the mixture increased dramatically after incubation (FIG. 4A). In addition, selectivity of probe 9 as a substrate for MAO-B over MAO-A was observed. Moreover, conversion by the mitochondria was abolished by 1-deprenyl, a known inhibitor of the MAO-B. Subsequent quantitative measurements afforded the kinetic parameters (Km: 13 μM, Kcat: 0.3 min-1) (FIG. 4B). In terms of enzymatic efficiency (kcat/Km), probe 9 is close to its natural substrate dopamine (Rebrin, I.; Geha, R. M.; Chen, K.; Shih, J. C., J. Biol. Chem. 2001, 276:29499-29506).

TABLE 2
Photophysical properties for diamines probes
used in enzymatic screening.^{‡}

| | |
|---|---|
| | I |
| 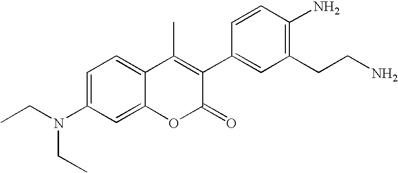 | |
| | III |
| 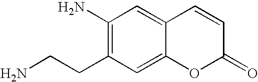 | |
| | IV |
| 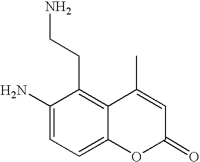 | |
| | V |
| 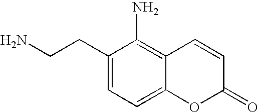 | |

| DIAMINES | III | IV | | V |
|---|---|---|---|---|
| COMPOUND | λₘₐₓ (nm) | ε (M⁻¹cm⁻¹) | λ_{cm} (nm) | Φ |
| I (GC265) | 390 | 13,000 ± 1,000 | 484 | |
| III (NG15)* | 352 | 2,000 ± 100 | — | 0.0017 ± |
| | | | | 0.0001^{a} |
| IV (GC195) | | | | 0.010 ± |
| | | | | 0.003^{a} |
| V (GC264) | 299 | 6,000 ± 100 | — | |

^{‡}all measurements performed in water.
^{a}relative to 9, 10-diphenyl anthracene as a standard (excited at 340 nm);
^{b}relative to 9, 10-diphenyl anthracene as a standard (excited at 365 nm);
^{c}relative to coumarin 6 as a standard (excited at 420 nm)

TABLE 3
Photophysical properties for hydroxyl-amine
probes synthesized for enzymatic screening.^{‡}

| | |
|---|---|
| | VI |
| 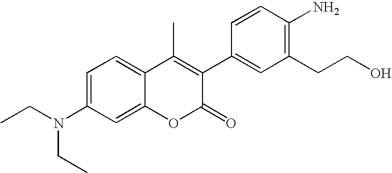 | |
| | VII |
| 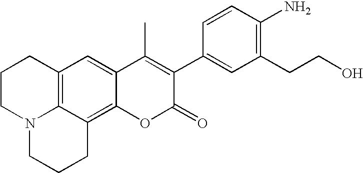 | |
| | VIII |
| 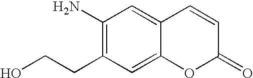 | |
| | IX |
| 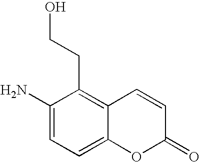 | |
| | X |
| 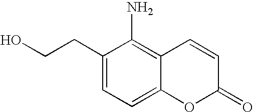 | |

| DIAMINES | VII | VIII | | IX |
|---|---|---|---|---|
| COMPOUND | λₘₐₓ (nm) | ε (M⁻¹cm⁻¹) | λ_{cm} (nm) | Φ |
| VI (GC117) | 394 | 15,000 ± 1,000 | 499 | 0.014 ± |
| | | | | 0.005^{b} |
| VII (GC130) | 391 | 21,000 ± 2,000 | 499 | 0.0040 ± |
| | | | | 0.0006^{b} |
| VIII (NG17)* | 354 | | — | |
| IX (GC161) | 357 | 3,400 ± 500 | — | 0.0017 ± |
| | | | | 0.0006^{b} |
| X (GC251) | 298 | 13,000 ± 3,000 | | |

^{‡}all measurements performed in water.
^{a}relative to 9, 10-diphenyl anthracene as a standard (excited at 340 nm);
^{b}relative to 9, 10-diphenyl anthracene as a standard (excited at 365 nm);
^{c}relative to coumarin 6 as a standard (excited at 420 nm)

TABLE 4
Properties for indoles synthesized for photophysical evaluation.^{‡}

| | |
|---|---|
| | XI |
| 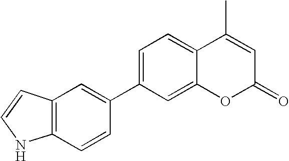 | |
| | XII |
| 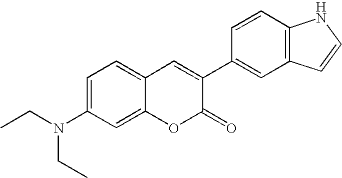 | |
| | XIII |
| 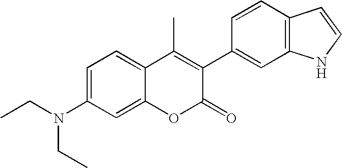 | |
| | XIV |
| 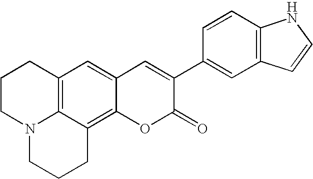 | |
| | XV |
| 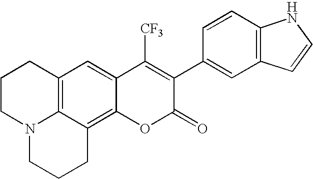 | |
| | VI |
| 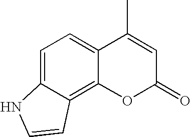 | |
| | XVII |
| 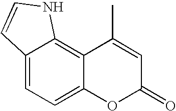 | |
| | XVIII |
| 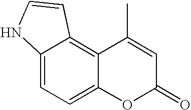 | |
| | XIX |
| 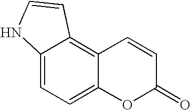 | |
| | XX |
| 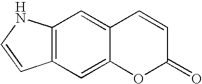 | |

| | INDOLES | | | |
|---|---|---|---|---|
| | λₘₐₓ | | λₘₐₓ | |
| COMPOUND | (nm) | ε (M⁻¹cm⁻¹) | (nm) | Φ |
| XI (GC88) | 339 | ND | 445 | 0.00329 ± 0.00008^{a} |
| XII (GC126) | 401 | 17,000 ± 1,000 | 477 | 0.08 ± 0.02^{c} |
| XIII (GC131) | 399 | 20,000 ± 3,000 | 484 | 0.0103 ± 0.0003^{b} |
| XIV (GC114) | 388 | — | 501 | 0.050 ± 0.005^{b} |
| XV (GC128) | 439 | ND | 557 | ND |
| XVI (GC95) | 325 | ND | 402 | 0.018 ± 0.002^{a} |
| XVII (GC214) | 330 | 14,000 ± 3,000 | 488 | 0.0531 ± 0.0001^{a} |
| XVIII (GC134) | 349 | 11,600 ± 800 | 492 | 0.18 ± 0.05^{a} |
| XIX (GC165) | 355 | 9,400 ± 900 | 517 | 0.05 ± 0.02^{c} |
| XX (NG12) | 335 | 17,000 ± 2,000 | 524 | 0.0082 ± 0.0004^{a} |

^{‡}all measurements performed in water.
^{a}relative to 9, 10-diphenyl anthracene as a standard (excited at 340 nm);
^{b}relative to 9, 10-diphenyl anthracene as a standard (excited at 365 nm);
^{c}relative to coumarin 6 as a standard (excited at 420 nm);
ND = not determined
Further characterization was performed, as shown in table 5.

TABLE 5

| | |
|---|---|
| | 1 |
| 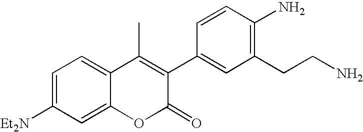 | |
| | 2 |
| 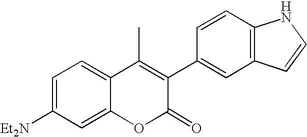 | |
| | 3 |
| 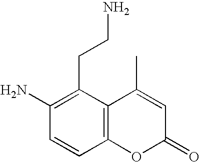 | |
| | 4 |
| 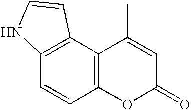 | |
| | 6 |
| 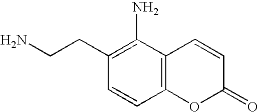 | |
| | 7 |
| 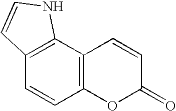 | |
| | 9 |
| 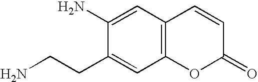 | |
| | 10 |
| 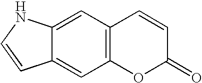 | |
| | 12 |
| 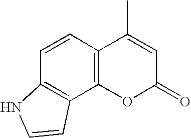 | |
| | 13 |
| 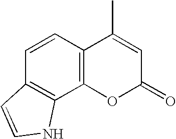 | |

| Compound | λₘₐₓ (nm) | ε (M⁻¹cm⁻¹) | λ_{cm} (nm) | Φ_{EtOH} | Φ_{H2O} |
|---|---|---|---|---|---|
| 1 | 390 | 13,000 ± 1,000 | 484 | 0.0287 ± 0.0006^{b} | 0.00112 ± 0.00008^{b} |
| 2 | 401 | 17,000 ± 1,000 | 477 | 0.59 ± 0.09^{b} | 0.08 ± 0.01^{b} |
| 3 | 350 | ND | 492 | ND | 0.010 ± 0.003^{a} |
| 4 | 349 | 11,600 ± 800 | — | 0.36 ± 0.07^{a} | 0.13 ± 0.02^{a} |
| 6 | 299 | 6,000 ± 100 | 488 | 0.0030 ± 0.0007^{a} | ND |
| 7 | 330 | 14,000 ± 3,000 | — | 0.061 ± 0.0007^{a} | 0.0531 ± 0.0001^{a} |
| 9 | 352 | 2,000 ± 100 | 545 | 0.0105 ± 0.0007^{a} | 0.0017 ± 0.0001^{a} |
| 10 | 335 | 17,000 ± 2,000 | 524 | 0.08 ± 0.01^{a} | 0.0082 ± 0.0004^{a} |
| 12 | 325 | ND | 402 | 0.03 ± 0.01^{a} | 0.018 ± 0.002^{a} |
| 13 | ND | ND | ND | 0.11 ± 0.03^{a} | ND |

^{a}relative to 9, 10-diphenyl anthracene as a standard (excited at 340 nm);
^{b}relative to 9, 10-diphenyl anthracene as a standard (excited at 365 nm)

For Table 5, ultraviolet spectra were measured on a Molecular Devices SPECTRAmax Plus 384 UV-Visible spectrophotometer operated through a Dell Pentium PC by SOFTmax software. All spectra were recorded in 100 mM sodium phosphate (pH 7.4) unless otherwise indicated. Recorded λmax is that of the longest wavelength transition. Extinction coefficients were reported as the average of at least three independent preparations of the probes. Fluorescence measurements were taken on a Jobin Yvon Fluorolog fluorescence spectrofluorometer (slits 3, HV 750) in 100 mM sodium phosphate pH 7.4 buffer unless otherwise indicated. Quantum yields were measured relative to 9,10 diphenylanthracene in EtOH.4 Reported quantum efficiencies are the average of at least three independent preparations of the probes. In the embodiments of this invention the emission and excitation wavelengths can be as set forth in the above tables, including table 5, and can include a range of up to 10 nm or 20 nm above and below the excitation and emission values set forth in the table.

Spectral Comparisons

Apparent fluorescence increase was calculated by comparing the slopes (arbitrary fluorescence units/μM) of standard curves of chemically synthesized product and substrate concentration versus fluorescence intensity. Calculation of the apparent fluorescence increase is shown below for probe 9, which has a 21000% apparent fluorescence increase in emission intensity at λem=524 nm. The constants f_{P} and f_{S} were calculated from the slope of a line of fluorescence intensity versus concentration of chemically synthesized product indole 10 and substrate 9, respectively. These slopes were obtained from calibration curves of at least five concentrations of 9 and 10 in phosphate buffer (pH 7.4). The preparations of the compounds in mitochondrial assay buffer have negligible effects on f_{S} and f_{P}.

$$Fluorescence increase = \frac{{f}_{p} - {f}_{s}}{{f}_{s}} \times 100 \% .$$

Photo-Physical Characterization

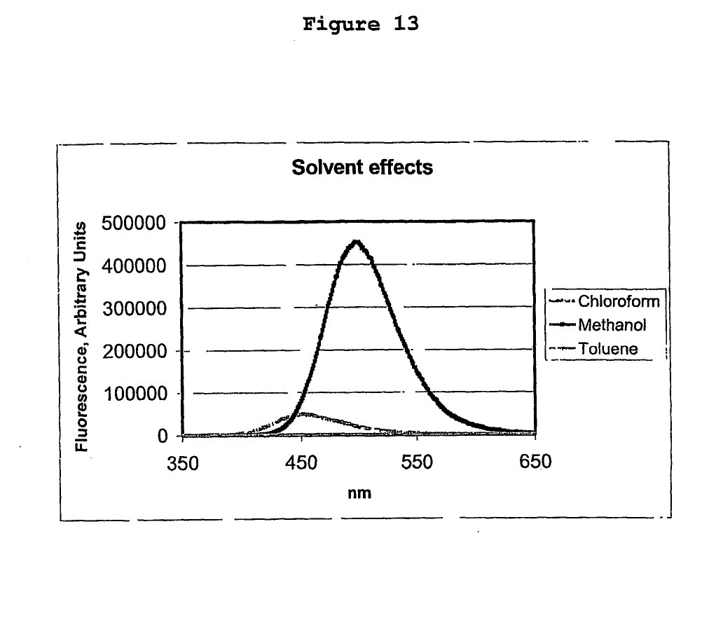

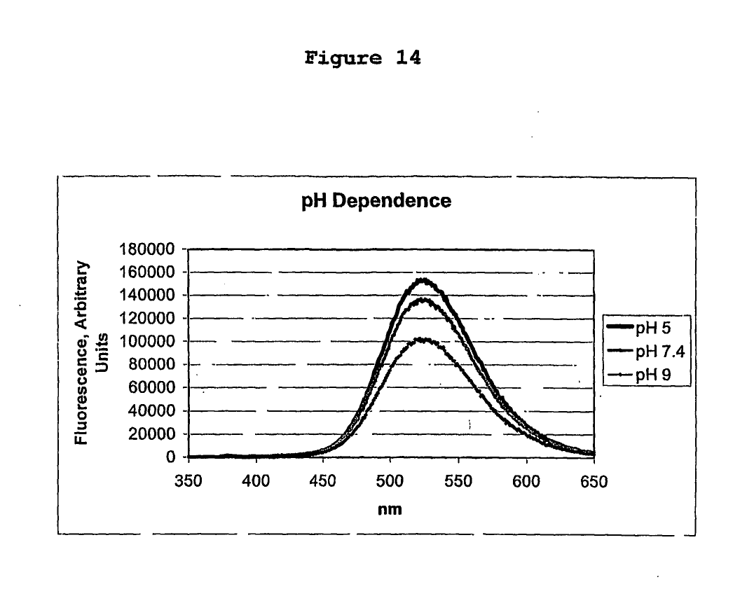

The extinction coefficient ε relates the absorbance of a compound to its concentration as expressed in Beer's law (A=εbc where A is absorbance, b is pathlength, and c is concentration). Extinction coefficients were the average of three independent determinations of three different readings of absorbance from the lowest energy transition at three concentrations. The quantum yield of fluorescence is the fraction of absorbed photons that lead to fluorescence; the number of photons fluoresced divided by the number of photons absorbed. To obtain the quantum yields of the synthesized coumarines their efficiency was compared to standard 9,10-diphenylanthracene at 340 nm in three different determinations. The effect of different solvents on the fluorescence of the compounds was also tested. The compounds were dissolved in methanol, chloroform and toluene and their emissions spectrum was taken. The spectrum of indole (XX) dissolved in different organic solvents is shown in FIG. 13. Then the pH dependence of the compounds was tested; the spectrum of indole (XX) is shown in FIG. 14.

Enzymology

Monoamine Oxidase Assays:

Professor Castagnoli from the Department of Chemistry at the Virginia Polytechnic Institute and State University provided human placenta and beef liver mitochondrial homogenates. Mitochondrial preparations were obtained rather then semi-purified enzyme in these studies in order to approximate more closely the environment in which MAO functions in vivo. Human placental mitochondria, which only expresses MAO A, and beef liver mitochondria, which only expresses MAO B, were prepared using the methodology reported earlier by Salach and were stored at −80° C. prior to use (1. Salach, J. I.; Weyler, W. In Methods in Enzymology; Kaufman, S., Ed.; Academic: London, 1987; Vol. 142, p 627.). The phosphate buffers were prepared using Na₂HPO₄ and NaH₂PO₄. In order to obtain the protein concentrations in the mitochondria by the Bradford assay, the mitochondrial preparations were diluted 1:20 with phosphate buffer containing 50% glycerol. The estimation of MAO concentrations in mitochondria reported earlier by Castagnoli (0.12 nmol MAO-A/mg protein, 0.05 nmol MAO-B/mg protein) was used to obtain the actual concentration of the isozymes in the mitochondria (Castagnoli et al., Bioorg Med Chem. Sep. 10, 2002;(9):3031-41).

Determination of Enzymatic Activity.

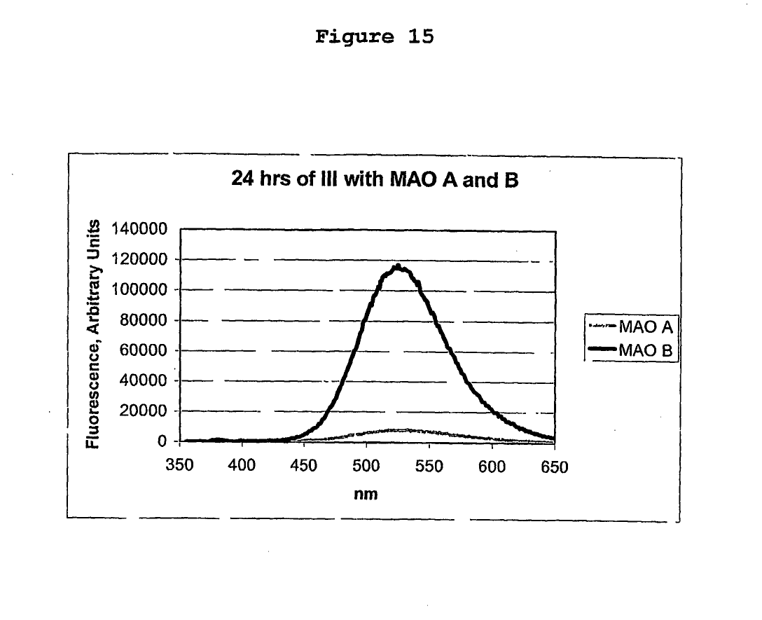

Activity of synthesized diamines with monoamine oxidase A and B were determined by 24-hour incubations at room temperature with 10 microliters of either human placenta mitochondria (MAO-A) or beef liver mitochondria (MAO-B) per milliliter of total assay volume. A twenty-microliter aliquot of the respective diamine (prepared as a 2.5 millimolar stock concentration in dimethyl sulfoxide) were dissolved in 970 microliters of 100 mM sodium phosphate buffer (pH 7.4). Reactions were initiated with 10 microliters of a 30 mg/mL or 70 mg/mL mitochondrial protein mixture (as determined by standard Bradford assay) from human placenta and beef liver, respectively. Enzyme-catalyzed indole formation was realized after 24 hours by reading fluorescence emission upon excitation at the absorbance maxima of the respective indole. Only diamine III showed a significant conversion to its indole XX, the spectras are shown in FIG. 15.

Derivation of Enzyme Kinetic Parameters of Diamine III

Substrate Studies with MAO-B

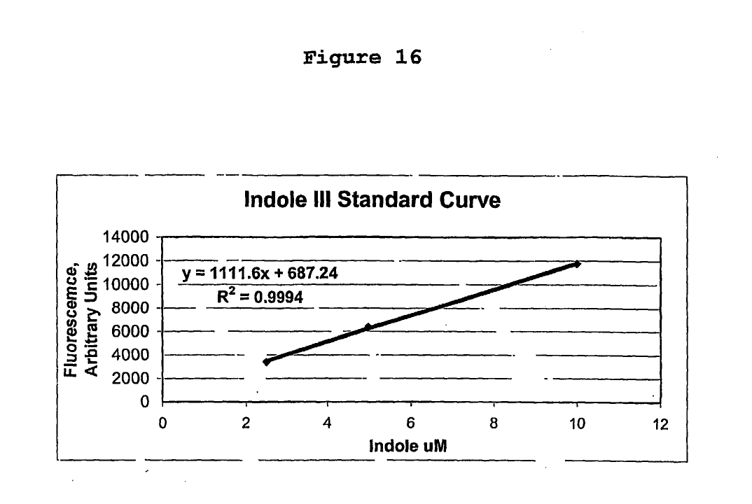

Studies to obtain the Km value for diamine III were performed in triplicates as follows. 64 microliters of a range of stock preparations serially diluted 1 to 1 (1.25 to 0.0048828 mM in 1 m pH 5 phosphate buffer) were added to FALCON black microtiter 96-well plates. 136 microliters of 100 mM sodium phosphate buffer (pH 7.4), prepared with 10 microliters of MAO-B per milliliter of phosphate buffer was then added to the each well to produce final assay volumes of 200 microliters, with total diamine concentrations ranging from 400 micromolar to 1.5625 micromolar and a total estimated MAO-B concentration of 23.8 nM. Product formation from the resulting assay solutions was then assessed via fluorescence detection by a Micromax 386 connected to a Jobin-Yvon Fluorolog through fiber optic cables. Emission at 524 nm resulting from excitation at 335 nm was determined every ten minutes for one hour. Enzymatic rates were calculated from the slope of the fluorescence growth curve and related to a standard curve of the fluorescence of the product III, shown in FIG. 16. The Km and Vmax values, calculated from double reciprocal plots (1/v vs. 1/[S]), revealed that the diamine III is a good substrate for MAO-B (Km=13 μM).

Substrate Studies with MAO-A

Studies to obtain the Km and value for diamine III were performed in duplicates as follows. 32 microliters of a range of stock preparations serially diluted 1 to 1 (0.625 to 0.009765625 mM in 0.1 M pH 7.4 phosphate buffer) were added to FALCON black microtiter 96-well plates. 68 microliters of 100 mM sodium phosphate buffer (pH 7.4), prepared with 50 microliters of dissolved MAO-A per milliliter of phosphate buffer was then added to the each well to produce final assay volumes of 100 microliters, with total diamine concentrations ranging from 200 micromolar to 3.125 micromolar and a total estimated MAO-A concentration of 57.2 nM. Product formation from the resulting assay solutions was then assessed via fluorescence detection by a Micromax 386 connected to a Jobin-Yvon Fluorolog through fiber optic cables. Emission at 524 nm resulting from excitation at 335 nm was determined every minute for one hour. Enzymatic rates were calculated from the slope of the fluorescence growth curve and related to a standard curve of the fluorescence of the product XX. The Km and Vmax values, calculated from double reciprocal plots (1/v vs. 1/[S]), revealed that the diamine III is a good substrate for MAO-A (Km=31 μM). These results indicate that diamine probe III is a good substrate for monoamine oxidase and could be used as a useful fluorescent reporter for monoamine oxidase.

TABLE 6

| | | | | Vₘₐₓ | |
|---|---|---|---|---|---|
| | | k_{cx} | K_{cx}/Kₘ | (nmol min⁻¹ mg⁻¹) | |
| ENZYME | Kₘ (μM) | (min⁻¹) | (min⁻¹ μM⁻¹) | mitochondria | MAO |
| Monoamine | 30.9 ± 1.8 | 0.475 | 0.015 | 0.0570 | 7.96^{a} |
| Oxidase A | | | | | |
| Monoamine | 510 ± 40 | 20.62 | 0.040 | 1.02 | 350^{b} |
| Oxidase B | | | | | |

^{a}Value approximated using the estimated number of 0.12 nmol MAO-A/mg human placental mitochondrial protein⁹;
^{b}Value approximated using the estimated number of 0.05 nmol MAO-A/mg human placental mitochondrial protein.¹⁰

1. A compound having the structure: 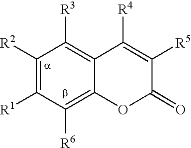
wherein R¹ is —H, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical,
or R¹ and R² form an unsubstituted pyrrole,
or R¹ and R⁶ form an unsubstituted pyrrole,
or R¹ forms an octahydro-quinolizine with R² and R⁶,
wherein each of R², R³, R⁴, R⁵, or R⁶is independently —H, —OH, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O alkyl, —O-alkenyl, —O-alkynyl, —O-aryl, —O-cycloalkyl, —CX₃ where X is a halide, halide, indole radical, or R² and R³ form a pyrrole,
wherein when R¹ and R² form an unsubstituted pyrrole, a nitrogen of the pyrrole is covalently bound to carbon α,
wherein when R¹ and R⁶ form an unsubstituted pyrrole, then R⁴ is —H,
wherein when R¹ is —N(alkyl)₂ or —NH₂, then R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or an indole radical,
wherein when R¹ is H, R³ is —NH₂ and R² is H; or R² and R³ form a pyrrole; or R² is —NH₂ then R⁴ is alkyl,
wherein when R¹ is CH₃, R² is —H, —OH, alkyl, alkenyl, alkynyl, substituted or unsubstituted aryl, cycloalkyl, substituted or unsubstituted heteroaryl, —NH-alkyl, —N(alkyl)₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or an indole radical, and R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or an indole radical,
or a salt or stereoisomer thereof.

2. The compound of claim 1, wherein the indole radical has the structure:

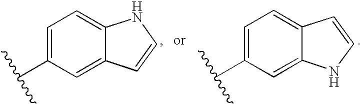

3. The compound of claim 1, wherein the substituted aryl has the structure:

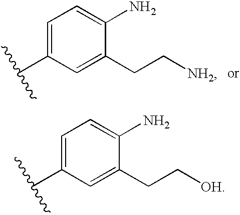

4-20. (canceled)

21. The compound of claim 1, wherein when R² and R³ form a pyrrole, R¹ is alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical or —OH, alkyl, alkenyl, alkynyl, substituted or R⁶ is unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, halide, indole radical.

22. A process of obtaining a compound which is an inhibitor of a monoamine oxidase comprising:
a) providing the monoamine oxidase in a medium;
b) contacting the monoamine oxidase with a known compound that undergoes a detectable increase in fluorescence when oxidized in the presence of the monoamine oxidase under conditions permitting oxidative deamination of the known compound in the presence of the monoamine oxidase;
c) detecting an increase in the fluorescence emitted by the known compound;
d) contacting the monoamine oxidase with a compound to be tested for activity as an inhibitor of monoamine oxidase;
e) detecting a change in the fluorescence of the known compound; and
f) recovering the test compound,
wherein a decrease in the fluorescence emitted by the known compound detected in step e) compared to step c) indicates that the test compound is an inhibitor of monoamine oxidase.

23. The process of claim 22, wherein the monoamine oxidase is MAO-B.

24. The process of claim 22, wherein the monoamine oxidase is MAO-A.

25. The process of claim 22, wherein the known compound has the structure: 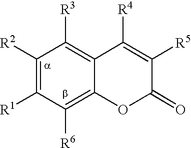
wherein R¹ is —H, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical,
or R¹ and R² form an unsubstituted pyrrole,
or R¹ and R⁶ form an unsubstituted pyrrole,
or R¹ forms an octahydro-quinolizine with R² and R⁶,
wherein each of R², R³, R⁴, R⁵, or R⁶is independently —H, —OH, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, an indole radical, or R² and R³ form a pyrrole,
wherein when R¹ and R² form a pyrrole, a N of the pyrrole is covalently bound to carbon α,
wherein when R¹ and R⁶ form a pyrrole, then R⁴ is —H,
wherein when R¹ is —N(alkyl)₂ or —NH₂, then R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or an indole radical,
wherein when R¹ is H, R³ is —NH₂ and R² is H; or R² and R³ form a pyrrole; or R² is —NH₂ then R⁴ is alkyl,
wherein when R¹ is CH₃, R² is —H, —OH, alkyl, alkenyl, alkynyl, substituted or unsubstituted aryl, cycloalkyl, substituted or unsubstituted heteroaryl, —NH-alkyl, —N(alkyl)₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or an indole radical, and R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or an indole radical, or a salt or stereoisomer thereof.

26-112. (canceled)

113. A method identifying a test compound as an inhibitor of MAO in a solution comprising:
a) providing a solution comprising the MAO;
b) contacting the solution with a compound which undergoes a detectable change in fluorescence when oxidatively deaminated in the presence of a MAO under conditions permitting oxidative deamination of the compound in the presence of the MAO;
c) quantitiating the fluorescence of the compound in the solution and quantitiating any H₂O₂ production in the solution;
d) contacting the sample with the test compound;
e) quantitating the fluorescence of the compound in the solution in the presence of the test compound and quantitiating the H₂O₂ production in the solution in the presence of the test compound;
wherein a decrease in the fluorescence quantitiated in step e) as compared to the fluorescence quantitiated in step c) and a decrease in H₂O₂ production quantitiated in step e) as compared to step c) indicates that the test compound is an inhibitor of the MAO.

114. The method of claim 113, wherein the production of H₂O₂ is quantitiated chemically or by fluorescence.

115. A method identifying a test compound as a substrate of MAO in a solution comprising:
a) providing a solution comprising the MAO;
b) contacting the solution with a compound which undergoes a detectable change in fluorescence when oxidatively deaminated in the presence of a MAO under conditions permitting oxidative deamination of the compound in the presence of the MAO;
c) quantitiating the fluorescence of the compound in the solution and quantitiating any H₂O₂ production in the solution;
d) contacting the sample with the test compound;
e) quantitating the fluorescence of the compound in the solution in the presence of the test compound and quantitiating the H₂O₂ production in the solution in the presence of the test compound;
wherein a decrease in the fluorescence quantitiated in step e) as compared to the fluorescence quantitiated in step c) and no change or an increase in the H₂O₂ production quantitated in step e) as compared to step c) indicates that the test compound is a substrate of the MAO.

116. (canceled)

117. The method of claim 113, wherein the monoamine oxidase is MAO-B.

118. The method of claim 113, wherein the monoamine oxidase is MAO-A.

119-121. (canceled)

122. The method of claim 113, wherein the known compound has the structure: 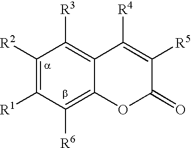
wherein R¹ is —H, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical,
or R¹and R² form an unsubstituted pyrrole,
or R¹ and R⁶ form an unsubstituted pyrrole,
or R¹ forms an octahydro-quinolizine with R and R⁶,
wherein each of R², R³, R⁴, R⁵, or R⁶ is independently —H, —OH, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, halide, indole radical, or R² and R³ form a pyrrole,
wherein when R¹ and R² form a pyrrole, a N of the pyrrole is covalently bound to carbon α,
wherein when R¹ and R⁶ form a pyrrole, then R⁴ is —H,
wherein when R¹ is —N(alkyl)₂ or —NH₂, then R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,
wherein when R¹ is H, R³ is —NH₂ and R² is H; or R² and R³ form a pyrrole; or R² is —NH₂ then R⁴ is alkyl,
wherein when R¹ is CH₃, R² is —H, —OH, alkyl, alkenyl, alkynyl, substituted or unsubstituted aryl, cycloalkyl, substituted or unsubstituted heteroaryl, —NH-alkyl, —N(alkyl)₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical, and R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,
or a salt or stereoisomer thereof.

123-124. (canceled)

125. The compound of claim 1, having the structure:

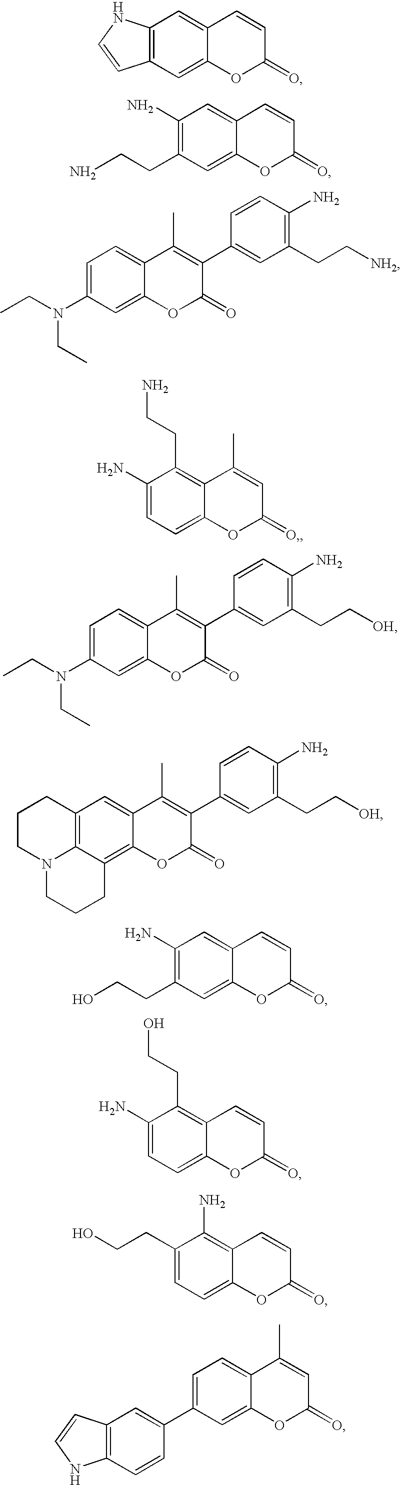

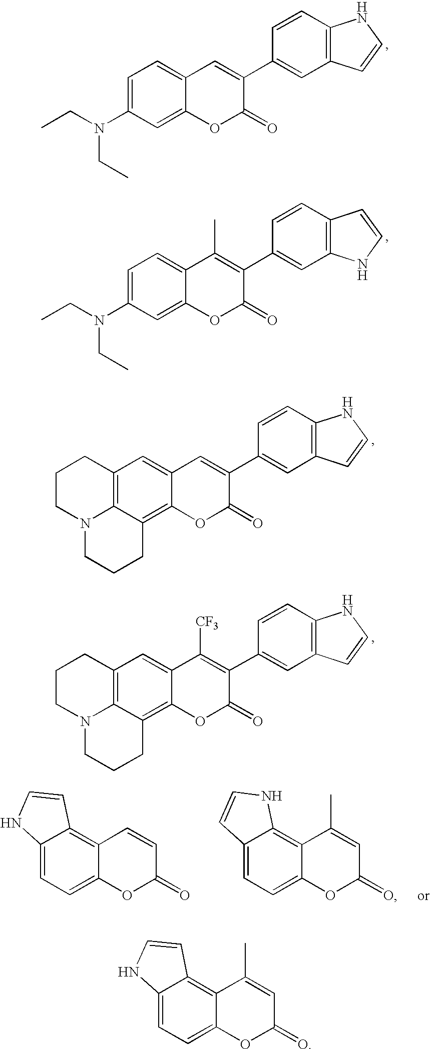

126. A pharmaceutical composition comprising the compound of claim 1 and a pharmaceutically acceptable carrier.

127. A process for producing the compound of claim 1 comprising:
a) contacting a compound having the structure: 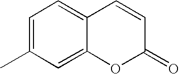
with a suitable nitrating agent in a suitable acid so as to produce a compound having the structure: 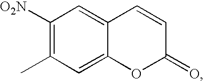
b) condensing the product of step a) in a first suitable solvent in the presence of Me₂NCH(OMe)₂ so as to produce a compound having the structure: 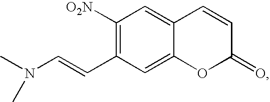
c) hydrolyzing the product of step b) in a refluxing second suitable solvent and aqueous HCL so as to produce a compound having the structure: 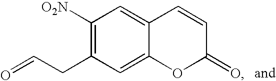
d) hydrogenating the product of step c) with a suitable catalyst in a third suitable solvent,
or
a) contacting a compound having the structure: 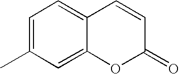
with a suitable nitrating agent in a suitable acid so as to produce a compound having the structure: 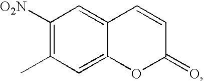
b) condensing the product of step a) in a first suitable solvent in the presence of Me₂NCH(OMe)₂ so as to produce a compound having the structure: 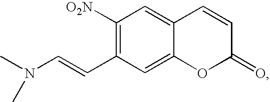
c) hydrolyzing the product of step b) in a refluxing second suitable solvent and aqueous HCL so as to produce a compound having the structure: 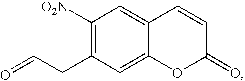
d) reducing the aldehyde of the product of step c) using a suitable reducing agent in a fourth suitable solvent so as to produce a compound having the structure: 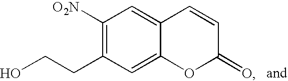
e) hydrogenating the product of step d) with a suitable catalyst in a third suitable solvent,
or
a) reductive amination of a compound having the structure: 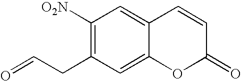
with a suitable reducing agent and a suitable aminating agent in a first suitable solvent so as to produce a compound having the structure: 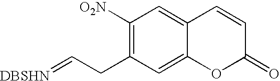
b) reducing the product of step a) by exposing the product of step a) to a second suitable reducing agent in a refluxing second suitable solvent so as to produce a compound having the structure: 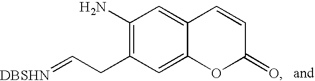
c) removing the amino-protecting DBS group of the product of step b) by exposing the product of step b) to refluxing TFA and quenching with triethylsilane,
or
a) exposing a product having the structure: 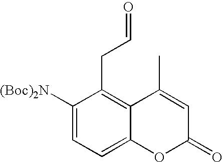
to a suitable reducing agent and a suitable aminating agent in a first suitable solvent so as to produce a compound having the structure: 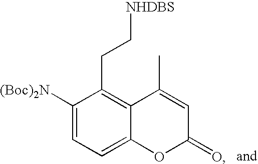
b) removing of the amino-protecting DBS group of the product of step a) by exposing the product of step b) to refluxing TFA and quenching with triethylsilane,
or
a) exposing a compound having the structure: 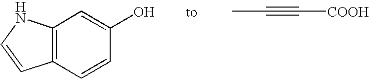
in the presence of DCC and DMAP in a suitable solvent so as to produce a compound having the structure: 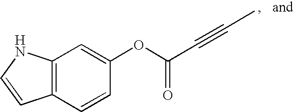
b) exposing the product of step a) to a suitable catalyst and a suitable solvent,
or
a) exposing a compound having the structure: 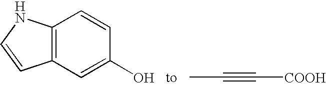
in the presence of DCC and DMAP in a first suitable solvent,
or to ethyl propynoate in the presence of Pd(OAc)₂ in TFA/CH₂Cl₂ or in the presence of Pd₂(dba)₃ in HCOOH, or to a compound having the structure 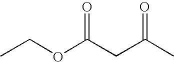
in the presence of concentrated H₂SO₄,
so as to produce a compound having the structure: 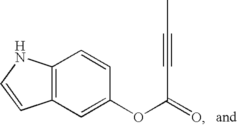
b) exposing the product of step a) to a suitable catalyst and a second suitable solvent,
or
a) exposing a compound having the structure: 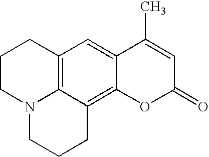
to a brominating agent in a first suitable solvent so as to produce a compound having the structure: 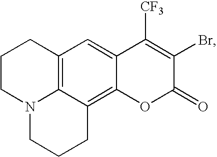
b) coupling the product the product of step a) to a compound having the structure: 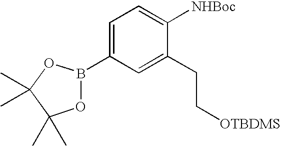
in the presence of a suitable catalyst and a suitable base in dioxane so as to produce a compound having the structure: 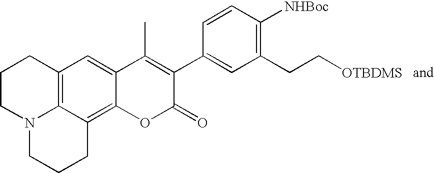
c) deprotecting the product of step b) with TBAF in THF and then TFA in CH₂Cl₂,
or
a) exposing a compound having the structure: 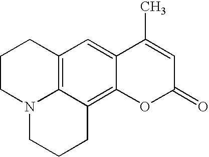
to a brominating agent in a first suitable solvent so as to produce a compound having the structure: 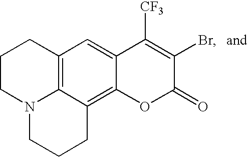
b) coupling the product the product of step a) to a compound having the structure: 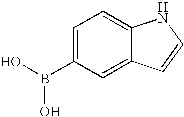
in the presence of a suitable catalyst and a suitable base in a second suitable solvent,
or
a) brominating a compound having the structure: 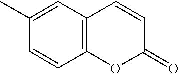
with a suitable brominating agent in the presence of benzoylperoxide in a first suitable solvent so as to produce a compound having the structure: 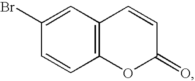
b) exposing the product of step a) to KCN in a second suitable solvent so as to produce a compound having the structure: 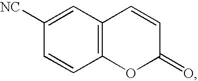
c) exposing the product of step b) to AcOH and H₂SO₄ in water so as to produce a compound having the structure: 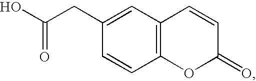
d) reducing the product of step c) with a suitable reducing agent in a third suitable solvent so as to produce a compound having the structure: 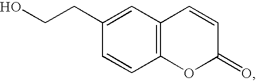
e) nitrating the product of step c) with a suitable nitrating agent and a suitable acid in a fourth suitable solvent so as to produce a compound having the structure: 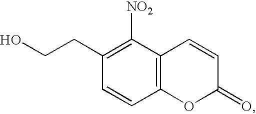
f) catalytically hydrogenating the nitro group of the product of step e) with H₂ and a suitable catalyst in a fifth suitable solvent so as to produce a compound having the structure: 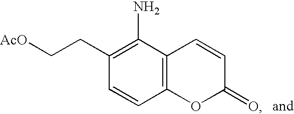
saponifying the product of step f) with a suitable base in a sixth suitable solvent,
or
a) exposing a compound having the structure: 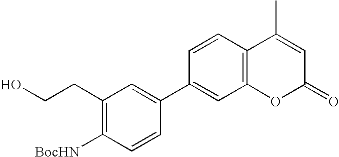
to Dess-Martin periodinane oxidation in the presence of CH₂Cl₂ so as to produce a compound having the structure: 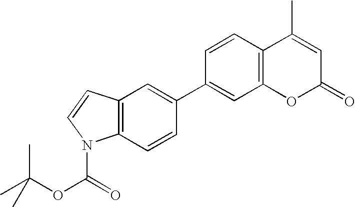
and a compound having the structure: 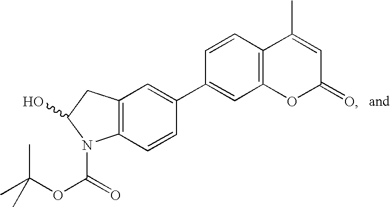
b) exposing either or both of the products of step a) to TFA in CH₂Cl₂,
or
a) exposing a compound having the structure: 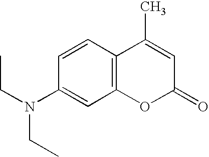
to a suitable brominating agent in the presence of a first suitable solvent so as to produce a compound having the structure: 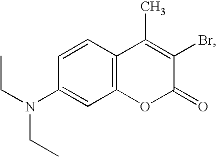
b) coupling the product of step a) with a compound having the structure: 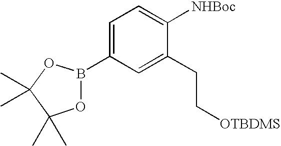
in the presence of a suitable catalyst and a suitable base in dioxane so as to produce a compound having the structure: 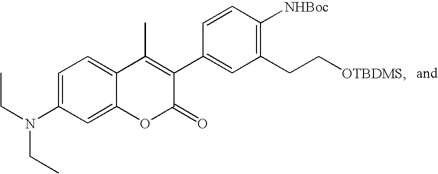
deprotecting the product of step b) with TBAF in THF and then TFA in CH₂Cl₂,
or
a) exposing a compound having the structure: 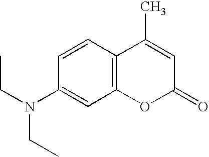
to a suitable brominating agent in the presence of a first suitable solvent so as to produce a compound having the structure: 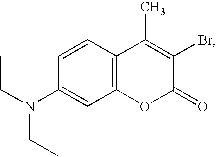
b) coupling the product of step a) with a compound having the structure: 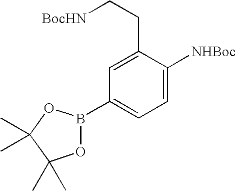
in the presence of a suitable catalyst and a suitable base in dioxane so as to produce a compound having the structure: 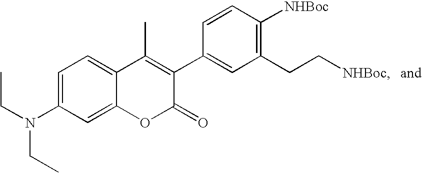
c) deprotecting the product of step b) with TFA in CH₂Cl₂,
or
a) exposing a compound having the structure: 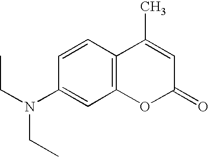
to a suitable brominating agent in the presence of a first suitable solvent so as to produce a compound having the structure: 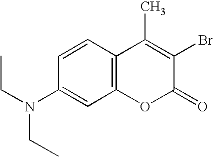
b) coupling the product of step a) with a compound having the structure: 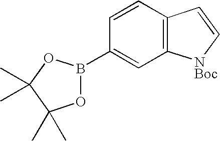
in the presence of a suitable catalyst and a suitable base in dioxane so as to produce a compound having the structure: 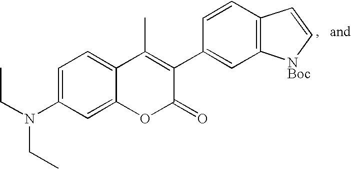
b) deprotecting the product of step b) by heating at a suitable temperature,
or
a) contacting a compound having the structure: 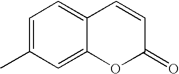
with a suitable nitrating agent in a suitable acid so as to produce a compound having the structure: 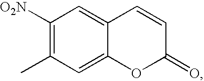
b) condensing the product of step a) in a first suitable solvent in the presence of Me₂NCH(OMe)₂ so as to produce a compound having the structure: 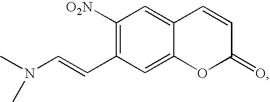
c) hydrolyzing the product of step b) in a refluxing second suitable solvent and aqueous HCL so as to produce a compound having the structure: 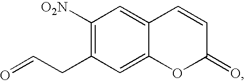
d) aminating the product of step c) with a suitable aminating agent in the presence of a suitable reducing agent and a third suitable solvent so as to produce a compound having the structure: 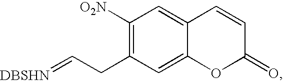
e) reducing the product of step a) by exposing the product of step a) to a second suitable reducing agent in a refluxing second suitable solvent so as to produce a compound having the structure: 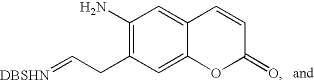
f) exposing the product of step e) to refluxing TFA and quenching with TES,
so as to produce the compound of claim 1.

128. The method of claim 115, wherein the monoamine oxidase is MAO-B.

129. The method of claim 115, wherein the monoamine oxidase is MAO-A.

130. The method of claim 115, wherein the known compound has the structure: 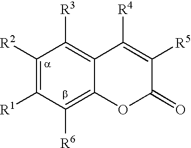
wherein R¹ is —H, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical,
or R¹ and R² form an unsubstituted pyrrole,
or R¹ and R⁶ form an unsubstituted pyrrole,
or R¹ forms an octahydro-quinolizine with R² and R⁶,
wherein each of R², R³, R⁴, R⁵, or R⁶is independently —H, —OH, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, halide, indole radical, or R² and R³ form a pyrrole,
wherein when R¹ and R² form a pyrrole, a N of the pyrrole is covalently bound to carbon α,
wherein when R¹ and R⁶ form a pyrrole, then R⁴ is —H,
wherein when R¹ is —N(alkyl)₂ or —NH₂, then R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,
wherein when R¹ is H, R³ is —NH₂ and R² is H; or R² and R³ form a pyrrole; or R is —NH₂ then R⁴ is alkyl,
wherein when R¹ is CH₃, R² is —H, —OH, alkyl, alkenyl, alkynyl, substituted or unsubstituted aryl, cycloalkyl, substituted or unsubstituted heteroaryl, —NH-alkyl, —N(alkyl)₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical, and R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,
or a salt or stereoisomer thereof.

131. A method of treating a depressive disorder, an anxiety disorder, or a neurodegenerative disease in a subject comprising administering to the subject an effective amount of a compound having the structure: 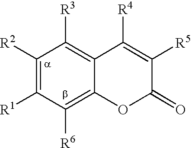
wherein R¹ is —H, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical,
or R¹ and R² form an unsubstituted pyrrole,
or R¹ and R⁶ form an unsubstituted pyrrole,
or R¹ forms an octahydro-quinolizine with R² and R⁶,
wherein each of R², R³, R⁴, R⁵, or R⁶is independently —H, —OH, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, halide, or an indole radical, or R² and R³ form a pyrrole,
wherein when R¹ and R² form a pyrrole, a N of the pyrrole is covalently bound to carbon α,
wherein when R¹ and R⁶ form a pyrrole, then R⁴ is —H,
wherein when R¹ is —N(alkyl)₂ or —NH₂, then R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,
wherein when R¹ is H, R³ is —NH₂ and R² is H; or R² and R³ form a pyrrole; or R² is —NH₂ then R⁴ is alkyl,
wherein when R¹ is CH₃, R² is —H, —OH, alkyl, alkenyl, alkynyl, substituted or unsubstituted aryl, cycloalkyl, substituted or unsubstituted heteroaryl, —NH-alkyl, —N(alkyl)₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical, and R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,
or a salt or stereoisomer thereof.

132. A method for detecting an active monoamine oxidase in a nervous system tissue comprising:
a) providing a sample of the nervous system tissue;
b) contacting the sample with a known compound which undergoes a detectable increase in fluorescence when oxidized in the presence of a monoamine oxidase under conditions permitting oxidative deamination of the compound in the presence of the monoamine oxidase;
c) detecting an increase in the fluorescence of the known compound;
wherein an increase in the fluorescence of the sample detected in step c) indicates the presence of an active monoamine oxidase in the nervous system tissue.

133. A composition comprising a pharmaceutically acceptable carrier and a compound having the structure: 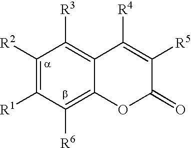
wherein R¹ is —H, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical,
or R¹ and R² form an unsubstituted pyrrole,
or R¹ and R⁶ form an unsubstituted pyrrole,
or R¹ forms an octahydro-quinolizine with R² and R⁶,
wherein each of R², R³, R⁴, R^{5,} or R⁶ is independently —H, —OH, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, halide, indole radical, or R² and R³ form a pyrrole,
wherein when R¹ and R² form a pyrrole, a N of the pyrrole is covalently bound to carbon α,
wherein when R¹ and R⁶ form a pyrrole, then R⁴ is —H,
wherein when R¹ is —N(alkyl)₂ or —NH₂, then R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,
wherein when R¹ is H, R³ is —NH₂ and R² is H; or R² and R³ form a pyrrole; or R² is —NH₂ then R⁴ is alkyl,
wherein when R¹ is CH₃, R² is —H, —OH, alkyl, alkenyl, alkynyl, substituted or unsubstituted aryl, cycloalkyl, substituted or unsubstituted heteroaryl, —NH-alkyl, —N(alkyl)₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical, and R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,
or a salt or stereoisomer thereof.

134. A process for making a composition comprising admixing a carrier and an amount of a compound having the structure: 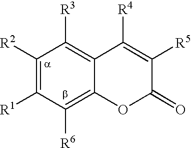
wherein R¹ is —H, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), halide, CX₃ where X is a halide, or indole radical,
or R¹ and R² form an unsubstituted pyrrole,
or R¹ and R⁶ form an unsubstituted pyrrole,
or R¹ forms an octahydro-quinolizine with R² and R⁶,
wherein each of R², R³, R⁴, R⁵, or R⁶is independently —H, —OH, alkyl, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, —CX₃ where X is a halide, halide, indole radical, or R² and R³ form a pyrrole,
wherein when R¹ and R² form a pyrrole, a N of the pyrrole is covalently bound to carbon α,
wherein when R¹ and R⁶ form a pyrrole, then R⁴ is —H,
wherein when R¹ is —N(alkyl)₂ or —NH₂, then R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,
wherein when R¹ is H, R³ is —NH₂ and R² is H; or R² and R³ form a pyrrole; or R² is —NH₂ then R⁴ is alkyl,
wherein when R¹ is CH₃, R² is —H, —OH, alkyl, alkenyl, alkynyl, substituted or unsubstituted aryl, cycloalkyl, substituted or unsubstituted heteroaryl, —NH-alkyl, —N(alkyl)₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical, and R⁴ is —H, —OH, alkenyl, alkynyl, substituted or unsubstituted aryl, cycloalkyl, substituted or unsubstituted heteroaryl, —NH-alkyl, —N(alkyl)₂, —NH₂, -alkyl-C(O)(OH), -alkyl-OH, -alkyl-(NH₂), —O-alkyl, —O-alkenyl, —O-alkynyl, —O-aryl, —O-cycloalkyl, halide, CX₃ where X is a halide, or indole radical,
or a salt or stereoisomer thereof.

135. A method identifying an active MAO in a sample comprising:
a) providing a sample derived from a mammal;
b) contacting the sample with a compound which undergoes a detectable change in fluorescence when oxidatively deaminated in the presence of a MAO under conditions permitting oxidative deamination of the compound in the presence of the MAO;
c) detecting a change in fluorescence of the compound;
wherein an increase in the fluorescence in step c) indicates an active mammalian MAO in the sample.
